(19) United States
(12) Patent Application Publication
(10) Pub. No.: US 2024/0016923 A1
(43) Pub. Date: Jan. 18, 2024

(54) MODIFIED SECRETED HEPATITIS C VIRUS (HCV) E1E2 GLYCOPROTEINS AND METHODS OF USE THEREOF

(71) Applicants: UNIVERSITY OF MARYLAND, COLLEGE PARK, College Park, MD (US); THE BOARD OF TRUSTEES OF THE LELAND STANFORD JUNIOR UNIVERSITY, Stanford, CA (US)

(72) Inventors: Brian G. Pierce, Gaithersburg, MD (US); Thomas R. Fuerst, Gaithersburg, MD (US); Eric A. Toth, Spring Grove, PA (US); Johnathan D. Guest, St. Louis, MO (US); Steven K.H. Foung, Stanford, CA (US); Zhen-Yong Keck, Redwood City, CA (US)

(21) Appl. No.: 18/252,800

(22) PCT Filed: Nov. 12, 2021
(86) PCT No.: PCT/US2021/059171

§ 371 Date: May 12, 2023

Related U.S. Application Data
(60) Provisional application No. 63/113,180, filed on Nov. 12, 2020.
(60) Provisional application No. 63/260,475, filed on Aug. 20, 2021.

(51) Int. Cl. A61K 39/29; A61P 31/14; C07K 14/18

(52) U.S. Cl. CPC A61K 39/29; A61P 31/14; C07K 14/1833

(57) ABSTRACT

Disclosed are modified hepatitis C virus (HCV) E1E2 glycoproteins. Disclosed are Disclosed are modified HCV E1E2 glycoproteins comprising a HCV E1 polypeptide; a first scaffold element; a HCV E2 polypeptide; and a second scaffold element, wherein the HCV E1 polypeptide does not comprise a transmembrane domain, and wherein the HCV E2 polypeptide does not comprise a transmembrane domain. Disclosed are modified HCV E1E2 glycoproteins comprising a HCV E1 polypeptide; a first scaffold element; a modified HCV E2 polypeptide; and a second scaffold element, wherein the HCV E1 polypeptide does not comprise a transmembrane domain; a first scaffold element, wherein the modified HCV E2 polypeptide does not comprise a transmembrane domain, wherein the modified HCV E2 polypeptide comprises an antigenic domain D, and wherein the modified HCV E2 polypeptide comprises one or more amino acid alterations in the antigenic domain D and/or wherein the modified HCV E2 polypeptide comprises an antigenic domain A, wherein the antigenic domain A comprises an N-glycan sequon substitution. Also disclosed are methods of using the disclosed modified HCV E1E2 glycoproteins, such as methods of inducing an immune response in a subject, methods of treating a subject, and methods of increasing antigenicity of a HCV E1E2 glycoprotein.

### CROSS-REFERENCE TO RELATED APPLICATIONS

This application claims the benefit of U.S. Provisional Patent Application No. 63/113,180, filed on Nov. 12, 2020, and U.S. Provisional Patent Application No. 63/260,475, filed on Aug. 20, 2021, each of which is incorporated by reference herein in its entirety.

### STATEMENT REGARDING FEDERALLY SPONSORED RESEARCH

This invention was made with government support under Grant Numbers R01AI132213 and R21AI154100 awarded by the National Institutes of Health. The government has certain rights in this invention.

### BACKGROUND

Hepatitis C virus (HCV) infection is a major global disease burden, with 71 million individuals, or approximately 1% of the global population, chronically infected worldwide, and 1.75 million new infections per year. Chronic HCV infection can lead to cirrhosis and hepatocellular carcinoma, the leading cause of liver cancer, and in the United States HCV was found to surpass HIV and 59 other infectious conditions as a cause of death. While the development of direct-acting antivirals has improved treatment options considerably, several factors impede the effective use of antiviral treatment such as the high cost of antivirals, viral resistance, and occurrence of reinfections after treatment cessation, and lack of awareness of infection in many individuals since HCV infection is considered a silent epidemic.

Despite decades of research resulting in several HCV vaccine candidates tested in vivo and in clinical trials, no approved HCV vaccine is available. There are a number of barriers to the development of an effective HCV vaccine, including the high mutation rate of the virus which leads to viral quasi-species in individuals and permits active evasion of T cell and B cell responses. Escape from the antibody response by HCV includes mutations in the envelope glycoproteins, as observed in vivo in humanized mice, studies in chimpanzee models, and through analysis of viral isolates from human chronic infection. This was also clearly demonstrated during clinical trials of a monoclonal antibody, HCV1, which in spite of its targeting a conserved epitope on the viral envelope, failed to eliminate the virus, as viral variants with epitope mutations emerged under immune pressure and dominated the rebounding viral populations in all treated individuals.

An additional bottleneck contributing to the difficulty in generating protective B cell immune responses required for an effective HCV vaccine is preparation of a homogeneous E1E2 antigen. HCV envelope glycoproteins E1 and E2 form a heterodimer on the surface of the virion. Furthermore, E1E2 assembly has been proposed to form a trimer of heterodimers mediated by hydrophobic C-terminal transmembrane domains (TMDs) and interactions between E1 and E2 ectodomains. These glycoproteins are necessary for viral entry and infection, as E2 attaches to the CD81 and scavenger receptor type B class I (SR-B1) co-receptors as part of a multi-step entry process on the surface of hepatocytes. Neutralizing antibody responses to HCV infection target epitopes in E1, E2, or the E1E2 heterodimer. A significant impediment to the uniform production of an immunogenic E1E2 heterodimer that could be utilized for vaccine development is the association of the antigen with the membrane via the TMDs. Progress has been made in the production and purification of the membrane-bound E1E2 complex via immunoaffinity purification or the use of tags that allow protein A or anti-Flag chromatography. While these methods produce high quality samples, they all involve harsh elution conditions. How such conditions might influence sample quality at a scale required for vaccine trials is unclear. Further, intracellular expression and membrane extraction limits the ability to produce large quantities of sufficient homogeneity required for both basic research and vaccine production. In contrast, viral glycoproteins of influenza hemagglutinin, respiratory syncytial virus (RSV), SARS-CoV-2, and others have been stabilized in soluble form using a C-terminal attached foldon trimerization domain to facilitate assembly. In addition, HIV gp120-gp41 proteins have been designed as soluble SOSIP trimers in part by introducing a furin cleavage site to facilitate native-like assembly when cleaved by the enzyme. Recent efforts have made strides toward liberating the E1E2 complex from the membrane in its native form.

### BRIEF SUMMARY

Disclosed are modified membrane bound hepatitis C virus (HCV) E1E2 glycoproteins.

Disclosed are modified HCV E1E2 glycoproteins comprising a HCV E1 polypeptide; a first scaffold element; a HCV E2 polypeptide; and a second scaffold element, wherein the HCV E1 polypeptide does not comprise a transmembrane domain, and wherein the HCV E2 polypeptide does not comprise a transmembrane domain.

Disclosed are modified HCV E1E2 glycoproteins comprising a HCV E1 polypeptide; a first scaffold element; a modified HCV E2 polypeptide; and a second scaffold element, wherein the HCV E1 polypeptide does not comprise a transmembrane domain, wherein the modified HCV E2 polypeptide does not comprise a transmembrane domain, wherein the modified HCV E2 polypeptide comprises an antigenic domain D, and wherein the modified HCV E2 polypeptide comprises one or more amino acid alterations in the antigenic domain D and/or wherein the modified HCV E2 polypeptide comprises an antigenic domain A, wherein the antigenic domain A comprises an N-glycan sequon substitution.

Disclosed are polynucleotides comprising a nucleic acid sequence capable of encoding one or more of the disclosed modified HCV E1E2 glycoproteins.

Disclosed are vectors comprising any of the polynucleotides disclosed herein.

Disclosed are compositions comprising one or more of the disclosed modified HCV E1E2 glycoproteins described herein and a pharmaceutically acceptable carrier thereof.

Also disclosed are cells or cell lines comprising the compositions, vectors, polynucleotides or modified HCV E1E2 glycoproteins disclosed herein.

Disclosed are methods of increasing HCV E1E2 glycoprotein immunogenicity in a subject in need thereof comprising administering a composition comprising one or more of the disclosed modified HCV E1E2 glycoproteins.

Disclosed are methods of increasing HCV E1E2 glycoprotein antigenicity in a subject in need thereof comprising administering a composition comprising one or more of the modified HCV E1E2 glycoproteins described herein.

Disclosed are method of decreasing HCV E1E2 glycoprotein antigenicity in a subject in need thereof comprising administering a composition comprising one or more of the modified HCV E1E2 glycoproteins having an alteration in the HCV E2 polypeptide antigenic domain A described herein.

Disclosed are methods of inducing an immune response in a subject in need thereof comprising administering to the subject in need thereof a composition comprising one or more of the modified HCV E1E2 glycoproteins disclosed herein.

Disclosed are methods of treating a subject having HCV or at risk of being infected with HCV comprising administering to the subject a composition comprising one or more of the modified HCV E1E2 glycoproteins disclosed herein.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive example aspects of the present disclosure as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1C show a construction and characterization of mbE1E2, sE1E2.LZ, and sE2. FIG. 1A: Schematic diagram of full-length mbE1E2, sE1E2 with c-Fos/c-Jun and furin cleavage sites, and sE2. Signal sequences (tPA and IgK) and 6×His tags are shown.

FIG. 1B: SDS-PAGE analysis of purified mbE1E2, sE1E2.LZ and sE2 under reducing conditions. FIG. 1C: Western blot detection of the purified proteins under reducing and non-reducing conditions using anti-E2 mAb (HCV1) and anti-E1 mAb (H-111) as probes. M: Monomer, D: Dimer, T: Trimer

FIGS. 2A-2E show immunogenicity assessment of antibodies inducted in immunized mice at Day 56 by ELISA. FIG. 2A: anti-mbE1E2 titer. FIG. 2B: anti-sE1E2.LZ titer. FIG. 2C: anti-sE2 titer. FIG. 2D: Binding to E1 peptides, E2 peptides and c-Fos/c-Jun. The numbers presented in panel D represent the average of duplicate experiments. FIG. 2E: Anti-mbE1E2 titers at different days post-immunization. Endpoint titers were calculated by curve fitting in GraphPad Prism software with endpoint OD defined as four times the highest absorbance value of Day 0 sera. In FIG. 2D, peptide binding endpoint titers were calculated by curve fitting in GraphPad Prism software with endpoint OD defined as seven times the highest absorbance value of Day 0. P-values were calculated using Kruskal-Wallis analysis of variance with Dunn's multiple comparison test, and significant p-values are shown (*: p<0.05). Panel A represents previously published data (Guest et al., Proc Natl Acad Sci USA 118 (2021)), shown here for comparison.

FIG. 3 shows competition ELISA using day 56 pooled serum with paired domain specific antibodies of E2 at domain B (AR3A & HEPC74), domain D (HC84.26 & HC84.1), domain E (HCV1 & HC33.1) and anti-E1E2 antibodies (AR4A & AR5A). Serum competition with HCV E1-specific antibodies (HC111 & IGH526) and non-neutralizing antibodies (CBH-4B and CBH-4G) was also analyzed. The value shown is the percentage inhibition relative to pre-immune sera.

FIGS. 4A-4F show competition ELISA of individual mouse Day 56 serum at 1:60 dilutions with anti-E2 antibodies: domain B AR3A (FIG. 4A), domain D HC84.26 (FIG. 4B), domain E HCV1 (FIG. 4C); anti-EE2 antibodies: AR4A (FIG. 4D), AR5A (FIG. 4E); and anti-E1 antibody H111 (FIG. 4F). Data were calculated using Kruskal-Wallis analysis of variance with Dunn's multiple comparison test.

FIGS. 5A-5I show breadth of neutralization against all HCV genotypes with HCVpp. Individual immunized mice sera were assessed for neutralization activities at Day 56 (FIGS. 5A to I) and day 0 (data not shown) against seven genotypes of HCVpp. Neutralization titers were calculated as serum dilution levels reached at 50% neutralization (ID50) by curve fitting in Graphpad Prism software. Serum dilutions were performed as two-fold dilutions starting at 1:64 for HCVpp neutralization. ID50 values are plotted on a log 10 scale on the y-axis. P-values were calculated using Kruskal-Wallis analysis of variance with Dunn's multiple comparison test, and significant p-values are shown (*: p<0.05). The H77C neutralization data (panel A) were previously published (66) and are shown here for comparison.

FIGS. 6A and 6B show kinetics of neutralization. (FIG. 6A) Neutralization titers (ID50s) against homologous isolate (H77C, GT1a). (FIG. 6B) The same shown against heterologous isolate (J6, GT2a) shown in FIG. 5C. Serial dilutions of day 56 (terminal bleed) pooled serum were used and titers were calculated as serum dilution levels reached at 50% neutralization (ID50) by curve fitting in GraphPad Prism software.

FIG. 7 shows breadth of neutralization against all HCV genotypes with HCVcc. Day 56 immunized mice pooled sera were analyzed for neutralization using chimeric HCVcc with H77C (GT1a), J4 (GT1b), Con1/Jc1 (GT1b/2a), J8 (GT2b), S52 (GT3a), ED43 (GT4), SA13 (GT5a), HK (GT6a), QC69 (GT7a). % neutralization was calculated using relative luminescence units (RLU) normalized to RLU of supernatant cultured without HCVcc nor serum (100%) and RLU of supernatant cultured with HCVcc without serum (0%). 50% neutralization was calculated from the sigmoid curve. Dotted line indicates highest concentration of serum.

FIG. 8 shows a heat map ID50 showing heterologous neutralization for three immunized groups. HCVpp neutralization (left panel) and HCVcc neutralization (right panel). Each row corresponds to an HCV genotype represented as HCVpp or HCVcc, and cell colors represent mean group ID50 values from FIG. 5, or pooled serum ID50 values from FIG. 7.

FIGS. 9A-9D show design of sE1E2 constructs. (FIG. 9A) Schematic of mbE1E2, covalent linker sE1E2 constructs, and cleavable polyprotein constructs. Regions shown include wild-type signal peptide (SP), tPA signal sequence, E1 ectodomain, E2 ectodomain, wild-type TMDs, Gly-Ser linker, and various scaffolds replacing TMDs. E1E2 residue ranges for each region are noted according to H77 numbering. C-terminal His tags and furin cleavage sites are shown in boxes and labeled. The expected molecular weight of each construct is indicated, and molecular weights of expected oligomers for sE1E2.FD and sE1E2.CC are in parentheses. For molecular weight estimations, each N-glycan is approximated to be 2 kDa at each N×S/N×T sequon, a value within the molecular weight range of typical N-linked glycans. (FIG. 9B) X-ray structure of human c-Fos/c-Jun heterodimer (PDB code: 1FOS); only the coiled coil region that was used for the sE1E2.LZ scaffold is shown. c-Fos and c-Jun chains were colored to match the diagram of sE1E2.LZ. (FIG. 9C) X-ray structure of foldon domain (PDB code: 4NCU). All chains colored light blue to match the diagram for sE1E2.FD. (FIG. 9D) Model of CC1+CC2 heterohexameric peptide assembly. CC1 and CC2 chains colored to match the diagram for sE1E2.CC. All structures were visualized in PyMOL (Schrodinger).

FIG. 10 shows characterization of the peptide complex CC1+CC2. Shown are the chromatographic traces for the CC1+CC2 complex (blue line) and other tested designs (labeled HEX1-4) following elution from a Superdex 75 size exclusion chromatography column (Cytiva). The CC1+CC2 complex elutes at a volume consistent with hexameric assembly. Indicated on the chromatograph is the estimated molecular weight for CC1+CC2, calculated based on the retention volumes of molecular size standards (Bio-Rad).

FIG. 11 shows E1 and E2 western blots of sE1E2 supernatant. HCV1 antibody at 5 μg/ml was used for the E2 western blot. H-111 antibody at 10 μg/ml was used for the E1 western blot. All sE1E2 supernatant samples were loaded under reducing conditions. Supernatants were concentrated 10 times prior to E1 western blot. Molecular weights, in kDa, of the western blot markers closest to observed bands are indicated on the left. Expected band positions of E1, E2, and E1E2 are indicated with black triangles on right and labeled.

FIG. 12 shows Western blots of supernatant from E1-Jun/E2-Fos co-expression. sE1E2.LZ components E1-Jun and E2-Fos were co-expressed in trans, both with tPA signal sequence, then probed with HCV1 (anti-E2) or H-111 (anti-E1) antibody under reducing conditions. E1 and E2 detection was compared to expression levels of the full sE1E2.LZ construct. Supernatants were concentrated 10 times prior to E1 western blot. Molecular weights of the marker closest to observed bands are labeled. In both western blots, E1-Jun/E2-Fos and sE1E2.LZ were loaded in non-adjacent wells but were placed together to aid viewing.

FIG. 13 shows E1 and E2 western blots of sE1E2 cell lysate. HCV1 antibody at 5 ug/ml was used for the E2 western blot. H-111 antibody at 10 μg/ml was used for the E1 western blot. All sE1E2 lysate samples were loaded under reducing conditions. Supernatants were concentrated 10× prior to E1 western blot. Expected band sizes of E1, E2, and E1E2 are indicated with black triangles and labeled accordingly. E1 detection of sE1E2.R6 and sE1E2.CC were loaded in non-adjacent wells but are grouped together in this figure to aid comparisons.

FIG. 14 shows quantitative western blots comparing sE1E2.LZ supernatant and cell lysate. One μl of each sample was used for E2 probing with anti-E2 antibody HCV1 at a concentration of 5 μg/ml in separate western blots. Standards with defined amounts of purified sE2 protein (50, 100, or 200 ng) were included in each western blot, as shown in the figure. Band intensities of supernatant and cell lysate samples were compared with the standard curve to estimate protein amount via ImageQuant software (Cytiva) and the proportion of expressed sE1E2.LZ that was secreted in supernatant. E2 detection of sE1E2.LZ supernatant and cell lysate was aligned by molecular weight range of markers from separate western blots. sE1E2.LZ cell lysate and 50 ng of sE2 were loaded in non-adjacent wells but are grouped together in this figure to aid viewing.

FIGS. 15A-15D show size exclusion chromatography of sE1E2.LZ, sE1E2GS3, and mbE1E2. Chromatographic traces for (FIG. 15A) sE1E2.LZ and (FIG. 15D) mbE1E2 shown in blue lines plotted with molecular weight standards shown in grey lines after elution from a Superdex 200 size exclusion chromatography column (Cytiva). Molecular weight estimates for the center of each peak are labeled based on comparisons with elution of high molecular weight standards (Cytiva), with molecular masses of 670, 440, 158, 73, and 44 kDa. The range for elution fractions F1-F10 used for analysis is shown as a red line. Western blots of sE1E2.LZ for E2 (FIG. 15B), sE1E2.LZ for E1 (FIG. 15C), mbE1E2 for E2 (FIG. 15E), and mbE1E2 for E1 (F) under non-reducing conditions. HCV1 antibody was used to probe for E2, while H-111 antibody was used to probe for E1. Molecular weights, in kDa, of the western blot markers closest to observed bands are indicated on the left of each panel. All fractions had 250 ng loaded for improved visualization of size. For E1 western blots, all fractions were concentrated 10 times prior to loading. Putative E1 monomer, dimer, and trimer populations shown in panel (F) are highlighted with red initials.

FIG. 16 shows size exclusion chromatography of sE1E2GS3. Chromatographic trace of sE1E2GS3 shown as a blue line plotted with molecular weight standards shown as a grey line after elution from a Superdex 200 size exclusion chromatography column (Cytiva). The elution fractions F1-F9 used for subsequent analysis is shown as a red line. A molecular weight estimate for the center of the peak is labeled based on comparisons with elution of high molecular weight standards (Cytiva), with values of 670, 440, 158, 73, and 44 kDa.

FIG. 17 shows SDS-PAGE of mbE1E2, sE1E2.LZ, and sE1E2GS3 demonstrated relative purity of purified protein. Yield of each protein in μg per 100 ml of transfected cells is shown underneath the corresponding sample. 3.75 μg of protein was loaded for each purified protein. Expected band sizes of E1, E2, and E1E2 are indicated with black triangles and labeled accordingly. Molecular weight markers closest to observed bands are also indicated.

FIGS. 18A-18F show sE1E2GS3 fractions from size exclusion chromatography analyzed by SDS-PAGE and western blot. Fractions F1-F9 show a gradient of molecular weights following elution. SDS-PAGE results for sE1E2GS3 fractions under (FIG. 18A) reducing and (FIG. 18B) non-reducing conditions, with molecular weights of the marker labeled. Western blot of sE1E2GS3 fractions under (FIG. 18C) reducing and (FIG. 18D) non-reducing conditions probed with HCV1 (anti-E2) antibody. Molecular weights of the western blot marker closest to observed bands are indicated with black triangles and labeled. In FIG. 18C, the fraction with the highest concentration had 250 ng loaded, with other fractions scaled accordingly. In FIG. 18D, 250 ng of sE1E2GS3 fractions were loaded to improve visualization of size. Western blot of sE1E2GS3 fractions under (FIG. 18E) reducing and (FIG. 18F) non-reducing conditions probed with H-111 (anti-E1) antibody. Molecular weights of the western blot marker closest to observed bands are indicated with black triangles and labeled. All fractions were concentrated 10× prior to E1 western blots. In FIG. 18E, the fraction with the highest concentration had 250 ng loaded, with other fractions scaled accordingly. In FIG. 18F, 250 ng of sE1E2.LZ fractions were loaded to improve visualization of size.

FIGS. 19A-19D show sE1E2.LZ fractions from size exclusion chromatography analyzed by SDS-PAGE with stain-free detection (Bio-Rad) and western blot. Elution fractions F1-F10 show both E1 and E2 in SDS-PAGE under reducing conditions (FIG. 19A) and a molecular weight gradient in SDS-PAGE under non-reducing conditions ((FIG. 19B). Molecular weights in the protein ladder (Bio-Rad) for SDS-PAGE are indicated. Western blots of sE1E2.LZ fractions under reducing conditions when probed with HCV1 (anti-E2) antibody ((FIG. 19C) or H-111 (anti-E1) antibody ((FIG. 19D). Molecular weights of the western blot marker closest to observed bands are indicated with black triangles and labeled. In both western blots, the fraction with the highest concentration had 250 ng loaded, with other fractions scaled accordingly. For the E1 western blot, all fractions were concentrated 10 times prior to loading.

FIGS. 20A and 20B show mbE1E2 elution fractions from SEC analyzed by western blot under reducing conditions. Elution fractions were probed with HCV1 (anti-E2) antibody ((FIG. 20A) or H-111 (anti-E1) antibody ((FIG. 20B). Molecular weights of the western blot marker closest to observed bands are indicated with black triangles and labeled.

FIG. 21 show analysis of deglycosylation of purified mbE1E2, sE1E2.LZ, and sE2 by western blot under reducing (left) and non-reducing (right) conditions, with molecular weights of the marker labeled. 800 ng of each deglycosylated sample, along with a paired sample with intact glycans, were loaded in each lane of the reducing western blot. Some degradation of deglycosylated sE2 is apparent as the band intensity is markedly reduced. To aid detection of the full range of species present in the non-reducing western, an additional sample was added as needed. It is apparent that deglycosylation either allows separation or induces formation of additional species in the non-reducing western blot.

FIGS. 22A-22D show analytical characterization of sE1E2.LZ and mbE1E2 size and heterogeneity. AUC profiles of (FIG. 22A) purified sE1E2.LZ with or without detergent β-OG and (FIG. 22B) purified mbE1E2. Shown are the distribution of Lamm equation solutions c(s) for the two proteins (blue or black lines). Calculated sedimentation coefficients for the peaks are labeled. Observed species for sE1E2.LZ approximately correspond to a heterodimer at 4.9 S, a dimer of heterodimers at 7.7 S, and higher-order aggregates at 10.3 S. Observed species for mbE1E2 approximately correspond to free E2 at 4.0 S, a dimer of heterodimers at 6.6 S, a trimer of heterodimers at 9.1 S, and a tetramer of heterodimers and higher-order aggregates at >10 S. (FIG. 22C) sE1E2.LZ and (FIG. 22D) mbE1E2 characterization with SEC-MALS. The chromatographs of each protein are shown as blue lines. For reference, chromatographs of molecular weight standards are shown as grey lines in panels (FIG. 22C) and (FIG. 22D), corresponding to molecular masses of 670, 158, 44, 17, and 1.35 kDa. The MALS scattering sizes between the peak half-maxima are shown as red lines, with the estimated molecular weight at the center of each peak labeled, and size distribution of each range in parentheses. Based on calculated molecular weights of each heterodimer and SEC-MALS molecular size ranges, these peaks predominantly contain oligomers of (FIG. 22C) 1-2 sE1E2.LZ heterodimers and (FIG. 22D) 5-27 mbE1E2 heterodimers.

FIG. 23 shows comparison of mbE1E2 and sE1E2.LZ size and heterogeneity by blue native gel electrophoresis followed by western blot probed with either HCV1 (anti-E2) or H-111 (anti-E1) antibodies. E2 detection of mbE1E2 and sE1E2.LZ originated from different gels, which were then aligned to make the range of molecular weights equivalent. E1 detection of mbE1E2 and sE1E2.LZ was also conducted on separate gels, then aligned by molecular weight range.

FIG. 24 shows initial antigenicity screening of sE1E2 designs in ELISA. mbE1E2, sE1E2.LZ, sE1E2GS3, and sE2 were coated on ELISA plates at a concentration of 2 μg/ml and tested for binding to a panel of E2 and E1E2 bnAbs, representing E2 antigenic domains E (HCV1), B (AR3A), and D (HC84.26.WH.5DL), as well as E1E2 domains AR4 (AR4A) and AR5 (AR5A). Binding was measured at 450 nm with an antibody concentration of 0.185 μg/ml. Negative controls shown are an unrelated antibody (CA45) or PBS.

FIG. 25 shows measurement of binding to the CD81 receptor by surface plasmon resonance (SPR). CD81 binding kinetic curves to (A) mbE1E2, (B) sE1E2.LZ, and (C) sE2 are shown. Kinetic (kon, koff) and steady-state (Kd; calculated as koff/kon) binding parameters were calculated based on a 1:1 model and are shown in each panel.

FIGS. 26A-26C show immunogenicity assessment of sE2, mbE1E2, and sE1E2.LZ. Six mice per group were immunized with sE2, mbE1E2 or sE1E2.LZ, and sera were tested for binding to (FIG. 26A) mbE1E2 and (FIG. 26B) H77C-pseudotyped HCV pseudoparticles (HCVpp) in ELISA. One mouse in the sE2-immunized group died prior to final bleed, thus responses for five mice are shown for that group. Endpoint titers were calculated using Graphpad Prism, and geometric mean titers are shown for each group as black lines. (FIG. 26C) Neutralization of H77C HCVpp by immunized murine sera. Half-maximal inhibitory dose (ID50) values were calculated in Graphpad Prism for individual mice, and average ID50 titers for each immunized group are shown as black lines. The minimal serum dilution used for ID50 measurement (1:64) is shown as a horizontal dashed line, for reference. P-values between group endpoint titer or ID50 values were calculated using Kruskal-Wallis analysis of variance with Dunn's multiple comparison test (ns, not significant: p>0.05; *: p<0.05; **: p<0.01).

FIG. 27 shows data and calculated curves for H77C HCVpp neutralization by immunized (Day 56) murine sera. Data are shown for individual mice, and names (key on right) correspond to immunized groups (G1: mbE1E2, G2: sE1E2.LZ, G3: sE2), with six mice per group. Pooled pre-immune sera from each group were tested as controls. One mouse from G3 died prior to Day 56, thus had no serum available for testing. Serum dilutions (x-axis) are two-fold serial dilutions, starting at 1:64 (Reciprocal Serum Dilution=64).

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of aspects of the present disclosure which will be limited only by the appended claims.

It must be noted that as used herein and in the appended claims, the singular forms “a” “an”, and “the” include plural reference unless the context clearly dictates otherwise. Thus, for example, reference to “a glycoprotein” includes a plurality of such glycoproteins, reference to “the glycoprotein” is a reference to one or more glycoproteins and equivalents thereof known to those skilled in the art, and so forth.

The term “hepatitis C virus” or “HCV”, as used herein, refers to any one of a number of different genotypes and isolates of hepatitis C virus. Thus, “HCV” encompasses any of a number of genotypes, subtypes, or quasispecies, of HCV, including, but not limited to genotype 1, 2, 3, 4, 6, 7, 8, etc. and subtypes (e.g., 1a, 1b, 2a, 2b, 3a, 4a, 4c, etc.), and quasispecies. Representative HCV genotypes and isolates include, but are not limited to the H77 (genotype 1, subtype 1a), Con1 (genotype 1, subtype 1b), HC-J1 (genotype 1, subtype 1b), BK (genotype 1, subtype 1b), HC-J4 (genotype 1, subtype 1b), HC-JT (genotype 1, subtype 1b), HC-J6 (genotype 2, subtype 2a), HC-J8 (genotype 2, subtype 2b), NZL1 (genotype 3, subtype 3a), and JK049 (genotype 3, subtype 3k), ED43 (genotype 4, subtype 4a), SA13 (genotype 5, subtype 5a), EUHK2 (genotype 6, subtype 6a), QC69 (genotype 7, subtype 7a). A list of HCV genotypes/subtypes can be found at //talk.ictvonline.org/ictv_wikis/flaviviridae/w/sg_flavi/634/table-1---confirmed-hcv-genotypes-subtypes-may-2019.

As used herein, the term “subject” or “patient” can be used interchangeably and refer to any organism to which a protein or composition of examples of this disclosure may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

The term “percent (%) identity” can be used interchangeably herein with the term “percent (%) homology” and refers to the level of nucleic acid or amino acid sequence identity when aligned with a wild type sequence using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for any one of the inventive proteins, as described herein. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet. See also, Altschul, et al., 1990 and Altschul, et al., 1997. Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM62 matrix. (See, e.g., Altschul, S. F., et al., Nucleic Acids Res. 25:3389-3402, 1997.) A preferred alignment of selected sequences in order to determine “% identity” between two or more sequences, is performed using for example, the CLUSTAL-W program in Mac Vector version 13.0.7, operated with default parameters, including an open gap penalty of 10.0, an extended gap penalty of 0.1, and a BLOSUM30 similarity matrix.

Amino acid alterations such as substitutions, deletions, insertions or any combination thereof may be used to arrive at a final derivative, variant, or analog. Generally, these changes are done on a few nucleotides to minimize the alteration of the molecule. However, larger changes may be tolerated in certain circumstances.

Generally, the nucleotide identity between individual variant sequences can be at least 90%, 91%, 92%, 93%, 94%, 95%, 96%, 97%, 98%, 99%, or 100%. Thus, a “variant sequence” can be one with the specified identity to a parent or reference sequence (e.g. wild-type sequence) of examples of the present disclosure that comprise one or more amino acid alterations, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. In some aspects, a variant hepatitis C virus (HCV) E2 polypeptide can be one or more of the modified HCV E2 polypeptides disclosed herein. For example, a modified HCV E2 polypeptide can be a sequence that contains 1, 2, or 3, 4 amino acid base changes as compared to the parent or reference sequence of examples of the present disclosure, and shares or improves biological function, specificity and/or activity of the parent sequence. Thus, a modified HCV E2 polypeptide can be one with the specified identity to the parent sequence of the present disclosure, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of a reference sequence (e.g. wild-type sequence or E2 protein sequence).

The terms “variant” and “mutant” or “modified” can be used interchangeably. As used herein, the term “variant” refers to a modified nucleic acid or protein which displays the same characteristics when compared to a reference nucleic acid or protein sequence. A modified HCV E2 polypeptide can be at least 65, 70, 75, 80, 85, 90, 95, or 99 percent homologous to a reference sequence. In some aspects, a reference sequence can be a wild type HCV E2 glycoprotein nucleic acid sequence or a wild type HCV E2 glycoprotein protein sequence. Variants can also include nucleotide sequences that are substantially similar to sequences of E1 and E2 disclosed herein. A “variant” or “variant thereof” can mean a difference in some way from the reference sequence other than just a simple deletion of an N- and/or C-terminal amino acid residue or residues. Where the variant includes a substitution of an amino acid residue, the substitution can be considered conservative or non-conservative. Variants can include at least one substitution and/or at least one addition, there may also be at least one deletion. Variants can also include one or more non-naturally occurring residues.

As used herein an amino acid “substitution” refers to the replacement of one amino acid residue by a different amino acid residue. The substituted amino acid may be any of the 20 amino acids commonly found in human proteins, as well as atypical or non-naturally occurring amino acids. A substitution of an amino acid residue can be considered conservative or non-conservative. Conservative substitutions are those within the following groups: Ser, Thr, and Cys; Leu, ILe, and Val; Glu and Asp; Lys and Arg; Phe, Tyr, and Trp; and Gln, Asn, Glu, Asp, and His. In some aspects, the substitution can be a non-naturally occurring substitution. For example, the substitution may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂)ₙCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

As used herein, the term “wild-type” refers to a gene or protein which has the characteristics of that gene or protein when isolated from a naturally-occurring source. For example, a wild type HCV E2 polypeptide has the characteristics of the E2 polypeptide from a naturally occurring HCV genotype such as H77.

By “treat” is meant to administer a protein, nucleic acid, or composition of the present disclosure to a subject, such as a human or other mammal (for example, an animal model) in order to prevent or delay a worsening of the effects of a disease or condition, or to partially or fully reverse the effects of the disease or condition, For example, “treat” is meant to administer a protein, nucleic acid, or composition of the present disclosure to a subject, such as a human or other mammal (for example, an animal model) that has or has an increased susceptibility for developing infection with HCV or that has an infection with HCV, in order to prevent or delay a worsening of the effects of the HCV infection, or to partially or fully reverse the effects of the disease or condition.

By “prevent” is meant to minimize the chance that a subject who has an increased susceptibility for developing an infection with HCV actually develops the infection or disease or otherwise develops a cause of symptom thereof.

As used herein, the terms “administering” and “administration” refer to any method of providing a disclosed peptide, composition, or a pharmaceutical preparation to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed protein so as to treat a subject or induce an immune response. In an aspect, the skilled person can also alter or modify an aspect of an administering step so as to improve efficacy of a disclosed protein, nucleic acid, composition, or a pharmaceutical preparation.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present disclosure is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Modified Hepatitis C Virus (HCV) Glycoproteins

The HCV genome comprises a 5′-untranslated region that is followed by an open reading frame (ORF) that codes for about 3,010 amino acids. The ORF runs from nucleotide base pair 342 to 8,955 followed by another untranslated region at the 3′ end. The amino acids are subdivided into ten proteins in the order from 5′ to 3′ as follows: C; E1; E2; NS1; NS2; NS3; NS4 (a and b); and NS5 (a and b). These proteins are formed from the cleavage of the larger polyprotein by both host and viral proteases. The C, E1, and E2 proteins are structural and the NS1-NS5 proteins are nonstructural proteins. The C region codes for the core nucleocapsid protein. E1 and E2 are glycosylated envelope proteins that coat the virus. NS2 may be a zinc metalloproteinase. NS3 is a helicase. NS4a functions as a serine protease cofactor involved in cleavage between NS4b and NS5a. NS5a is a serine phosphoprotein whose function is unknown. The NS5b region has both RNA-dependent RNA polymerase and terminal transferase activity.

The envelope of HCV contains two glycoproteins, E1 and E2, that are encoded as part of the HCV polyprotein expressed in infected liver cells. This polyprotein is processed in the endoplasmic reticulum (ER) by signal peptidases and cellular glycosylation machinery to produce the mature E1E2 complex. These glycoproteins are membrane-anchored via their C-terminal transmembrane domains (TMDs), resulting in a membrane bound E1E2 (mbE1E2) complex.

Disclosed are modified HCV E1E2 glycoproteins. Disclosed are modified HCV E1E2 glycoproteins that do not comprise a transmembrane domain, and therefore can be secreted and are different in structure from the membrane bound E1E2 (mbE1E2).

Disclosed herein are modified HCV E1E2 glycoproteins that comprise E1 polypeptides and E2 polypeptides that can be from any HCV strain or genotype, including HCV genotype H77. With regard to the numbering and position of a particular mutation used herein, the numbering described herein refers to the numbering based on the HCV genotype H77. While other HCV genotypes may vary in sequence from the HCV strain H77, the positions of the disclosed amino acid alterations can be identified in any non-H77 HCV genotypes (and therefore non-H77 HCV E2 and E1E2 sequences) using tools such as those found at https://hcv.lanl.gov/content/sequence/NEWALIGN/align.html where a person of skill in the art, when provided with the information and guidance from the instant application can utilize the “H77 Coordinates”, as a means to identify and correlate the described positions (e.g. amino acid alterations) to specify the sites in non-H77 HCV sequences. For example, a person of skill in the art when provided with the information and guidance from the instant application can utilize the “H77 Coordinates”, to identify the amino acid positions corresponding to HCV genotype H77 amino acid positions 445, 632, and 634 in other HCV genotype amino acid sequences.

1. Secreted E1E2 Glycoproteins

Disclosed are modified HCV E1E2 glycoproteins comprising a HCV E1 polypeptide; a first scaffold element; a HCV E2 polypeptide; and a second scaffold element, wherein the HCV E1 polypeptide does not comprise a transmembrane domain, and wherein the HCV E2 polypeptide does not comprise a transmembrane domain. In some aspects, the absence of transmembrane domains allows the modified HCV E1E2 glycoproteins to be secreted.

In some aspects, the modified HCV E1E2 glycoproteins disclosed herein can comprise the sequence of:

(SEQ ID NO: 5)
YQVRNSSGLYHVTNDCPNSSIVYEAADAILHTPGCVPCVREGNASRCWVA
VTPTVATRDGKLPTTQLRRHIDLLVGSATLCSALYVGDLCGSVFLVGQLF
TFSPRRHWTTQDCNCSIYPGHITGHRMAWDMMMNWSPTAALVVAQLLRIP
QAIMDMIAPGGRIARLEEKVKTLKAQNSELASTANMLREQVAQLKQKVMN
YRRRRRRETHVTGGSAGRTTAGLVGLLTPGAKQNIQLINTNGSWHINSTA
LNCNESLNTGWLAGLFYQHKFNSSGCPERLASCRRLTDFAQGWGPISYAN
GSGLDERPYCWHYPPRPCGIVPAKSVCGPVYCFTPSPVVVGTTDRSGAPT
YSWGANDTDVFVLNNTRPPLGNWFGCTWMNSTGFTKVCGAPPCVIGGVGN
NTLLCPTDCFRKHPEATYSRCGSGPWITPRCMVDYPYRLWHYPCTINYTI
FKVRMYVGGVEHRLEAACNWTRGERCDLEDRDRSELSPLLLSTTQWQVLP
CSFTTLPALSTGLIHLHQNIVDVQYLYGVGSSIASWAI
PGGLTDTLQAETDQLEDKKSALQTEIANLLKEKEKLEFILAAYhhhhhh.

The HCV E1 polypeptide is shown with no markings. A first scaffold element, c-Jun, is shown in bold. A furin cleavage site, RRRRRR (SEQ ID NO: 12), is shown in italics. The HCV E2 polypeptide is shown in underline. A second scaffold element, c-fos, is shown in double underline. A purification tag (histidine tag), hhhhhh (SEQ ID NO:59), is shown in lowercase letters.

i. HCV E1 and E2 Polypeptides

Disclosed herein are modified HCV E1E2 glycoproteins comprising a HCV E1 polypeptide. In some aspects, the HCV E1 polypeptide is an ectodomain. In some aspects, the HCV E1 polypeptide comprises an ectodomain. In some aspects, the HCV E1 polypeptide consists of an ectodomain.

In some aspects, the HCV E1 polypeptide comprises the sequence of YQVRNSSGLYHVTNDCPNSSIVYEAADAILHTPGCVPCVREGNASRCWVAVTPTVA TRDGKLPTTQLRRHIDLLVGSATLCSALYVGDLCGSVFLVGQLFTFSPRRHWTTQDC NCSIYPGHITGHRMAWDMMMNWSPTAALVVAQLLRIPQAIMDMIA (SEQ ID NO:1). SEQ ID NO:1 is amino acids 192-349 of wild type H77 HCV (NCBI Accession No. NP_671491.1; Genbank AF009606).

Disclosed herein are modified HCV E1E2 glycoproteins comprising a HCV E2 polypeptide. In some aspects, the HCV E2 polypeptide is an ectodomain. In some aspects, the HCV E2 polypeptide comprises an ectodomain. In some aspects, the HCV E2 polypeptide consists of an ectodomain.

In some aspects, the HCV E2 polypeptide comprises the sequence of ETHVTGGSAGRTTAGLVGLLTPGAKQNIQLINTNGSWHINSTALNCNESLNTGWLAG LFYQHKFNSSGCPERLASCRRLTDFAQGWGPISYANGSGLDERPYCWHYPPRPCGIV PAKSVCGPVYCFTPSPVVVGTTDRSGAPTYSWGANDTDVFVLNNTRPPLGNWFGCT WMNSTGFTKVCGAPPCVIGGVGNNTLLCPTDCFRKHPEATYSRCGSGPWITPRCMV DYPYRLWHYPCTINYTIFKVRMYVGGVEHRLEAACNWTRGERCDLEDRDRSELSPL LLSTTQWQVLPCSFTTLPALSTGLIHLHQNIVDVQYLYGVGSSIASWAI (SEQ ID NO:2). SEQ ID NO:2 is amino acids 384-714 of wild type H77 HCV.

In some aspects, HCV E1 or E2 polypeptides are any HCV E1 or E2 polypeptide having at least about 70, 75, 80, 85, 90, 95, 99, or 100% identity, to a wild type HCV E1 or E2 polypeptide, respectively, from any of the known HCV genotypes and/or subtypes. For example, disclosed are modified HCV E1 or E2 polypeptides having at least about 70, 75, 80, 85, 90, 95, or 100% identity to the E1 or E2 polypeptides of the H77 (Genbank AF009606) genotype of HCV, respectively. In some aspects, HCV E1 polypeptides can be any HCV E1 polypeptide having at least about 70, 75, 80, 85, 90, 95, 99, or 100% identity to SEQ ID NO:1. In some aspects, HCV E2 polypeptides can be any HCV E2 polypeptide having at least about 70, 75, 80, 85, 90, 95, 99, or 100% identity to SEQ ID NO:2. Thus, disclosed are variants of HCV E1 and E2 polypeptides.

In some aspects, the disclosed modified HCV E1E2 glycoproteins comprising a HCV E1 polypeptide and a HCV E2 polypeptide can be formed by co-expressing a HCV E1 polypeptide and a HCV E2 polypeptide in trans, both having a scaffold element on the C-terminal end which helps bring them together to form a scaffold and the modified HCV E1E2 glycoprotein. In some aspects, the HCV E1 polypeptide and the HCV E2 polypeptide can be expressed as a single polypeptide including the first and second scaffold elements.

ii. Scaffold

In some aspects, a modified HCV E1E2 glycoprotein can comprise a HCV E1 polypeptide, wherein the HCV E1 polypeptide does not comprise a transmembrane domain; a scaffold, wherein the scaffold comprises a first scaffold element and a second scaffold element; and a HCV E2 polypeptide, wherein the HCV E2 polypeptide does not comprise a transmembrane domain. In some aspects, the first scaffold element and second scaffold element are capable of interacting with each other forming a scaffold. In some aspects, the scaffold, and thus the scaffold elements, can be necessary for E1E2 assembly.

In some aspects the first scaffold element and a second scaffold element of the disclosed modified HCV E1E2 glycoproteins can be in any order. Thus, in some aspects, the first scaffold element can be located on the C-terminus of the HCV E1 polypeptide and the second scaffold element can be located on the C-terminus of the HCV E2 polypeptide. In other instances, the first scaffold element can be located on the C-terminus of the HCV E2 polypeptide and the second scaffold element can be located on the C-terminus of the HCV E1 polypeptide.

In some aspects, the first or second scaffold element can be the full sequence of c-Jun or c-Fos. In some aspects, the first scaffold element of a modified HCV E1E2 glycoprotein can be a subsequence of c-Jun and the second scaffold element of the modified HCV E1E2 glycoprotein can be a subsequence of c-Fos. As used herein a subsequence refers to a sequence (e.g. nucleic acid or amino acid) that comprises less than the full sequence of the referenced nucleic acid or amino acid sequence. In some aspects, when “subsequence of c-Jun” or “subsequence of c-Fos” is used in reference to the first or second scaffold element, the subsequence comprises a sequence necessary to form a leucine zipper. In some aspects, the first scaffold element is a subsequence of c-Fos and the second scaffold element is a subsequence of c-Jun. Thus, the first and second scaffold elements of the disclosed modified HCV E1E2 glycoproteins can be reversed in the location they are found on the E1E2 glycoprotein as long as they still retain the ability to interact with each other, thus forming a scaffold. For example, the first scaffold and second scaffold can be capable of forming a leucine zipper. In an aspect, c-Jun and c-fos can interact with each other to form a leucine zipper.

In some aspects, the subsequence of c-Jun is RIARLEEKVKTLKAQNSELASTANMLREQVAQLKQKVMNY (SEQ ID NO:8). In some aspects, the c-Fos subsequence is

(SEQ ID NO: 9)
LTDTLQAETDQLEDKKSALQTEIANLLKEKEKLEFILAAY.

In some aspects, one or both of the c-Jun and c-Fos sequences can have a linker. In some aspects, the linker can be PGG. For example, the subsequence of c-Jun can be PGGRIARLEEKVKTLKAQNSELASTANMLREQVAQLKQKVMNY (SEQ ID NO:10) and/or the subsequence of c-Fos can be

(SEQ ID NO: 11)
PGGLTDTLQAETDQLEDKKSALQTEIANLLKEKEKLEFILAAY.

In some aspects, a scaffold of the disclosed modified HCV E1E2 glycoproteins can be composed of a single scaffold element, such as a foldon-based scaffold. When the scaffold is a single scaffold element, the scaffold (and thus single scaffold element) can be located on either the HCV E1 or E2 polypeptide. Thus, in some aspects, a modified HCV E1E2 glycoprotein can comprise a HCV E1 polypeptide, wherein the HCV E1 polypeptide does not comprise a transmembrane domain; a scaffold; and a HCV E2 polypeptide, wherein the HCV E2 polypeptide does not comprise a transmembrane domain. In some aspects, the scaffold can be present on the HCV E1 polypeptide or the HCV E2 polypeptide. For example, the scaffold can be a foldon-based scaffold. Thus, in some aspects scaffold elements are present on each of the HCV E1 polypeptide and the HCV E2 polypeptide, which can result in a scaffold, and in some aspects the HCV E1E2 glycoprotein comprises a scaffold on only one of the HCV E1 polypeptide or the HCV E2 polypeptide.

In some aspects, the first scaffold of a modified HCV E1E2 glycoprotein can be a first coiled-coil domain and the second scaffold is a second coiled-coil domain. In such an arrangement, the interaction of the first and second coiled-coil domains can provide a scaffold for the HCV E1E2 glycoprotein. In some aspects, a first or second coiled-coil domain can comprise the sequence of AAEDLLELAHTILKTARNQLRTMEILRKER (SEQ ID NO:3). In some aspects, if a first or second coiled-coil domain comprises SEQ ID NO:3, then the opposite coiled-coil domain can comprise the sequence of ADERRKAKELLKEAEEIWKRINELAERETK (SEQ ID NO:4). In such an arrangement, if the first coiled-coil domain is SEQ ID NO:3 then the second coiled-coil domain can be SEQ ID NO:4 or if the first coiled-coil domain is SEQ ID NO:4 then the second coiled-coil domain can be SEQ ID NO:3.

In some aspects, the first scaffold element and second scaffold element are not transmembrane domains. Thus, the E1E2 assembly is not due to the location of HCV E1 polypeptide in a cell membrane close to HCV E2 polypeptide.

iii. Cleavage Site

In some aspects, the disclosed modified HCV E1E2 glycoproteins can further comprise a cleavage site. For example, disclosed are modified HCV E1E2 glycoproteins comprising a HCV E1 polypeptide, wherein the HCV E1 polypeptide does not comprise a transmembrane domain; a first scaffold element; a cleavage site; a HCV E2 polypeptide, wherein the HCV E2 polypeptide does not comprise a transmembrane domain; and a second scaffold element.

In some aspects, the cleavage site is located between the HCV E1 polypeptide and the HCV E2 polypeptide. In some aspects, the cleavage site can be located after the first scaffold element and before the HCV E2 polypeptide.

In some aspects, the cleavage site can be a furin cleavage site. In some aspects, the furin cleavage site comprises six arginines (RRRRRR; SEQ ID NO: 12). In some aspects, other furin cleavage sites can be RRRRKR (SEQ ID NO:13) or RRRKKR (SEQ ID NO:14). In some aspects, the furin cleavage site is R-X-K/R-R (SEQ ID NO:15/16). In some aspects, the furin cleavage site can be, but is not limited to Tobacco Etch Virus (TEV) protease cleavage site (ENLYFQS; SEQ ID NO:17) or human rhinovirus type 14 (HRV) 3C protease cleavage site (LEVLFQGP; SEQ ID NO:18).

In some aspects, the cleavage site can be present when the modified HCV E1E2 glycoprotein is expressed as a single polypeptide. The cleavage site can then be used to cleave the HCV E1 polypeptide from the HCV E2 polypeptide which would allow the HCV E1 polypeptide and the HCV E2 polypeptide to come together via the scaffold (e.g. first scaffold element and second scaffold element) correctly assembling the E1E2 glycoprotein.

In some aspects, the disclosed modified HCV E1E2 glycoproteins do not comprise a cleavage site. For example, in some aspects, if the HCV E1 polypeptide and the HCV E2 polypeptide of a modified HCV E1E2 glycoprotein are co-expressed in trans then a cleavage site may not be necessary.

iv. Leader Sequence

In some aspects, the disclosed modified HCV E1E2 glycoproteins can further comprise a leader sequence at the N-terminal end of the HCV E1 polypeptide. For example, disclosed are modified HCV E1E2 glycoproteins comprising a HCV E1 polypeptide, wherein the HCV E1 polypeptide does not comprise a transmembrane domain, wherein the HCV E1 polypeptide comprises a leader sequence at the N-terminal end of the HCV E1 polypeptide; a first scaffold element; a HCV E2 polypeptide, wherein the HCV E2 polypeptide does not comprise a transmembrane domain; and a second scaffold element.

In some aspects, the leader sequence can be a tissue plasminogen activator (tPA) leader sequence. In some aspects, the leader sequence can be derived from human IL-2 or murine Ig-kappa. Table 3 shows examples, but is not an exclusive list, of leader sequences that can be used in the compositions disclosed herein.

TABLE 3

| Leader sequence Name | Sequence |
|---|---|
| Human OSM | MGVLLTQRTLLSLVLALLFPSMASM (SEQ ID NO: 19) |
| VSV-G | MKCLLYLAFLFIGVNC (SEQ ID NO: 20) |
| Mouse Ig Kappa | METDTLLLWVLLLWVPGSTGD (SEQ ID NO: 21) |
| Human IgG2 H | MGWSCIILFLVATATGVHS (SEQ ID NO: 22) |
| BM40 | MRAWIFFLLCLAGRALA (SEQ ID NO: 23) |
| Secrecon | MWWRLWWLLLLLLLLWPMVWA (SEQ ID NO: 24) |
| Human IgKVIII | MDMRVPAQLLGLLLLWLRGARC (SEQ ID NO: 25) |
| CD33 | MPLLLLLPLLWAGALA (SEQ ID NO: 26) |
| tPA | MDAMKRGLCCVLLLCGAVFVSPS (SEQ ID NO: 27) |
| Human Chymotrypsinogen | MAFLWLLSCWALLGTTFG (SEQ ID NO: 28) |
| Human trypsinogen-2 | MNLLLILTFVAAAVA (SEQ ID NO: 29) |
| Human IL-2 | MYRMQLLSCIALSLALVTNS (SEQ ID NO: 30) |
| Gaussia luc | MGVKVLFALICIAVAEA (SEQ ID NO: 31) |
| Albumin(HSA) | MKWVTFISLLFSSAYS (SEQ ID NO: 32) |
| Influenza Haemagglutinin | MKTIIALSYIFCLVLG (SEQ ID NO: 33) |
| Human insulin | MALWMRLLPLLALLALWGPDPAAA (SEQ ID NO: 34) |
| Silkworm Fibroin LC | MKPIFLVLLVVTSAYA (SEQ ID NO: 35) |

v. Other Moieties

In some aspects, additional sequences that aid in solubilizing, detecting, and/or purifying the HCV E1E2 glycoprotein can be added to one or more elements of the disclosed glycoproteins. In some aspects, the disclosed modified HCV E1E2 glycoproteins can further comprise a detectable label or diagnostic moiety.

In some aspects, the disclosed modified HCV E1E2 glycoproteins can further comprise a detectable moiety. In some aspects, the detectable moiety can be located at the C-terminal end of the second scaffold element. For example, disclosed are modified HCV E1E2 glycoproteins comprising a HCV E1 polypeptide, wherein the HCV E1 polypeptide does not comprise a transmembrane domain; a first scaffold element; a HCV E2 polypeptide, wherein the HCV E2 polypeptide does not comprise a transmembrane domain; and a second scaffold element, wherein the second scaffold element comprises a detectable moiety.

In some aspects, the detectable moiety is a purification tag or a label. As used herein, a detectable moiety, is any molecule that can be associated with a HCV E1 polypeptide, HCV E2 polypeptide, a first scaffold element, or a second scaffold element, directly or indirectly, and which results in a measurable, detectable signal, either directly or indirectly. Many such detectable moieties are known to those of skill in the art. Examples of detectable moieties can be, but are not limited to, radioactive isotopes, fluorescent molecules, phosphorescent molecules, enzymes, antibodies, and ligands.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like. Other labels can include biotin, streptavidin, horseradish peroxidase, or luciferase.

In some aspects, the disclosed modified HCV E1E2 glycoproteins can further comprise a maltose binding protein sequence. A maltose binding protein can help with protein solubilization, protein detection, and protein purification. In some aspects, the disclosed modified HCV E1E2 glycoproteins can further comprise a histidine tag. A histidine tag can be used for protein purification and detection. Those of skill in the art would understand those known sequences available for solubilizing, detecting, and/or purifying polypeptides that can be used with the disclosed modified HCV E1E2 glycoproteins.

In an exemplary embodiment, carrier proteins represented by virus capsid proteins that have the capability to self-assemble into virus-like particles (VLPs) are utilized in combination with the disclosed modified HCV E1E2 glycoproteins. Examples of VLPs used as peptide carriers are hepatitis B virus surface antigen and core antigen, hepatitis E virus particles, polyoma virus, bovine papilloma virus, and the like.

In another embodiment, the disclosed modified HCV E1E2 glycoproteins can be coupled to one of a number of carrier molecules, known to those of skill in the art. A carrier protein should be of sufficient size for the immune system of the subject to which it is administered to recognize its foreign nature and develop antibodies to it.

In some cases a carrier molecule can be directly coupled to the disclosed modified HCV E1E2 glycoproteins. In other cases, there is a linker molecule inserted between the carrier molecule and the disclosed modified HCV E1E2 glycoproteins. For example, the coupling reaction may require a free sulfhydryl group on the peptide. In such cases, an N-terminal cysteine residue is added to the modified HCV E1E2 glycoprotein when the modified HCV E1E2 glycoprotein is synthesized. In an exemplary embodiment, traditional succinimide chemistry is used to link the modified HCV E1E2 glycoprotein to a carrier protein. Methods for preparing such modified HCV E1E2 glycoprotein:carrier protein conjugates are generally known to those of skill in the art and reagents for such methods are commercially available (e.g., from Sigma Chemical Co.). Generally about 5-30 modified HCV E1E2 glycoprotein molecules are conjugated per molecule of carrier protein.

Any of the disclosed modified HCV E1E2 glycoproteins can be combined with other viral subunits to form an attenuated live virus or replication-defective virus. In some aspects, the disclosed modified HCV E1E2 glycoproteins can be combined with other elements to form nanoparticles carrying the disclosed modified HCV E1E2 glycoproteins.

2. Secreted HCV E1E2 Glycoprotein with Modified E2 Polypeptide

In some aspects, the disclosed modified HCV E1E2 glycoproteins can be combined with one or more of the modifications to HCV E2 polypeptide as described in U.S. Pat. No. 9,732,121, which is hereby incorporated by reference in its entirety for its teaching of modifications to HCV E2 polypeptide.

Disclosed are modified HCV E1E2 glycoproteins comprising a modified HCV E2 polypeptide. In some aspects, modified HCV E1E2 glycoproteins comprise an altered or mutated E2 polypeptide. A modified HCV E2 polypeptide can be any HCV E2 polypeptide that is not 100% identical to the corresponding amino acids of any wild type strain HCV E2 polypeptide. As described herein, the modified HCV E1E2 glycoproteins comprise an HCV E1 polypeptide and a HCV E2 polypeptide, wherein the HCV E1 and E2 polypeptides do not comprise a transmembrane domain. Thus, in some aspects, the disclosed modified HCV E1E2 glycoproteins are secreted.

Disclosed are modified HCV E1E2 glycoproteins comprising a HCV E1 polypeptide; a first scaffold element; a modified HCV E2 polypeptide; and a second scaffold element, wherein the HCV E1 polypeptide does not comprise a transmembrane domain; a first scaffold element, wherein the modified HCV E2 polypeptide does not comprise a transmembrane domain, wherein the modified HCV E2 polypeptide comprises an antigenic domain D, and wherein the modified HCV E2 polypeptide comprises one or more amino acid alterations in the antigenic domain D.

i. HCV E1 Polypeptide and Modified HCV E2 Polypeptide

Disclosed herein are modified HCV E1E2 glycoproteins comprising a HCV E1 polypeptide. In some aspects, the HCV E1 polypeptide is an ectodomain. In some aspects, the HCV E1 polypeptide comprises an ectodomain. In some aspects, the HCV E1 polypeptide consists of an ectodomain.

In some aspects, the HCV E1 polypeptide comprises the sequence of YQVRNSSGLYHVTNDCPNSSIVYEAADAILHTPGCVPCVREGNASRCWVAVTPTVA TRDGKLPTTQLRRHIDLLVGSATLCSALYVGDLCGSVFLVGQLFTFSPRRHWTTQDC NCSIYPGHITGHRMAWDMMMNWSPTAALVVAQLLRIPQAIMDMIA (SEQ ID NO:1). SEQ ID NO:1 is amino acids 192-349 of wild type H77 HCV (NCBI Accession No. NP_671491.1; GenbankAF009606). In some aspects, HCV E1 polypeptides are any HCV E1 polypeptide having at least about 70, 75, 80, 85, 90, 95, 99, or 100% identity, to a wild type HCV E1 polypeptide from any of the known HCV genotypes and/or subtypes. For example, disclosed are HCV E1 polypeptides having at least about 70, 75, 80, 85, 90, 95, or 100% identity to the E1 polypeptides of the H77 (Genbank AF009606) genotype of HCV. In some aspects, HCV E1 polypeptides are any HCV E1 polypeptide having at least about 70, 75, 80, 85, 90, 95, 99, or 100% identity to SEQ ID NO:1. Thus, disclosed are variants of HCV E1 polypeptides.

Disclosed herein are modified HCV E1E2 glycoproteins comprising a modified HCV E2 polypeptide. In some aspects, the modified HCV E2 polypeptide is an ectodomain. In some aspects, the modified HCV E2 polypeptide comprises an ectodomain. In some aspects, the HCV E2 polypeptide consists of an ectodomain.

The disclosed modified HCV E1E2 glycoproteins can comprise a HCV E2 polyprotein having at least about 70, 75, 80, 85, 90, 95, or 99% identity, but not 100% identity, to a wild type HCV E2 polypeptide from any of the known HCV genotypes and/or subtypes and comprising one or more amino acid alterations in the antigenic domain D. In some aspects, because the HCV E2 polypeptide is not 100% identical to a wild type HCV E2 polypeptide, the polypeptide can be referred to as a modified HCV E2 polypeptide. For example, disclosed are modified HCV E2 polypeptides having at least about 70, 75, 80, 85, 90, 95, or 99% identity, but not 100% identity, to the H77 (Genbank AF009606) genotype of HCV and comprising one or more amino acid alterations in the antigenic domain D. Thus, disclosed are variants of HCV E2 polypeptides.

In some instances, a modified HCV E2 glycoprotein can have at least about 70, 75, 80, 85, 90, 95, or 99% identity, but not 100% identity, to amino acid residues 384-714 of NCBI Accession No. NP_671491.1 (HCV strain H77). Thus, in some aspects, the HCV E2 polyprotein comprises an amino acid sequence with 70% identity to SEQ ID NO:2. In some aspects, disclosed are modified HCV E2 glycoproteins comprising an amino acid sequence with at least about 70, 75, 80, 85, 90, 95, or 99% identity to SEQ ID NO:2 and comprising one or more amino acid alterations in the antigenic domain D.

Disclosed herein are modified HCV E1E2 glycoproteins comprising a modified HCV E2 polypeptide. In some aspects, a modified HCV E2 polypeptide is a HCV E2 polypeptide comprising everything except the transmembrane domain. In some aspects, a modified HCV E2 polypeptide is a HCV E2 polypeptide comprises everything except the transmembrane domain and comprises one or more amino acid alterations in the antigenic domain D. In some aspects, a modified HCV E2 polypeptide can have a length of from about 200 amino acids (aa) to about 250 aa, from about 250 aa to about 275 aa, from about 275 aa to about 300 aa, from about 300 aa to about 325 aa, from about 325 aa to about 350 aa, or from about 350 aa to about 365 aa. In some aspects, a modified HCV E2 glycoprotein can have a length of from about 200 amino acids (aa) to about 250 aa, from about 250 aa to about 275 aa, from about 275 aa to about 300 aa, from about 300 aa to about 325 aa, from about 325 aa to about 350 aa, or from about 350 aa to about 365 aa and comprising one or more amino acid alterations in the antigenic domain D.

Disclosed herein are modified HCV E1E2 glycoproteins comprising a modified HCV E2 polypeptide, wherein the modified HCV E2 polypeptides comprises an antigenic domain D, wherein the modified HCV E2 polypeptides comprise one or more amino acid alterations in the antigenic domain D. In some aspects, an amino acid alteration can be an amino acid substitution, deletion, or addition.

a. Proline Substitution

Disclosed herein are modified HCV E1E2 glycoproteins comprising a modified HCV E2 polypeptide comprising an antigenic domain D, wherein the modified HCV E2 glycoprotein comprises one or more amino acid alterations in the antigenic domain D. In some aspects, an amino acid alteration is an amino acid substitution. Disclosed are modified HCV E2 polypeptides comprising an antigenic domain D, wherein the modified HCV E2 polypeptides comprise one or more amino acid alterations in the antigenic domain D, wherein at least one amino acid alteration is a proline substitution. In some aspects, the proline substitution stabilizes an antibody-bound conformation of the antigenic domain D.

As provided herein, disclosed are modified HCV E2 glycoproteins comprising an antigenic domain D, wherein the modified HCV E2 glycoproteins comprise one or more amino acid alterations in the antigenic domain D, wherein at least one amino acid alteration is a proline substitution. In some aspects, the proline substitution occurs at position 445 based on the amino acid numbering of HCV strain H77. For example, a proline substitution at position 445 based on the amino acid numbering of HCV strain H77 is equivalent to a proline substitution at position 445 of strain JFH-1 (genotype 2a), which is an asparagine residue, or position 445 of strain S52 (genotype 3a), which is a histidine residue. However, in some aspects, position 445 based on the amino acid numbering of HCV strain H77 can be equivalent to a position different than 445 in a different strain or genotype. In some aspects, a proline substitution at position 445 based on the amino acid numbering of HCV strain H77 is equivalent to a proline substitution at position 62 of SEQ ID NO:2, which is the first 331 amino acids of the H77 E2 amino acid sequence. Position 445 is based on the full genomic polyprotein sequence of H77 whereas position 62 is based on just the HCV E2 glycoprotein amino acid sequence of SEQ ID NO:2. In some aspects, the proline substitution is a substitution of histidine (at position 445 of H77 or at a position corresponding with position 445 of H77) with proline. In other words, in some aspects, the proline substitution corresponds to an H445P substitution in wild type H77 HCV full polypeptide sequence. In some aspects, the proline substitution is a substitution of asparagine, arginine, or tyrosine (at a position corresponding with position 62 of the HCV E2 glycoprotein amino acid sequence of H77) with proline. In some aspects, the proline substitution is a substitution of any amino acid (at a position corresponding with position 445 of H77) with proline. In other words, in some aspects, the proline substitution corresponds to an H62P substitution in SEQ ID NO:2.

Disclosed herein are modified HCV E1E2 glycoproteins comprising a modified HCV E2 polypeptide wherein the modified HCV E2 glycoprotein comprises the sequence of SEQ ID NO:6. In some aspects, the modified HCV E2 glycoprotein consists of the sequence of SEQ ID NO:6. SEQ ID NO:6 is the H77 E2 glycoprotein, minus the transmembrane domain, comprising a H445P substitution (also referred to as a H62P substitution if basing on the HCV E2 sequence) as shown below: ETHVTGGSAGRTTAGLVGLLTPGAKQNIQLINTNGSWHINSTALNCNESLNTGWLAG LFYQPKFNSSGCPERLASCRRLTDFAQGWGPISYANGSGLDERPYCWHYPPRPCGIV PAKSVCGPVYCFTPSPVVVGTTDRSGAPTYSWGANDTDVFVLNNTRPPLGNWFGCT WMNSTGFTKVCGAPPCVIGGVGNNTLLCPTDCFRKHPEATYSRCGSGPWITPRCMV DYPYRLWHYPCTINYTIFKVRMYVGGVEHRLEAACNWTRGERCDLEDRDRSELSPL LLSTTQWQVLPCSFTTLPALSTGLIHLHQNIVDVQYLYGVGSSIASWAI (SEQ ID NO:6). A H62P substitution is shown in bold (which corresponds to H445P when numbering is based on H77 full polypeptide sequence).

Disclosed herein are modified HCV E1E2 glycoproteins comprising a modified HCV E2 polypeptide wherein the modified HCV E2 glycoprotein comprises a sequence with 70, 75, 80, 85, 90, 95, or 99% identity to SEQ ID NO:6, wherein the sequence comprises a H62P substitution as compared to SEQ ID NO:6. In other words, the modified HCV E2 glycoprotein comprises a sequence with 70, 75, 80, 85, 90, 95, or 99% identity to SEQ ID NO:2, wherein the sequence comprises at least the H62P substitution. Thus, the 70, 75, 80, 85, 90, 95, or 99% identity can be based on an alteration somewhere other than position 62 of SEQ ID NO:2. In some aspects, modified HCV E2 glycoproteins are disclosed comprising at least a proline substitution at position 62 of E2 (or 445 of HCV strain H77) as compared to SEQ ID NO:2.

In some aspects, the antigenic domain D of the modified HCV E2 polypeptide retains the ability to bind to an antibody specific to the antigenic domain D. For example, the H62P mutation present in SEQ ID NO:6 retains the ability of the modified HCV E2 polyprotein to bind to an antibody specific to the antigenic domain D. In some aspects, the antibody specific to the antigenic domain D is HC84.1 or HC84.26. Therefore, in some aspects the antigenic domain D of a modified HCV E2 glycoprotein retains the ability to bind to HC84.1 or HC84.26.

In some aspects, the modified HCV E2 polypeptides disclosed herein comprise an amino acid alteration in the antigenic D domain, wherein the amino acid alteration is a deletion of amino acids 384-407 as compared to wild type H77. In some aspects, the modified HCV E2 polypeptides disclosed herein comprise an amino acid alteration in the antigenic D domain, wherein the amino acid alteration is a deletion of amino acids 384-407 as compared to wild type H77 and further comprise a proline substitution disclosed herein. For example, disclosed herein are modified HCV E2 polypeptides comprising an antigenic domain D, wherein the modified HCV E2 glycoprotein comprises one or more amino acid alterations in the antigenic domain D, wherein the amino acid alteration in the antigenic D domain is a deletion of amino acids 384-407, wherein the modified HCV E2 polypeptide further comprises a H445P substitution as compared to wild type H77.

In some aspects, the modified HCV E2 glycoproteins disclosed herein are soluble. In some aspects, the soluble portion of the modified E2 glycoprotein of H77 is residues 384-661 of SEQ ID NO:1.

b. N-Glycan Sequon Substitution

N-glycosylation functions by modifying appropriate asparagine residues of proteins with oligosaccharide structures, thus influencing their properties and bioactivities. In some aspects, the disclosed modified HCV E2 polypeptides comprise an N-glycosylation in their antigenic domain A which blocks or decreases binding of antibodies to the antigenic domain A. In some aspects, the decrease in binding of antibodies to antigenic domain A of HCV E2 polypeptide can result in an increased binding to antigenic domain D which can provide a neutralizing effect.

Disclosed are modified HCV E1E2 glycoproteins comprising a HCV E1 polypeptide; a first scaffold element; a modified HCV E2 polypeptide; and a second scaffold element, wherein the HCV E1 polypeptide does not comprise a transmembrane domain; a first scaffold element, wherein the modified HCV E2 polypeptide does not comprise a transmembrane domain, wherein the modified HCV E2 polypeptide comprises an antigenic domain D, and wherein the modified HCV E2 polypeptide comprises one or more amino acid alterations in the antigenic domain D, wherein the modified HCV E2 polypeptide comprises an antigenic domain A, wherein the antigenic domain A comprises an N-glycan sequon substitution.

Disclosed are modified HCV E1E2 glycoproteins comprising a HCV E1 polypeptide, a first scaffold, a HCV E2 polypeptide, and a second scaffold, wherein the HCV E1 polypeptide does not comprise a transmembrane domain, wherein the HCV E2 polypeptide does not comprise a transmembrane domain, wherein the HCV E2 polypeptide comprises an antigenic domain A, and wherein the antigenic domain A comprises an N-glycan sequon substitution.

Thus, in some aspects, disclosed are modified HCV E1E2 glycoproteins comprising an alteration in the antigenic domain D, an N-glycan sequon substitution in the antigenic domain A, or both.

An N-glycan sequon is a sequence of consecutive amino acids in a protein that can serve as the attachment site for an N-glycan. In some aspects, the N-glycan sequon substitution is in the antigenic domain A of SEQ ID NO:2. In some aspects, the N-glycan sequon substitution is in the antigenic domain A of an amino acid sequence with 70, 75, 80, 85, 90, 95 or 99% identity to SEQ ID NO:2.

In some aspects, the N-glycan sequon substitution results in an Asn-Xaa-Ser or Asn-Xaa-Thr substitution, wherein Xaa is any amino acid except proline.

In some aspects, the N-glycan sequon substitution corresponds to position 632-634 as compared to wild type H77 HCV or position 249-251 of SEQ ID NO:2. For example, disclosed are N-glycan sequon substitutions at position 632 and 634, based on the amino acid numbering of H77, that result in an asparagine at position 632 and a serine or threonine at position 634. In some aspects, the N-glycan sequon substitution corresponds to position 630-632. In some aspects, the N-glycan sequon substitution corresponds to position 628-630. In some aspects, the N-glycan sequon substitution corresponds to position 627-629.

In some aspects, the N-glycan sequon substitution is Y632N-G634S as compared to wild type H77. For example, a modified HCV E2 polypeptide comprising the N-glycan sequon substitution of Y249N-G251S compared to SEQ ID NO:2 comprises the sequence of ETHVTGGSAGRTTAGLVGLLTPGAKQNIQLINTNGSWHINSTALNCNESLNTGWLAG LFYQHKFNSSGCPERLASCRRLTDFAQGWGPISYANGSGLDERPYCWHYPPRPCGIV PAKSVCGPVYCFTPSPVVVGTTDRSGAPTYSWGANDTDVFVLNNTRPPLGNWFGCT WMNSTGFTKVCGAPPCVIGGVGNNTLLCPTDCFRKHPEATYSRCGSGPWITPRCMV DYPYRLWHYPCTINYTIFKVRMNVSGVEHRLEAACNWTRGERCDLEDRDRSELSPL LLSTTQWQVLPCSFTTLPALSTGLIHLHQNIVDVQYLYGVGSSIASWAI (SEQ ID NO:7). A Y249N-G251S substitutions are shown in bold. In some aspects, a modified HCV E2 polypeptide comprising the N-glycan sequon substitution of Y632N-G634S consists of SEQ ID NO:7.

In some aspects, the N-glycan sequon substitution is R630N-Y632T as compared to wild type H77 or R247N-Y249T as compared to SEQ ID NO:2. In some aspects, the N-glycan sequon substitution is K628N-R630S as compared to wild type H77 or K245N-R247S as compared to SEQ ID NO:2. In some aspects, the N-glycan sequon substitution is F627N-V629T as compared to wild type H77 or F244N-V246T as compared to SEQ ID NO:2.

In some aspects, the N-glycan sequon substitution is in the antigenic domain A of an amino acid sequence with 70, 75, 80, 85, 90, 95 or 99% identity to SEQ ID NO:7, wherein the antigenic domain A comprises the N-glycan sequon substitution of Y632N-G634S as compared to wild type H77. In other words, the modified HCV E2 glycoprotein comprises a sequence with 70, 75, 80, 85, 90, 95, or 99% identity to SEQ ID NO:7, wherein the N-glycan sequon substitution in the antigenic domain A comprises an N at position 632 and an S at position 634 wherein the numbers correspond to the numbering of H77. Thus, the reason for the less than 100% identity is due to an alteration in the sequence somewhere other than the Y632N-G634S mutations corresponding to positions 632 and 634 of wild type H77.

In some aspects, the N-glycan sequon substitution is in the antigenic domain A of an amino acid sequence with 70, 75, 80, 85, 90, 95 or 99% identity to SEQ ID NO:7, wherein the antigenic domain A comprises the N-glycan sequon substitution of R630N-Y632T as compared to wild type H77. In some aspects, the modified HCV E2 glycoprotein comprises a sequence with 70, 75, 80, 85, 90, 95, or 99% identity to SEQ ID NO:2, wherein the N-glycan sequon substitution in the antigenic domain A comprises the an N at position 630 and an T at position 632 wherein the numbers correspond to the numbering of H77.

In some aspects, the N-glycan sequon substitution is in the antigenic domain A of an amino acid sequence with 70, 75, 80, 85, 90, 95 or 99% identity to SEQ ID NO:2, wherein the antigenic domain A comprises the N-glycan sequon substitution of K628N-R630S as compared to wild type H77. In some aspects, the modified HCV E2 glycoprotein comprises a sequence with 70, 75, 80, 85, 90, 95, or 99% identity to SEQ ID NO:7, wherein the N-glycan sequon substitution in the antigenic domain A comprises the an N at position 628 and a S at position 630 wherein the numbers correspond to the numbering of H77.

In some aspects, the N-glycan sequon substitution is in the antigenic domain A of an amino acid sequence with 70, 75, 80, 85, 90, 95 or 99% identity to SEQ ID NO:2, wherein the antigenic domain A comprises the N-glycan sequon substitution of F627N-V629T as compared to wild type H77. In some aspects, the modified HCV E2 glycoprotein comprises a sequence with 70, 75, 80, 85, 90, 95, or 99% identity to SEQ ID NO:2, wherein the N-glycan sequon substitution in the antigenic domain A comprises the an N at position 627 and a T at position 629 wherein the numbers correspond to the numbering of H77.

In some aspects, the N-glycan sequon substitutions can be combined with any of the amino acid alterations in the antigenic D domain of E2 described herein. For example, in some aspects, disclosed are modified HCV E2 glycoproteins comprising a proline substitution at the amino acid corresponding to position 445 of wild type H77 and an arginine substitution and serine or threonine substitution at the amino acids corresponding to positions 632 and 634, respectively, of wild type H77.

ii. Scaffold

Disclosed herein are modified HCV E1E2 glycoproteins comprising a HCV E1 polypeptide; a first scaffold element; a modified HCV E2 polypeptide; and a second scaffold element, wherein the HCV E1 polypeptide does not comprise a transmembrane domain; wherein the modified HCV E2 polypeptide does not comprise a transmembrane domain, wherein the modified HCV E2 polypeptide comprises an antigenic domain D, and wherein the modified HCV E2 polypeptide comprises one or more amino acid alterations in the antigenic domain D. In some aspects, the first scaffold element and second scaffold element are capable of interacting with each other forming a scaffold. In some aspects, the scaffold, and thus the scaffold elements, can be necessary for E1E2 assembly.

In some aspects, the presence of a first scaffold element and a second scaffold element of a modified HCV E1E2 glycoprotein can be in any order. Thus, in some aspects, the first scaffold element can be located on the C-terminus of the HCV E1 polypeptide and the second scaffold element can be located on the C-terminus of the modified HCV E2 polypeptide. In other instances, the first scaffold element can be located on the C-terminus of the modified HCV E2 polypeptide and the second scaffold element can be located on the C-terminus of the HCV E1 polypeptide.

In some aspects, the first scaffold element of a modified HCV E1E2 glycoprotein can be a subsequence of c-Jun and the second scaffold element is a subsequence of c-Fos. In some aspects, the first scaffold element of a modified HCV E1E2 glycoprotein is a subsequence of c-Fos and the second scaffold element is a subsequence of c-Jun. Thus, the first and second scaffold elements of a modified HCV) E1E2 glycoprotein can be reversed in the location they are found on the E1E2 glycoprotein as long as they still retain the ability to interact with each other, thus forming a scaffold. For example, the first scaffold and second scaffold can be capable of forming a leucine zipper. In an aspect, c-Jun and c-fos can interact with each other to form a leucine zipper

In some aspects, the c-Jun subsequence is RIARLEEKVKTLKAQNSELASTANMLREQVAQLKQKVMNY (SEQ ID NO:8). In some aspects, the c-Fos subsequence is

(SEQ ID NO: 9)
LTDTLQAETDQLEDKKSALQTEIANLLKEKEKLEFILAAY.

In some aspects, one or both of the c-Jun and c-Fos sequences can have a linker. In some aspects, the linker can be PGG. For example, the subsequence of c-Jun can be PGGRIARLEEKVKTLKAQNSELASTANMLREQVAQLKQKVMNY (SEQ ID NO:10) and/or the subsequence of c-Fos can be

(SEQ ID NO: 11)
PGGLTDTLQAETDQLEDKKSALQTEIANLLKEKEKLEFILAAY.

In some aspects, a scaffold can be composed of a single scaffold element, such as a foldon-based scaffold. When the scaffold is a single scaffold element, the scaffold (and thus single scaffold element) can be located on either the HCV E1 or E2 polypeptide. Thus, in some aspects, a modified HCV E1E2 glycoprotein can comprise a HCV E1 polypeptide; a scaffold; and a modified HCV E2 polypeptide; wherein the HCV E1 polypeptide does not comprise a transmembrane domain; wherein the modified HCV E2 polypeptide does not comprise a transmembrane domain, wherein the modified HCV E2 polypeptide comprises an antigenic domain D, and wherein the modified HCV E2 polypeptide comprises one or more amino acid alterations in the antigenic domain D. In some aspects, the scaffold can be present on the HCV E1 polypeptide or the modified HCV E2 polypeptide. For example, the scaffold can be a foldon-based scaffold. Thus, in some aspects scaffold elements are present on each of the HCV E1 polypeptide and the modified HCV E2 polypeptide, which can result in a scaffold, and in some aspects the HCV E1E2 glycoprotein comprises a scaffold on only one of the HCV E1 polypeptide or the modified HCV E2 polypeptide.

In some aspects, the first scaffold of a modified HCV E1E2 glycoprotein can be a first coiled-coil domain and the second scaffold is a second coiled-coil domain. In such an arrangement, the interaction of the first and second coiled-coil domains can provide a scaffold for the HCV E1E2 glycoprotein. In some aspects, a first or second coiled-coil domain can comprise the sequence of AAEDLLELAHTILKTARNQLRTMEILRKER (SEQ ID NO:3). In some aspects, if a first or second coiled-coil domain comprises SEQ ID NO:3, then the opposite coiled-coil domain can comprise the sequence of ADERRKAKELLKEAEEIWKRINELAERETK (SEQ ID NO:4). In such an arrangement, if the first coiled-coil domain is SEQ ID NO:3 then the second coiled-coil domain can be SEQ ID NO:4 or if the first coiled-coil domain is SEQ ID NO:4 then the second coiled-coil domain can be SEQ ID NO:3.

In some aspects, the first scaffold element and second scaffold element are not transmembrane domains. Thus, the E1E2 assembly is not due to the location of HCV E1 polypeptide in a cell membrane close to HCV E2 polypeptide.

iii. Cleavage Site

In some aspects, the disclosed modified HCV E1E2 glycoproteins can further comprise a cleavage site. For example, disclosed are modified HCV E1E2 glycoproteins comprising a HCV E1 polypeptide, wherein the HCV E1 polypeptide does not comprise a transmembrane domain; a first scaffold element; a cleavage site, a modified HCV E2 polypeptide, wherein the HCV E2 polypeptide does not comprise a transmembrane domain, wherein the modified HCV E2 polypeptide comprises an antigenic domain D, and wherein the modified HCV E2 polypeptide comprises one or more amino acid alterations in the antigenic domain D; and a second scaffold element.

In some aspects, the cleavage site is located between the HCV E1 polypeptide and the modified HCV E2 polypeptide. In some aspects, the cleavage site can be located after the first scaffold element and before the modified HCV E2 polypeptide.

In some aspects, the cleavage site can be a furin cleavage site. In some aspects, the furin cleavage site comprises six arginines (RRRRRR; SEQ ID NO: 12). In some aspects, the furin cleavage site can be RRRRKR (SEQ ID NO:13) or RRRKKR (SEQ ID NO:14). In some aspects, the furin cleavage site is R-X-K/R-R (SEQ ID NO:15/16). In some aspects, the furin cleavage site can be, but is not limited to Tobacco Etch Virus (TEV) protease cleavage site (ENLYFQS; SEQ ID NO:17) or human rhinovirus type 14 (HRV) 3C protease cleavage site (LEVLFQGP; SEQ ID NO:18).

In some aspects, the cleavage site can be present when the modified HCV E1E2 glycoprotein is expressed as a single polypeptide. The cleavage site can then be used to cleave the HCV E1 polypeptide from the modified HCV E2 polypeptide which would allow the HCV E1 polypeptide and the modified HCV E2 polypeptide to come together via the scaffold (e.g. first scaffold element and second scaffold element) correctly assembling the E1E2 glycoprotein.

In some aspects, the disclosed modified HCV E1E2 glycoproteins do not comprise a cleavage site. For example, in some aspects, if the HCV E1 polypeptide and the modified HCV E2 polypeptide of a modified HCV E1E2 glycoprotein are co-expressed in trans, then a cleavage site may not be necessary.

iv. Leader Sequence

In some aspects, the disclosed modified HCV E1E2 glycoproteins can further comprise a leader sequence at the N-terminal end of the HCV E1 polypeptide. For example, disclosed are modified HCV E1E2 glycoproteins comprising a HCV E1 polypeptide, wherein the HCV E1 polypeptide does not comprise a transmembrane domain, wherein the HCV E1 polypeptide comprises a leader sequence at the N-terminal end of the HCV E1 polypeptide; a first scaffold element; a HCV E2 polypeptide, wherein the HCV E2 polypeptide does not comprise a transmembrane domain, wherein the modified HCV E2 polypeptide comprises an antigenic domain D, and wherein the modified HCV E2 polypeptide comprises one or more amino acid alterations in the antigenic domain D; and a second scaffold element.

In some aspects, the leader sequence can be a tissue plasminogen activator (tPA) leader sequence. In some aspects, the leader sequence can be derived from human IL-2 or murine Ig-kappa. In some aspects, the leader sequence can be any of the leader sequences provided in Table 3.

v. Other Moieties

In some aspects, additional sequences that aid in solubilizing, detecting, and/or purifying the HCV E1E2 glycoprotein can be added to one or more elements of the disclosed glycoproteins. In some aspects, the disclosed modified HCV E1E2 glycoproteins can further comprise a detectable label or diagnostic moiety.

In some aspects, the disclosed modified HCV E1E2 glycoproteins can further comprise a detectable moiety. In some aspects, the detectable moiety can be located at the C-terminal end of the second scaffold element. For example, disclosed are modified HCV E1E2 glycoproteins comprising a HCV E1 polypeptide, wherein the HCV E1 polypeptide does not comprise a transmembrane domain; a first scaffold element; a HCV E2 polypeptide, wherein the HCV E2 polypeptide does not comprise a transmembrane domain, wherein the modified HCV E2 polypeptide comprises an antigenic domain D, and wherein the modified HCV E2 polypeptide comprises one or more amino acid alterations in the antigenic domain D; and a second scaffold element, wherein the second scaffold element comprises a detectable moiety.

In some aspects, the detectable moiety is a purification tag or a label. As used herein, a detectable moiety, is any molecule that can be associated with a HCV E1 polypeptide, HCV E2 polypeptide, a first scaffold element, or a second scaffold element, directly or indirectly, and which results in a measurable, detectable signal, either directly or indirectly. Many such detectable moieties are known to those of skill in the art. Examples of detectable moieties can be, but are not limited to, radioactive isotopes, fluorescent molecules, phosphorescent molecules, enzymes, antibodies, and ligands.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horseradish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like. Other labels can include biotin, streptavidin, horseradish peroxidase, or luciferase.

In some aspects, the disclosed modified HCV E1E2 glycoproteins can further comprise a maltose binding protein sequence. A maltose binding protein can help with protein solubilization, protein detection, and protein purification. In some aspects, the disclosed modified HCV E1E2 glycoproteins can further comprise a histidine tag. A histidine tag can be used for protein purification and detection. Those of skill in the art would understand those known sequences available for solubilizing, detecting, and/or purifying polypeptides that can be used with the disclosed modified HCV E1E2 glycoproteins.

In an exemplary embodiment, carrier proteins represented by virus capsid proteins that have the capability to self-assemble into virus-like particles (VLPs) are utilized in combination with the disclosed modified HCV E1E2 glycoproteins. Examples of VLPs used as peptide carriers are hepatitis B virus surface antigen and core antigen, hepatitis E virus particles, polyoma virus, bovine papilloma virus, and the like.

In another embodiment, the disclosed modified HCV E1E2 glycoproteins can be coupled to one of a number of carrier molecules, known to those of skill in the art. A carrier protein should be of sufficient size for the immune system of the subject to which it is administered to recognize its foreign nature and develop antibodies to it.

In some cases a carrier molecule can be directly coupled to the disclosed modified HCV E1E2 glycoproteins. In other cases, there is a linker molecule inserted between the carrier molecule and the disclosed modified HCV E1E2 glycoproteins. For example, the coupling reaction may require a free sulfhydryl group on the peptide. In such cases, an N-terminal cysteine residue is added to the modified HCV E1E2 glycoprotein when the modified HCV E1E2 glycoprotein is synthesized. In an exemplary embodiment, traditional succinimide chemistry is used to link the modified HCV E1E2 glycoprotein to a carrier protein. Methods for preparing such modified HCV E1E2 glycoprotein:carrier protein conjugates are generally known to those of skill in the art and reagents for such methods are commercially available (e.g., from Sigma Chemical Co.). Generally about 5-30 modified HCV E1E2 glycoprotein molecules are conjugated per molecule of carrier protein.

Any of the disclosed modified HCV E1E2 glycoproteins can be combined with other viral subunits to form an attenuated live virus or replication-defective virus. In some aspects, the disclosed modified HCV E1E2 glycoproteins can be combined with other elements to form nanoparticles carrying the disclosed modified HCV E1E2 glycoproteins.

In some aspects, the disclosed modified HCV E1E2 glycoproteins can be combined with one or more of the modifications to HCV E2 polypeptide as described in U.S. Pat. No. 9,732,121, which is hereby incorporated by reference in its entirety for its teaching of modifications to HCV E2 polypeptide.

### C. Nucleic Acid Sequences

Disclosed are polynucleotides comprising a nucleic acid sequence capable of encoding one or more of the disclosed modified HCV glycoproteins.

### D. Vectors

Disclosed are vectors comprising any of the polynucleotides disclosed herein.

The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the present disclosure is intended to include other vectors which serve equivalent functions.

In some aspects, the vector can be a viral vector. For example, the viral vector can be an adeno-associated viral vector. In some aspects, the vector can be a non-viral vector, such as a DNA based vector.

1. Viral and Non-Viral Vectors

There are a number of compositions and methods which can be used to deliver the disclosed nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence capable of encoding a VMD2 promoter operably linked to a nucleic acid sequence encoding Rap 1a.

The “control elements” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. If it is necessary to generate a cell line that contains multiple copies of the sequence encoding a polypeptide, vectors based on SV40 or EBV may be advantageously used with an appropriate selectable marker.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the present disclosure. In certain constructs the promoter or enhancer region can be active in all eukaryotic cell types, even if it is only expressed in a particular type of cell at a particular time.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product can be used to determine if the gene has been delivered to the cell and once delivered is being expressed. Marker genes can include, but are not limited to the E. coli lacZ gene, which encodes ß-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

Another type of selection that can be used with the composition and methods disclosed herein is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puromycin.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Moloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package, which has packed into it, nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the present disclosure into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subject's lung cells. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

### E. Cells and Cell Lines

Disclosed herein are cells and cell lines comprising the disclosed modified HCV E1E2 glycoproteins, nucleic acid sequences, vectors or compositions disclosed herein.

As used herein, the terms “cell,” “cell line,” and “cell culture” can be used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

Suitable host cells for cloning or expressing the DNA or harboring the disclosed modified HCV E1E2 glycoproteins are the prokaryote, yeast, or higher eukaryote cells. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR(CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1.982)); MRC 5 cells; FS4 cells; and a human hepatoma line (Hep G2).

Host cells are transformed with the above-described expression or cloning vectors for modified HCV E1E2 glycoprotein production and cultured in conventional nutrient media modified as appropriate for inducing promoters, selecting transformants, or amplifying the genes encoding the desired sequences.

The disclosed modified HCV E1E2 glycoprotein compositions can be prepared from the cells can be purified using, for example, hydroxylapatite chromatography, gel electrophoresis, dialysis, and affinity chromatography, and the like as known in the art. For example, antibodies against E2 protein can be used as affinity reagents for purification. The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

### F. Compositions

Disclosed are compositions comprising one or more of the modified HCV E1E2 glycoproteins described herein and a pharmaceutically acceptable carrier thereof.

In some aspects, the composition can be a pharmaceutical composition (e.g., formulation, preparation, medicament) comprising, or consisting essentially of, or consisting of as an active ingredient, a modified HCV E2 glycoprotein, modified membrane bound HCV E1E2 glycoprotein, a nucleic acid construct, vector, or protein as described herein, and a pharmaceutically acceptable carrier, diluent, or excipient.

Disclosed are compositions and formulations of the disclosed modified HCV EE2 glycoproteins with a pharmaceutically acceptable carrier or diluent. For example, disclosed are pharmaceutical compositions, comprising a HCV E1E2 glycoprotein comprising a HCV E1 polypeptide; a first scaffold element; a HCV E2 polypeptide; and a second scaffold element, wherein the HCV E1 polypeptide does not comprise a transmembrane domain, and wherein the HCV E2 polypeptide does not comprise a transmembrane domain, and a pharmaceutically acceptable carrier.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this disclosure. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the present disclosure is not compromised. Pharmaceutical compositions may also include one or more active ingredients (in addition to the composition of the present disclosure) such as antimicrobial agents, anti-inflammatory agents, anesthetics, and the like. In the methods described herein, delivery of the disclosed compositions to cells can be via a variety of mechanisms. The pharmaceutical composition may be administered in a number of ways depending on whether local or systemic treatment is desired, and on the area to be treated.

In some aspects, the disclosed compositions can be a vaccine. A vaccine is a pharmaceutical composition that is safe to administer to a subject animal, and is able to induce protective immunity in that animal against a pathogenic micro-organism, i.e. to induce a successful protection against an infection with the micro-organism. In some aspects, protection against an infection with a micro-organism is aiding in preventing, ameliorating or curing an infection with that micro-organism or a disorder arising from that infection, for example to prevent or reduce one or more clinical signs associated with the infection with the pathogen.

By the term “vaccine” as used herein, is meant a composition; a formulation comprising a composition disclosed herein; a virus or virus-like particle comprising a modified HCV E1E2 glycoprotein of the present disclosure; or a nucleic acid sequence encoding a modified HCV E1E2 glycoprotein disclosed herein, which, when administered to a subject, induces cellular or humoral immune responses as described herein.

Some embodiments and compositions described herein provide a method of stimulating an immune response in a mammal, which can be a human or a preclinical model for human disease, e.g. mouse, ape, monkey etc. “Stimulating an immune response” includes, but is not limited to, inducing a therapeutic or prophylactic effect that is mediated by the immune system of the mammal. More specifically, stimulating an immune response in the context of the present disclosure refers to eliciting cellular or humoral immune responses, thereby inducing downstream effects such as production of antibodies, antibody heavy chain class switching, maturation of APCs, and stimulation of cytolytic T cells, T helper cells and both T and B memory cells.

As appreciated by skilled artisans, vaccine compositions are suitably formulated to be compatible with the intended route of administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH of the composition can be adjusted with acids or bases, such as hydrochloric acid or sodium hydroxide. Systemic administration of the composition is also suitably accomplished by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

Vaccine compositions may include an aqueous medium, pharmaceutically acceptable inert excipient such as lactose, starch, calcium carbonate, and sodium citrate. Vaccine compositions may also include an adjuvant, for example Freud's adjuvant. Vaccines may be administered alone or in combination with a physiologically acceptable vehicle that is suitable for administration to humans. Vaccines may be delivered orally, parenterally, intramuscularly, intranasally or intravenously. Oral delivery may encompass, for example, adding the compositions to the feed or drink of the mammals. Factors bearing on the vaccine dosage include, for example, the weight and age of the mammal. Compositions for parenteral or intravenous delivery may also include emulsifying or suspending agents or diluents to control the delivery and dose amount of the vaccine.

The modified HCV E1E2 glycoprotein and polynucleotides that encode such modified HCV E1E2 glycoprotein can be used in various HCV vaccine formulations known in the art, as a substitution for a wild-type HCV E1E2 sequence.

In some aspects, disclosed are vaccines comprising HCV E1E2 glycoproteins comprising a HCV E1 polypeptide; a first scaffold element; a HCV E2 polypeptide; and a second scaffold element, wherein the HCV E1 polypeptide does not comprise a transmembrane domain, and wherein the HCV E2 polypeptide does not comprise a transmembrane domain.

In some aspects, disclosed are vaccines comprising HCV E1E2 glycoproteins comprising a HCV E1 polypeptide; a first scaffold element; a modified HCV E2 polypeptide; and a second scaffold element, wherein the HCV E1 polypeptide does not comprise a transmembrane domain, and wherein the modified HCV E2 polypeptide does not comprise a transmembrane domain, wherein the modified HCV E2 polypeptide comprises an antigenic domain D, wherein the modified HCV E2 polypeptide comprises one or more amino acid alterations in the antigenic domain D. In some aspects, at least one amino acid alteration is a proline substitution as disclosed herein.

The disclosed modified HCV E1E2 glycoproteins and nucleic acid sequences that encode such modified HCV E1E2 glycoproteins can be used in various HCV vaccine formulations known in the art, as a substitution for the wild-type HCV E1E2 sequence. In some aspects, the disclosed vaccines are live-attenuated virus, replication-defective viruses, nanoparticles, or subunit vaccines wherein each of them comprise one of the disclosed modified HCV E1E2 glycoproteins. In some aspects, the modified HCV E1E2 glycoproteins can help form a live-attenuated virus or replication-defective virus vaccine. In some aspects, the disclosed vaccines can be mRNA vaccines comprising one of the disclosed nucleic acid sequences. For example, the disclosed vaccines can be mRNA vaccines comprising a nucleic acid sequence that encodes one of the disclosed modified HCV E1E2 glycoproteins.

1. Delivery of Compositions

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

### G. Methods

Disclosed are methods of increasing HCV E1E2 glycoprotein immunogenicity in a subject in need thereof comprising administering a composition comprising one or more of the modified HCV E1E2 glycoproteins. In some aspects, serum from the subject comprises anti-EE2 antibodies at least 2 weeks after administration. In some aspects, serum from the subject comprises anti-E1E2 antibodies at least 2, 4, 6, 8, 10, or 12 weeks after administration. In some aspects, serum from the subject comprises anti-E1E2 antibodies at least 1 week, 1 month, or 1 year after administration. In some aspects, the anti-EE2 antibodies are neutralizing antibodies. Thus, the disclosed are methods of increasing HCV E1E2 glycoprotein immunogenicity in a subject can result in an increase of neutralizing antibodies in the subject.

Disclosed are methods of increasing HCV E1E2 glycoprotein antigenicity in a subject in need thereof comprising administering a composition comprising one or more of the modified HCV E1E2 glycoproteins described herein. In some aspects, the modified HCV E1E2 glycoproteins having an alteration in the HCV E2 polypeptide antigenic domain D described herein can be administered, wherein the increase in HCV E1E2 glycoprotein antigenicity is an increase in the HCV E2 polypeptide antigenic domain D antigenicity. In some aspects, disclosed are methods of increasing HCV E1E2 glycoprotein antigenicity in a subject in need thereof comprising administering a composition comprising one or more of the modified HCV E1E2 glycoproteins disclosed herein, wherein the modified HCV E2 polypeptide comprises an antigenic domain D, wherein the modified HCV E2 polypeptide comprises one or more amino acid alterations in the antigenic domain D, wherein at least one amino acid alteration is a proline substitution, and wherein the increase in HCV E1E2 glycoprotein antigenicity is an increase in HCV E2 polypeptide antigenic domain D antigenicity. For example, a proline substitutions can be a proline substitution as disclosed herein, such as the H62P substitution found in SEQ ID NO:6 and can increase the antigenicity of HCV E2 polypeptide. In some aspects, the presence of a proline substitution in the antigenic domain D near an antibody binding site can help stabilize the epitope resulting in increased antigenicity. In some aspects, the modified HCV E2 glycoprotein can further comprise an N-glycan sequon in the antigenic domain A. In some aspects, the modified HCV E2 glycoprotein can further comprise an N-glycan sequon in the antigenic domain A wherein the antigenicity of antigenic domain A is masked and the antigenicity in antigenic domain D is increased.

Disclosed are method of decreasing HCV E1E2 glycoprotein antigenicity in a subject in need thereof comprising administering a composition comprising one or more of the modified HCV E1E2 glycoproteins having an alteration in the HCV E2 polypeptide antigenic domain A described herein, wherein the decrease in HCV E1E2 glycoprotein antigenicity is a decrease in HCV E2 polypeptide antigenic domain A antigenicity. In some aspects, disclosed are methods of decreasing HCV E1E2 glycoprotein antigenicity in a subject in need thereof comprising administering a composition comprising one or more of the modified HCV E1E2 glycoproteins comprising a modified HCV E2 polypeptide comprising an antigenic domain A, wherein the antigenic domain A comprises an N-glycan sequon substitution, wherein the decrease in HCV E1E2 glycoprotein antigenicity is a decrease in antigenic domain A antigenicity of the HCV E2 polypeptide. In some aspects, the N-glycan sequeon substitution in the antigenic domain A masks an epitope, therefore decreasing the antigenicity of antigenic domain A. In some aspects, the antigenic domain A is known to be associated with non-neutralizing antibodies. In some aspects, by masking this region and diverting the antibody response to other regions, such as the antigenic domain D, that neutralizing antibodies can bind can be a good mechanism for vaccine development. In some aspects, any of the modified HCV E1E2 glycoproteins comprising the N-glycan sequon substitution in the antigenic domain A of the modified HCV E2 polypeptide can be used in these methods.

Disclosed are methods of inducing an immune response in a subject in need thereof comprising administering to the subject in need thereof a composition comprising one or more of the modified HCV E1E2 glycoproteins disclosed herein. Disclosed are methods of inducing an immune response in a subject in need thereof comprising administering to the subject in need thereof a composition comprising one or more of the modified HCV E1E2 glycoproteins comprising comprising a HCV E1 polypeptide; a first scaffold element; a HCV E2 polypeptide; and a second scaffold element, wherein the HCV E1 polypeptide does not comprise a transmembrane domain, and wherein the HCV E2 polypeptide does not comprise a transmembrane domain. Disclosed are methods of inducing an immune response in a subject in need thereof comprising administering to the subject in need thereof a composition comprising one or more of the modified HCV E1E2 glycoproteins comprising comprising a HCV E1 polypeptide; a first scaffold element; a modified HCV E2 polypeptide; and a second scaffold element, wherein the HCV E1 polypeptide does not comprise a transmembrane domain, and wherein the HCV E2 polypeptide does not comprise a transmembrane domain, wherein the modified HCV E2 polypeptide comprises an antigenic domain D, wherein the modified HCV E2 polypeptides comprise one or more amino acid alterations in the antigenic domain D, wherein at least one amino acid alteration is a proline substitution. In some aspects of the disclosed methods of inducing an immune response in a subject in need thereof, the immune response is an antibody response wherein the antibodies can bind to HCV. In some aspects, the modified HCV E2 polypeptide comprising an antigenic domain D, wherein the modified HCV E2 polypeptide comprises one or more amino acid alterations in the antigenic domain D, wherein at least one amino acid alteration is a proline substitution induces a stronger or more potent antibody response than an HCV E1E2 glycoprotein not having a proline substitution in the antigenic domain D of the E2 polypeptide. For example, the disclosed modified HCV E1E2 glycoproteins, specifically the ones with the modified HCV E2 polypeptide comprising an antigenic domain D, wherein the modified HCV E2 glycoproteins comprise one or more amino acid alterations in the antigenic domain D, wherein at least one amino acid alteration is a proline substitution, induce a stronger or more potent antibody response than the wild type H77 E2 glycoprotein.

In some aspects of any of the disclosed methods herein, the subject in need thereof has been infected with HCV or is at risk for being infected with HCV.

Also disclosed are methods of treating a subject having HCV or at risk of being infected with HCV comprising administering to the subject a composition comprising one or more of the modified HCV E1E2 glycoproteins disclosed herein. In some aspects, treating a subject can include preventing further infection in a subject already infected with HCV. In some aspects, treating a subject can include preventing infection or viral replication in a subject exposed to HCV. In some aspects, the modified HCV E1E2 glycoprotein induces an immune response against HCV in the subjects. In some aspects, the modified HCV E1E2 glycoproteins can be any of the modified HCV E1E2 glycoproteins comprising a modified HCV E2 polypeptide comprising a proline substitution in the antigenic domain D and/or an N-glycan sequon substitution in antigenic domain A.

Disclosed are methods of generating neutralizing antibodies (nAbs) a subject in need thereof comprising administering to the subject in need thereof a composition comprising one or more of the modified HCV E1E2 glycoproteins disclosed herein. In some aspects, the nAbs inhibit HCV infection in the subject. In some aspects, the nAbs inhibit HCV infection from all HCV genotypes, specifically genotypes 1 through 7. In some aspects, the nAbs are directed to the antigenic domain D of HCV E2 polypeptide. In some aspects, the subject in need thereof has been infected with HCV or is at risk for being infected with HCV. In some aspects, the modified HCV E2 glycoproteins can be any of the modified HCV E2 glycoproteins comprising a proline substitution in the antigenic domain D. In some aspects, the modified HCV E2 polypeptides comprise a proline substitution in the antigenic domain D and an N-glycan sequon substitution in antigenic domain A.

Also disclosed are methods for immunizing a subject in need thereof comprising administering to the subject in need thereof a composition comprising one or more of the modified HCV E1E2 glycoproteins disclosed herein. In some aspects, the subject in need thereof has been infected with HCV or is at risk for being infected with HCV. In some aspects, the modified HCV E1E2 glycoproteins can be any of the modified HCV E1E2 glycoproteins, including those comprising a modified HCV E2 polypeptide comprising a proline substitution in the antigenic domain D. In some aspects, the modified HCV E2 polypeptides comprising a proline substitution in the antigenic domain D further comprise an N-glycan sequon substitution in antigenic domain A. In some aspects, a protective immune response effective to reduce or eliminate subsequent HCV infection clinical signs in the subject, relative to a non-immunized control subject of the same species, is elicited by administration of the composition. In some aspects, a protective immune response effective to reduce risk of HCV infection in the subject, relative to a non-immunized control subject of the same species, is elicited by administration of the composition.

In the methods disclosed herein, an immunologically effective amount of one or more disclosed modified HCV E1E2 glycoproteins, which may be conjugated to a suitable carrier molecule, polynucleotides encoding such modified polypeptides, including viral vectors, can be administered to a subject by administrations of a vaccine, in a manner effective to result in an improvement in the subject's condition.

In some aspects of any of the disclosed methods, the composition can be administered in a therapeutically effective amount. By an “effective amount” of a composition as provided herein is meant a sufficient amount of the composition to provide the desired effect. The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “effective amount.” However, an appropriate “effective amount” may be determined by one of skill in the art using only routine experimentation. The term “therapeutically effective amount” means an amount of a therapeutic, prophylactic, and/or diagnostic agent (e.g., modified HCV E1E2 glycoprotein) that is sufficient, when administered to a subject suffering from or susceptible to infection with HCV, to treat, alleviate, ameliorate, relieve, alleviate symptoms of, prevent, delay onset of, inhibit progression of, reduce severity of, and/or reduce incidence of infection with HCV. The term “immunologically effective amount” means an amount of a therapeutic, prophylactic, and/or diagnostic agent (e.g., modified HCV E1E2 glycoproteins) that is sufficient, when administered to a subject suffering from or susceptible to infection with HCV, to treat, alleviate, ameliorate, relieve, alleviate symptoms of, prevent, delay onset of, inhibit progression of, reduce severity of, and/or reduce incidence of infection with HCV based on an immune response.

In some aspects, the modified glycoproteins are used in a screening method to select for antibodies optimized for affinity, specificity, and the like. In such screening methods, random or directed mutagenesis is utilized to generate changes in the amino acid structure of the variable region or regions, where such variable regions will initially comprise one or more of the provided CDR sequences, e.g. a framework variable region comprising CDR1, CDR2, CDR3 from the heavy and light chain sequences. Methods for selection of antibodies with optimized specificity, affinity, etc., are known and practiced in the art, e.g. including methods described by Presta (2006) Adv Drug Deliv Rev. 58(5-6):640-56; Levin and Weiss (2006) Mol Biosyst. 2(1):49-57; Rothe et al. (2006) Expert Opin Biol Ther. 6(2):177-87; Ladner et al. (2001) Curr Opin Biotechnol. 12(4):406-10; Amstutz et al. (2001) Curt Opin Biotechnol. 12(4):400-5; Nakamura and Takeo (1998) J Chromatogr B Biomed Sci Appl. 715(1):125-36 each herein specifically incorporated by reference for teaching methods of mutagenesis selection. Such methods are exemplified by Wu et al. (2005) J. Mol. Biol. (2005) 350, 126-144.

In some aspects of the disclosed methods, the composition can be administered subcutaneously, intramuscularly, intravenously, intradermally, or orally.

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example, disclosed are kits comprising one or more of the disclosed modified HCV E1E2 glycoproteins, nucleic acids, vectors, or compositions.

### EXAMPLES

### A. Induction of Broadly Neutralizing Antibodies Using a Secreted Form of the Hepatitis C Virus E1E2 Heterodimer as a Vaccine

1. Introduction

Hepatitis C virus (HCV) is a global disease burden, with an estimated 71 million people infected worldwide (WHO (2017) (World Health Organization, Geneva)), (Waheed et al., World J Gastroenterol 24, 4959-4961 (2018)). Roughly 75% of HCV infections become chronic (Moosavy et al., Electron Physician 9, 5646-5656 (2017)). (Zaltron et al., BMC Infect Dis 12 Suppl 2, S2 (2012)), (Ansaldi et al., World J Gastroenterol 20, 9633-9652 (2014)), and in severe cases can result in cirrhosis or hepatocellular carcinoma (Buhler et al., Dig Dis 30, 445-452 (2012)). Viral infection can be cured at high rates by direct acting antivirals (DAAs), but several issues have blunted their effectiveness in eradicating HCV. In particular, multiple public health and financial barriers (Bartenschlager et al., Virus Res 248, 53-62 (2018)), (Al-Khazraji et al., Dig Dis 38, 46-52 (2020)) restrict access to DAAs in areas with high incidence of infection and DAAs do not prevent reinfection. Moreover, HCV infection is largely asymptomatic and often does not generate sterilizing immunity, thereby contributing to reinfection or continued disease progression (Bartenschlager et al., Virus Res 248, 53-62 (2018)), (Roche, et al., Liver Int 38 Suppl 1, 139-145 (2018)), (Midgard et al., J Hepatol 64, 1020-1026 (2016)). Collectively, these issues have resulted in a continued rise in HCV infections.

Acute HCV infections can be cleared by host immunity in approximately 25% of cases. Among individuals who clear their first infection, the rate of clearance rises to 80% for subsequent infections, indicating an effective immune memory response (Mehta et al., Lancet 359, 1478-1483 (2002)), (Page et al., J Infect Dis 200, 1216-1226 (2009)), (Osburn et al., Gastroenterology 138, 315-324 (2010)), (Bowen et al., Nature 436, 946-952 (2005)). This type of natural protective immunity to HCV requires the induction of broadly neutralizing antibodies to E1E2 ectodomains and T cell responses to the structural and non-structural proteins (Walker, Cold Spring Harbor perspectives in medicine 9 (2019)), (Holz et al., Antiviral Res 114, 96-105 (2015)), (Bailey et al., Gastroenterology 156, 418-430 (2019)). The above clinical observations indicate that, if a vaccine candidate could induce broadly neutralizing antibody and cell-mediated immune responses equivalent to that seen in spontaneous clearance, such a vaccine would be highly effective at preventing HCV infection. An HCV vaccine therefore remains an essential proactive measure to protect against viral spread, yet vaccine developments against the virus have been unsuccessful to date (Bailey et al., Gastroenterology 156, 418-430 (2019)), (Duncan et al., Vaccines (Basel) 8 (2020)). A number of challenges exist that have thus far limited progress towards developing a prophylactic vaccine against HCV. One major challenge in developing a successful vaccine for HCV has been the remarkable genetic diversity of the virus which has six major genotypes (genotypes 1-6), in addition to two less common genotypes (Borgia et al., The Journal of infectious diseases 218, 1722-1729 (2018)) (genotypes 7-8), and intra-genotypic diversity resulting in 90 total subtypes. Moreover, shielding of important neutralizing epitopes with glycans (Lavie, et al., Front Immunol 9, 910 (2018)), (Helle et al., J Virol 84, 11905-11915 (2010)), and the presence of immunodominant non-neutralizing epitopes (Brasher et al., Journal of hepatology 72, 670-679 (2020)), (Cashman et al., Front Immunol 5, 550 (2014)), (Pierce et al., Current opinion in virology 20, 55-63 (2016)), (Prentoe et al., Front Immunol 9, 2146 (2018)) deflect the immune response from conserved regions that mediate virus neutralization. Multiple studies in chimpanzees and humans have used E1E2 formulations to induce a humoral immune response, but their success in generating high titers of broadly neutralizing antibody (bnAb) responses has been limited. In particular, immunological assessment in chimpanzees of an E1E2 vaccine produced superior immune responses as compared to E2 administered alone and resulted in sterilizing immunity against homologous virus challenge (Choo et al., Proc Natl Acad Sci USA 91, 1294-1298 (1994)), (Houghton, Immunol Rev 239, 99-108 (2011)), but with less cross-neutralization capacity against heterologous isolates (Meunier et al., The Journal of infectious diseases 204, 1186-1190 (2011)). In addition, an E1E2 formulation tested in humans is well-tolerated (Frey et al., Vaccine 28, 6367-6373 (2010)). However, due to the limited neutralization breadth observed in the human clinical trial (Law et al., PLoS One 8, e59776 (2013)), (Stamataki, et al., J Infect Dis 204, 811-813 (2011)), using native E1E2 as a vaccine is not likely to provide sufficient protection from HCV infection. Rather, optimization of E1E2 to improve its immunogenicity and capacity to elicit bnAbs through rational design appears to be the preferred path for developing an effective B cell based vaccine (Kong, et al., Current opinion in virology 11, 148-157 (2015)).

An additional bottleneck contributing to the difficulty in generating protective B cell immune responses required for an effective HCV vaccine is preparation of a homogeneous E1E2 antigen. HCV envelope glycoproteins E1 and E2 form a heterodimer on the surface of the virion (Penin, et al., Hepatology 39, 5-19 (2004)), (Lapa, et al., Cells 8 (2019)), (Lavie, et al., Curr Issues Mol Biol 9, 71-86 (2007)). Furthermore, E1E2 assembly has been proposed to form a trimer of heterodimers (Falson et al., J Virol 89, 10333-10346 (2015)) mediated by hydrophobic C-terminal transmembrane domains (TMDs) (Lavie et al., Curr Issues Mol Biol 9, 71-86 (2007)), (Cocquerel, et al., J Virol 74, 3623-3633 (2000)), (De Beeck et al., J Biol Chem 275, 31428-31437 (2000)) and interactions between E1 and E2 ectodomains, (Bianchi et al., Int J Hepatol 2011, 968161 (2011)), (Haddad et al., J Virol 91 (2017)), (Vieyres, et al., Viruses 6, 1149-1187 (2014)). These glycoproteins are necessary for viral entry and infection, as E2 attaches to the CD81 and scavenger receptor type B class I (SR-B1) co-receptors as part of a multi-step entry process on the surface of hepatocytes (Colpitts, et al., Int J Mol Sci 21 (2020)), (Zeisel, et al., Curr Top Microbiol Immunol 369, 87-112 (2013)), (Pileri et al., Science 282, 938-941 (1998)), (Scarselli et al., The EMBO journal 21, 5017-5025 (2002)). Neutralizing antibody responses to HCV infection target epitopes in E1, E2, or the E1E2 heterodimer (Pierce, et al., Current opinion in virology 20, 55-63 (2016)), (Kinchen et al., J Clin Invest 130, 4786-4796 (2019)), (Tzarum, et al., Front Immunol 9, 1315 (2018)), (Wang, et al., Viruses 3, 2127-2145 (2011)), (Colbert et al., J Virol 93 (2019)), (Flyak et al., Cell Host Microbe 24, 703-716 e703 (2018)), (Keck et al., PLoS Pathog 15, e1007772 (2019)). A significant impediment to the uniform production of an immunogenic E1E2 heterodimer that could be utilized for vaccine development is the association of the antigen with the membrane via the TMDs (Lavie, et al., Curr Issues Mol Biol 9, 71-86 (2007)), (Zazrin, et al., Biochim Biophys Acta 1838, 784-792 (2014)). Progress has been made in the production and purification of the membrane-bound E1E2 complex via immunoaffinity purification (Lambot et al., J Biol Chem 277, 20625-20630 (2002)), (Pierce et al., J Virol 94 (2020)) or the use of tags that allow protein A (Logan et al., J Virol 91 (2017)) or anti-Flag (Krapchev et al., Virology 519, 33-41 (2018)) chromatography. While these methods produce high quality samples, they all involve harsh elution conditions. How such conditions might influence sample quality at a scale required for vaccine trials is unclear. Further, intracellular expression and membrane extraction limits the ability to produce large quantities of sufficient homogeneity required for both basic research and vaccine production. In contrast, viral glycoproteins of influenza hemagglutinin (Lu et al., Proc Natl Acad Sci USA 111, 125-130 (2014)), respiratory syncytial virus (RSV) (McLellan et al., Science 342, 592-598 (2013)), SARS-CoV-2 (Kim et al., EBioMedicine, 102743 (2020)), and others (Tai et al., Virology 499, 375-382 (2016)), (Chang et al., Appl Microbiol Biotechnol 102, 7499-7507 (2018)) have been stabilized in soluble form using a C-terminal attached foldon trimerization domain to facilitate assembly. In addition, HIV gp120-gp41 proteins have been designed as soluble SOSIP trimers in part by introducing a furin cleavage site to facilitate native-like assembly when cleaved by the enzyme (Sanders et al., PLoS Pathog 9, e1003618 (2013)), (Leblanc et al., Hum Vaccin Immunother 10, 3022-3038 (2014)). Recent efforts have made strides toward liberating the E1E2 complex from the membrane in its native form (Cao et al., PLoS Pathog 15, e1007759 (2019)), (Guest et al., Proc Natl Acad Sci U.S.A. 118 (2021)). In particular, previous work (Guest et al., Proc Natl Acad Sci USA 118 (2021)) showed that a soluble E1E2 (sE1E2) using the Fos/Jun leucine zipper coiled coil as a scaffold (sE1E2.LZ) is antigenically intact, as the protein is recognized by E1E2-specific mAbs AR4A and AR5A (Giang et al., Proc Natl Acad Sci USA 109, 6205-6210 (2012)). Moreover, sE1E2.LZ elicited neutralizing antibodies in mice immunized with the antigen, making this scaffold a promising potential platform for engineering of additional HCV vaccine candidates.

Here, the immunogenicity of the native-like secreted E1E2 construct sE1E2.LZ is described and compare it to the membrane-bound E1E2 complex (mbE1E2) and a secreted form of the E2 ectodomain (sE2). Immunization of mice with sE1E2.LZ produced sera possessing anti-E1E2 antibodies at levels comparable to mice immunized with mbE1E2 or sE2. Moreover, the antibody response in sE1E2.LZ-immunized mice is skewed more towards neutralizing antibodies relative to non-neutralizing antibodies than the other two antigens. Remarkably, sera from sE1E2.LZ-immunized mice exhibited broader neutralization activity than either mbE1E2 or sE2 when assessed using both pseudotyped HCV particles (HCVpp) and cell culture-derived HCV (HCVcc), indicating that this sE1E2 platform represents a favorable starting point for developing scaffolded E1E2 vaccine candidates.

2. Results

i. Expression, Purification, and Immunization of Mice

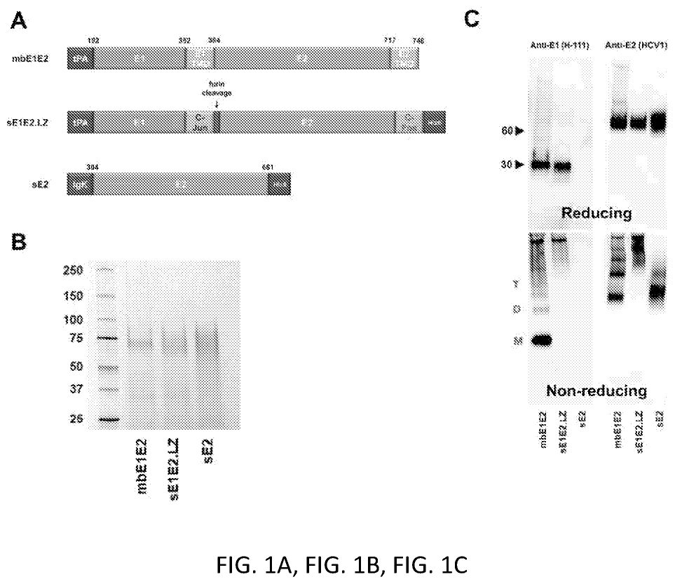

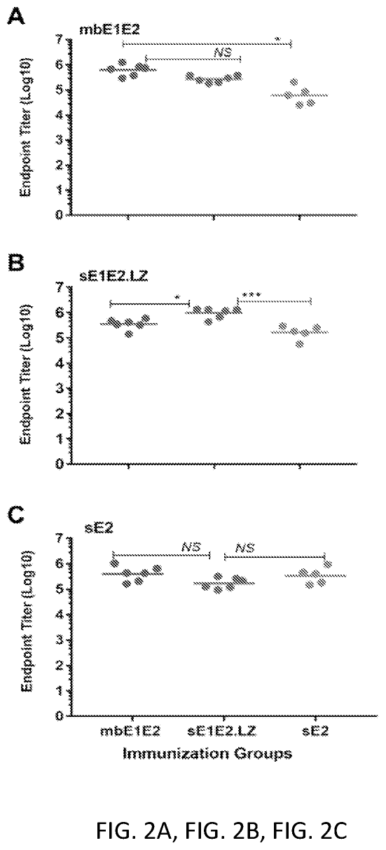

The design and in vivo assessment of a native-like secreted E1E2 heterodimeric glycoprotein assembly, sE1E2.LZ, was previously reported (Guest et al., Proc Natl Acad Sci USA 118 (2021)). Those results showed that sE1E2.LZ elicits robust neutralizing antibodies in vivo against pseudoparticles representing the homologous virus (H77C). To build on those promising results, a comparative assessment of neutralization breadth was performed and the polyclonal response to key conserved regions on E1E2 were assessed. To compare and evaluate the antigenicity and immunogenicity of sE1E2.LZ (Guest et al., Proc Natl Acad Sci USA 118 (2021)) in vivo, a study was conducted in which CD1 mice were immunized with purified mbE1E2, sE1E2.LZ, and sE2 (HCV E2 residues 384-661). Using the methods described previously (Guest et al., Proc Natl Acad Sci USA 118 (2021)), the three constructs were cloned, expressed, and purified, and SDS-PAGE and Western blot analyses performed to confirm the quality and quantity of antigen prior to formulation and injection into mice (FIG. 1). Three groups of mice (n=6 per group) were immunized with mbE1E2, sE1E2.LZ, and sE2, which were formulated into nano-scale size supramolecular assemblies with a polyphosphazene adjuvant (PCPP-R) (Andrianov et al., Mol Pharm (2020)), (Andrianov et al., ACS Appl Bio Mater 3, 3187-3195 (2020)), (Andrianov, et al., J Control Release 329, 299-315 (2021)). Blood samples were collected prior to each vaccination on days 0 (pre-bleed), (Bowen, et al., Nature 436, 946-952 (2005)), (Houghton, et al., Immunol Rev 239, 99-108 (2011)), and (Vieyres et al., Viruses 6, 1149-1187 (2014)) with a terminal bleed on day 56.

ii. Evaluation of Anti-E1E2 Serological Responses by ELISA

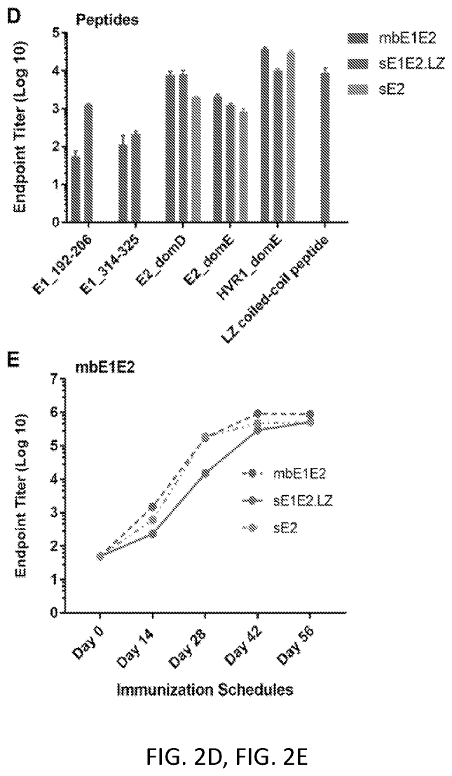

Day 56 serum samples from the three groups of mice were individually tested for anti-E1E2 antibody titers in which the ELISA plates were coated with mbE1E2 (FIG. 2A), sE1E2.LZ (FIG. 2B), or sE2 (FIG. 2C). As shown, sera from mice immunized with sE1E2.LZ were able to induce an anti-E1E2-specific response comparable to mice immunized with mbE1E2 or sE2. Because the E1E2 transmembrane regions were replaced by regions of the human c-Jun/c-Fos leucine zipper, biotinylated peptides from c-Fos and c-Jun were used to evaluate the degree of antibody responses to the c-Jun/c-Fos heterodimer scaffold by ELISA, Pierce et al., J Virol 94 (2020), 2 μg/mL of c-Jun/c-Fos peptides were mixed and coated on streptavidin plates. Endpoint titer values indicate that sE1E2.LZ induced a specific anti-Jun/Fos peptide response, and no detectable binding was observed in the mbE1E2 and sE2 immunized groups (FIG. 2D). Dimerization of the mixed c-Jun/c-Fos peptides were confirmed using circular dichroism spectroscopy. To further evaluate epitope-specific E1E2 antibody responses, peptides representing E2 antigenic domain D, E2 domain E, E2 hypervariable region one (HVR1) and domain E (i.e. combined), the E1 N-terminus, and an E1 ectodomain nAb epitope were synthesized and the relative ELISA responses were compared to the leucine zipper peptides across the three antigen groups. These peptides were chosen to provide an approximate baseline reactivity to epitopes that elicit antibodies that exhibit some neutralization potency in either E1 or E2, along with a peptide that contains a known immunodominant decoy epitope (HVR1). Within the sE1E2.LZ group, sera exhibited the strongest relative responses to peptides corresponding to the LZ scaffold, followed by E2 HVR1 and E2 domain D (FIG. 2D). Across the three groups, sera from sE1E2.LZ-immunized mice exhibited nearly identical responses to peptides corresponding to the E1 ectodomain nAb epitope and E2 domains D and E compared to sera from mice immunized with mbE1E2 and sE2. Remarkably, sera from sE1E2.LZ immunized mice showed an 11-fold higher response to a peptide corresponding to the E1 N-terminus and a 3- to 4-fold lower response to a peptide corresponding to the E2 decoy epitope HVR1 and domain E, relative to sera from mice immunized with mbE1E2 and sE2. Finally, pooled sera from each group were used to examine the kinetics of the anti-HCV E1E2 antibody response by assessing the overall response at each collection point. As shown in FIG. 2E, the antibody responses can be detected beginning on day 14 after the primary immunization among all three groups. The anti-E1E2 specific antibody titers reached a peak at day 42 and day 56, with similar overall titers for all three groups.

iii. Evaluation of Broadly Neutralizing Antibody Responses by Competition Inhibition Analysis

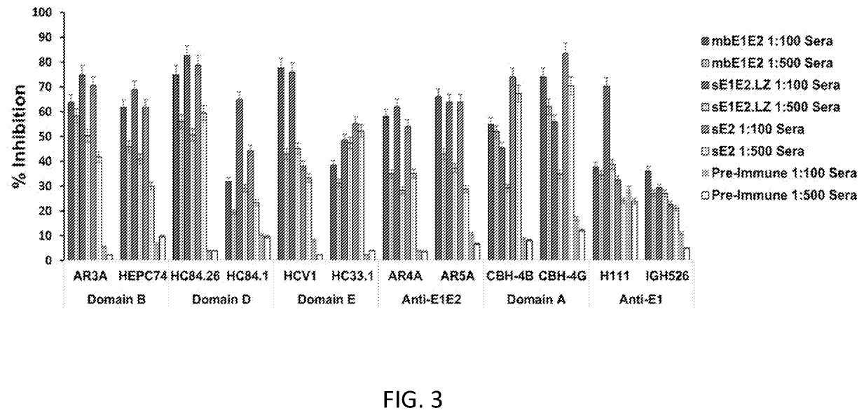

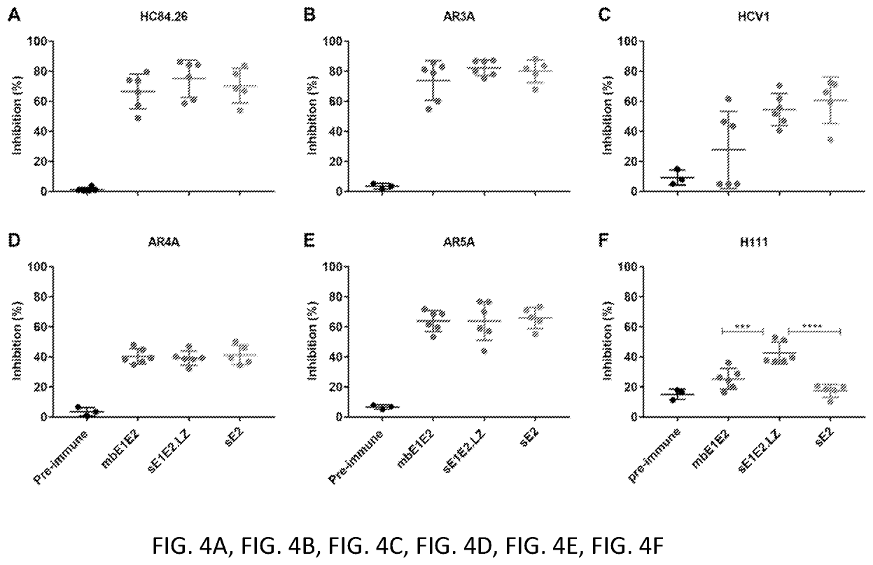

The relative magnitude of domain-specific serological responses to conserved, continuous and discontinuous epitopes were analyzed by competition inhibition ELISA using a panel of broadly neutralizing human monoclonal antibodies (HMAbs) derived from HCV-infected individuals (Giang et al., Proc Natl Acad Sci USA 109, 6205-6210 (2012)), (Pierce et al., Proc Natl Acad Sci USA 113, E6946-E6954 (2016)), (Kong et al., J Mol Biol 427, 2617-2628 (2015)), (Keck et al., J Virol 78, 7257-7263 (2004)), (Broering et al., J Virol 83, 12473-12482 (2009)), (Owsianka et al., J Virol 79, 11095-11104 (2005)). Pooled sera (day 56) from each group were used to compete with a pair of HMAbs from the following antigenic domains of E2: domain B (AR3A/HEPC74), domain D (HC84.26.WH.5DL/HC84.1), and domain E (HCV1/HC33.3); to the E1E2 heterodimer, AR4A and AR5A; to E1-specific antibodies, H-111 and IGH526, and to non-neutralizing E2 antibodies (CBH-4B, CBH-4G) (FIG. 3). While sera of all immunized mice were able to compete for the binding to E1E2 specific antibodies, sE1E2.LZ immunized mice showed nearly identical or stronger inhibitory activities in competing with antibodies corresponding to domain B (AR3A, HEPC74), domain D (HC84.26.WH.5DL, HC84.1), domain E (HC33.1, HCV1), E1E2 heterodimer (AR4A and AR5A), and E1 (H-111). In contrast, sera from mice immunized with sE2 and mbE1E2 had higher level of competition with the non-neutralizing antibodies, CBH-4B and CBH-4G. In order to further analyze the epitope-specific responses, competition ELISA was performed using individual mouse serum (day 56) on a select group of antibodies for statistical comparison (FIG. 4). Based on these results, there was a trend toward a higher level of competition in sE1E2.LZ immunized mice but no statistically significant difference among the groups, except the anti-E1 antibody, H-111. While cohorts immunized with mbE1E2 and sE1E2.LZ elicited antibodies corresponding to the E1E2 heterodimer antibodies, AR4A and AR5A, the serum from the sE2 immunized group also showed competition with these antibodies. This result is consistent with previous studies from the Law group (Giang et al., Proc Natl Acad Sci USA 109, 6205-6210 (2012)) in which they showed that AR5A competes with the E2 domain C antibody, CBH-7, for E1E2 binding. However, AR4A does not compete with CBH-7 and binds E1E2 utilizing D698 as a key binding residue in the highly conserved E2 membrane proximal external region (MPER). It is plausible that polyclonal E2-specific antibodies would compete with AR4A via steric hindrance or shared binding residues near the E1-E2 interface.

iv. Induction of Broadly Neutralizing Antibody Responses

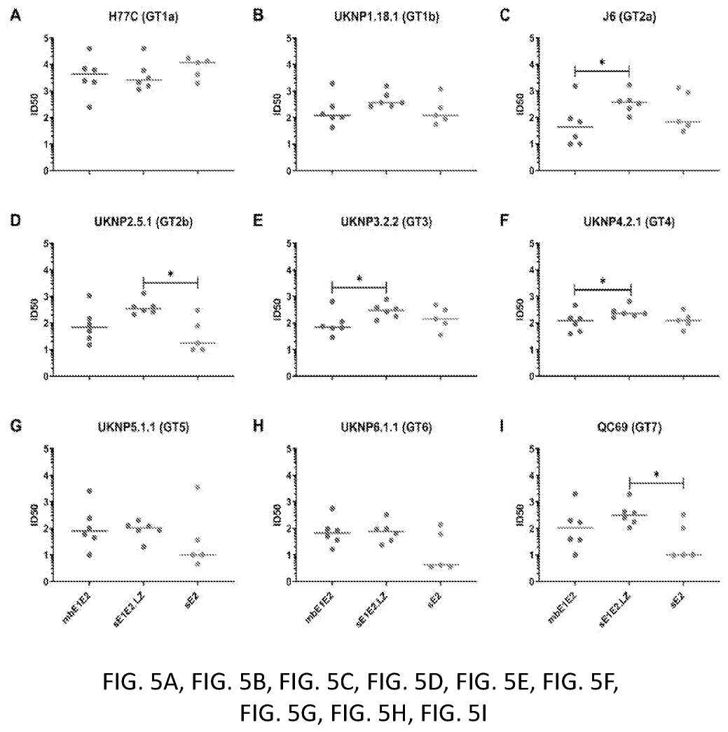

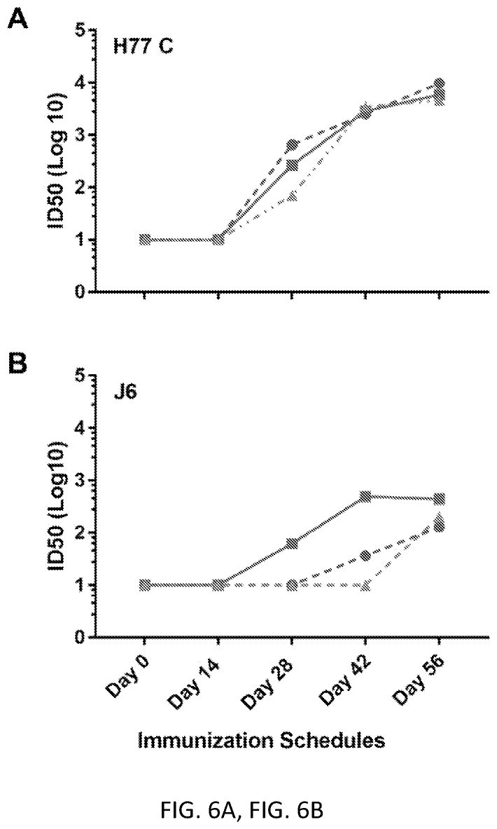

The ability of mbE1E2, sE1E2.LZ, and sE2 immunized mice sera to inhibit HCV infection in vitro was tested against a panel of HCVpp covering the structural proteins of the major HCV genotypes. HCVpp packaged with the E1E2 glycoproteins of seven antigenically distinct HCV genotypes (GT), GT1a (H77C, AF011751), GT1b (UKNP1.18.1), GT2a (J6), GT2b (UKNP2.5.1), GT3 (UKNP3.2.2), GT4 (UKNP4.2.1), GT5 (UKNP5.1.1), GT6 (UKNP6.1.1) and GT7 (QC69 YP_009272536.1) were produced in HEK293T cells (SI Appendix and [(Midgard et al., J Hepatol 64, 1020-1026 (2016)]) and used for neutralization assays (FIG. 5 and Table 1). Pre-immune and day 56 serum samples were used at two-fold serial dilutions from 1:64 to 1:8,192 and inhibition values (ID50) are expressed as the serum dilution level corresponding to 50% neutralization (ID50). The highest levels of neutralization were detected against homologous H77C HCVpp (GT1a) by all three immunized groups. No statistically significant differences were found among these three groups against HCVpp GT1a. Statistically significant higher ID50 values were observed with sE1E2.LZ sera, relative to mbE1E2 or sE2 sera, against heterologous genotypes GT2a (FIG. 5C), GT2b (FIG. 5D), GT3 (FIG. 5E), GT4 (FIG. 5F) and GT7 (FIG. 5I). In addition, sera from the sE1E2.LZ immunized group showed an identical or higher trend of neutralization for GT1b (FIG. 5B), GT5 (FIG. 5G) and GT6 (FIG. 5H). Neutralization activities were also assessed using time-point collected pooled sera at day 0, day 14, day 28, day 42 and day 56 (FIGS. 6A and B). Similar ID50 titers were identified in the homologous GT1a neutralization among three groups at each time-point. In the heterologous GT2a (J6) neutralization assay, sE1E2.LZ immunized mice showed measurable ID50 values at the last three time points, whereas neutralization was only detectable at day 42 for mbE1E2-immunized mice and day 56 for sE2-immunized mice. The sE1E2.LZ immunized group was the only one to exhibit neutralization activity at day 28 (prime and one boost), despite the fact that the anti-E1E2 antibody endpoint titers were similar across the three groups on that day (FIG. 2E), indicating a qualitative difference in the neutralization capacity of the polyclonal antibody response in mice immunized with sE1E2.LZ.

TABLE 1
Comparison of ID50 between HCVcc (pooled
serum) and HCVpp (mean ID50)

| HCV Genotypes | | ID50 | | |
|---|---|---|---|---|
| Genotypes | Isolates | mbE1E2 | sE1E2.LZ | sE2 |
| GT1a | H77C (HCVpp) | 9,607 | 9,032 | 9,739 |
| | H77C (HCVcc) | 141 | 741 | 562 |
| GT1b | UKNP1.20.3 (HCVpp) | 311 | 284 | 122 |
| | J4 (HCVcc) | 177 | 154 | 426 |
| *GT2a | J6 (HCVpp) | 294 | 533 | 468 |
| | Con1/jc1 (HCVcc) | 23 | 199 | 30 |
| *GT2b | UKNP2.5.1 (HCVpp) | 233 | 489 | 83 |
| | J8 (HCVcc) | 115 | 363 | 550 |
| *GT3 | UKNP3.2.2 (HCVpp) | 163 | 344 | 212 |
| | S52 (HCVcc) | 0 | 218 | 0 |
| *GT4 | UKNP4.2.1 (HCVpp) | 157 | 288 | 153 |
| | ED43 (HCVcc) | 95 | 645 | 112 |
| *GT5 | UKNP5.1.1 (HCVpp) | 504 | 107 | 715 |
| | SA13 (HCVcc) | 1,698 | 1,000 | 660 |
| \hat{ }GT6 | UKNP6.1.1 (HCVpp) | 140 | 107 | 42 |
| | HK (HCVcc) | 229 | 251 | 776 |
| *GT7 | QC69 (HCVpp) | 410 | 544 | 92 |
| | QC69 (HCVcc) | 62 | 794 | 19 |
| GT1b | UKNP1.18.1 (HCVpp) | 429 | 589 | 337 |
| | — | — | — | — |
| GT2b | UKNP2.4.1 (HCVpp) | 297 | 433 | 222 |
| | — | | | |

v. Assessment of Homologous Neutralization and Breadth Using the HCVcc System

To assess the efficacy of the hyperimmune sera from the vaccinated mice to block entry of infectious HCV, in vitro neutralization assays were performed using antigenically diverse cell culture derived HCV (HCVcc). The development of the genotype 2a JFH1 cell culture system (Wakita et al., Nat Med 11, 791-796 (2005)), and the more efficient J6/JFH1 system with the Core-NS2 region from another 2a isolate (Lindenbach et al., Science 309, 623-626 (2005)), has enabled the study of the entire viral life-cycle in vitro. Subsequent generation of intergenotypic chimeras harboring the structural proteins of antigenically diverse HCV genotypes has been very useful to assessing the breadth of neutralizing antibody responses to the virus. Bicistronic versions of H77C(1a)/JFH (T2700C, A4080T), Con1 (1b)/Jc1 (G2833C, T2910C, A4274G, A6558G, A7136C), J4(1b)/JFH (T2996C, A4827T), J6(2a)/JFH1, J8(2b)/JFH, ED43(4a)/JFH1 (A2819G, A3269T), SA13(5a)/JFH1 (C3405G, A3696G), HK(6a)/JFH (T1389C/A1590C) and QC69(7a)/JFH (T2985C, C8421T), (Scheel et al., Proc Natl Acad Sci USA 105, 997-1002 (2008)), (Jensen et al., J Infect Dis 198, 1756-1765 (2008)), (Gottwein et al., Hepatology 49, 364-377 (2009)), (Gottwein et al., Gastroenterology 133, 1614-1626 (2007)), (Gottwein et al., J Virol 84, 5277-5293 (2010)) expressing Gaussia luciferase (Gluc) were used. These genomes are replication competent in Huh7.5 cells and produce infectious virions. These genomes have been used to determine the in vitro neutralization capacity of bnAbs in mouse sera (de Jong et al., Sci Transl Med 6, 254ra129 (2014)).

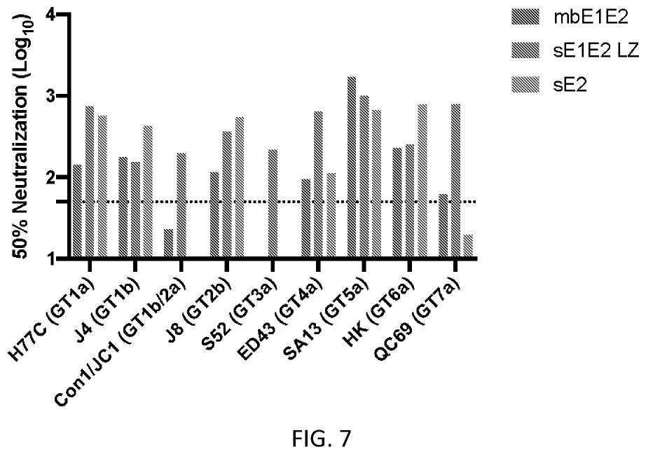

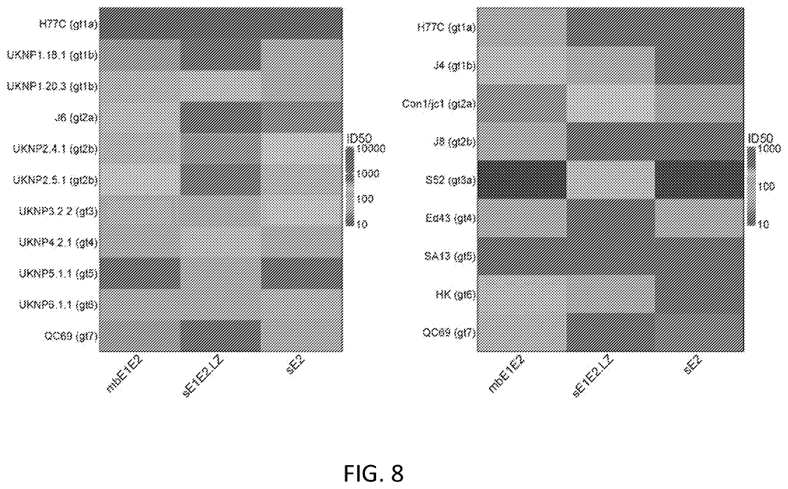

Pooled sera collected 56 days following immunization with mbE1E2, sE1E2.LZ or sE2 inhibited infections with the HCV intergenotypic chimeras with varying efficiencies depending both on the antigen and HCV genotype (FIGS. 7 and S3, Table 1). Sera derived from mbE1E2 vaccinated mice neutralized most efficiently the SA13 (GT5a) strain and slightly less efficiently, H77C (1a), J4 (1b), J8 (2b), ED43 (4a) and HK (6a). sE1E2.LZ serum showed high neutralization activity against all strains. sE2 serum showed high neutralization activity against H77C, J4, J8, SA13, and HK strains, and moderate or low neutralization activity against the ED43 strain. Thus, immunization by sE1E2.LZ and sE2 showed broad neutralization activity compared to full-length mbE1E2, and sE1E2.LZ in particular induced neutralization activity against all genotypes. These results indicate that broadly neutralizing antibodies were induced efficiently by the soluble glycoprotein design, sE1E2.LZ. To compare breadth of nAb responses elicited by mbE1E2, sE1E2.LZ, and sE2 measured by HCVpp and HCVcc systems, group ID50s were represented on a heatmap (FIG. 8). This clearly shows that sE1E2.LZ elicits broader neutralizing antibody responses than mbE1E2 and sE2 in mice, and supports the use of this class of scaffold for HCV vaccine development.

3. Discussion

A major challenge in developing an E1E2-based vaccine is producing homogeneous amounts of this complex membrane-associated protein in large quantities that reflects the native form found on the surface of the virus. Part of the difficulty stems from the fact that mbE1E2 undergoes a complex folding and processing pathway in which E1 and E2 mutually assist each other in achieving their native forms (Falson et al., J Virol 89, 10333-10346 (2015)), (Brazzoli et al., Virology 332, 438-453 (2005)), (Dubuisson et al., J Virol 70, 778-786 (1996)). An additional complication arises due to the membrane anchoring TMDs on E1 and E2, which makes membrane extraction required for mbE1E2 purification and sets an inherent limit on the amount of protein that can be produced per volume of cell culture. Recent efforts have made strides in liberating E1E2 from the membrane (Cao et al., PLoS Pathog 15, e1007759 (2019)), (Guest et al., Proc Natl Acad Sci USA 118 (2021)), (Ruwona et al., J Virol 88, 10459-10471 (2014)) and heterodimeric coiled-coil leucine zipper scaffolded secreted E1E2 (sE1E2.LZ) that retains native-like antigenicity and elicits neutralizing mAbs in mice were developed (Guest et al., Proc Natl Acad Sci USA 118 (2021)). In this study, the quality of sE1E2.LZ as an immunogen was assessed.

Based on the immunological response to sE1E2.LZ in a mouse model observed here as well as the previous biophysical characterization of sE1E22.LZ, the soluble heterodimeric coiled coil appears to be a bona fide functional replacement for the E1 and E2 TMDs and thus this platform provides an opportunity for further development of a soluble E1E2-based vaccine candidate. In particular, the overall antibody titers elicited by sE1E2.LZ were equal or superior to those elicited by mbE1E2 or sE2 (FIG. 2). Moreover, sE1E2/LZ was competent to elicit antibodies that target important neutralizing domains (B, D, E, E1E2) to the same extent as its membrane-bound counterpart (FIG. 3 and FIG. 4). Domains B and D are of particular importance for vaccine development because they elicit broadly-neutralizing antibodies (Keck et al., J Virol 82, 6061-6066 (2008)), (Keck et al., PLoS Pathog 8, e1002653 (2012)) and domain D in particular has a low propensity to accumulate mutants that allow viral escape (Keck et al., PLoS Pathog 10, e1004297 (2014)). Finally, the antibodies elicited by sE1E2.LZ are broadly neutralizing (FIGS. 5-8, Table 1). These properties of sE1E2.LZ persist despite the fact that it is purified in a manner that disadvantages it relative to mbE1E2. mbE1E2 is purified using an HC84.26.WH.5DL immunoaffinity column which, since HC82.26.WH.5DL is both conformation-specific and affinity matured (Keck et al., Hepatology 64, 1922-1933 (2016)), selects a population of mbE1E2 that has its domain D conformationally intact. sE1E2.LZ is purified using immobilized metal affinity chromatography which is insensitive to the integrity of neutralizing epitopes. It should be noted that sE2 is purified in the same manner, yet the neutralization breadth of sE2-immunized mice is not superior to that of mbE1E2. Using the leucine zipper scaffold as a starting point, additional stabilization of important neutralizing domains as a next step can result in an improved E1E2-based immunogen. Structural data pertaining to the leucine-zipper scaffolded sE1E2s would greatly accelerate such efforts. Another area for potential development is the scaffold itself. In FIG. 2D, a significant immunological response to the leucine zipper scaffold was observed. Since c-Fos and c-Jun are of human origin, incorporation of structurally-homologous scaffolds that are either of bacterial origin or rationally-designed and lack any sequence homology with human proteins is an important next step. The leucine zipper was chosen as a scaffold in part because the structure is well-characterized, making such a transition potentially straightforward.

Given the potential of this approach, it is important to consider the possible origins of improved neutralization breadth as these considerations will inform future designs. One advantage of the sE1E2.LZ platform is that it maintains neutralizing epitopes on E1, E2, and those that require the E1E2 complex in a soluble antigen. That these epitopes are intact is borne out by both previous biochemical analysis and the immunological response observed here. An additional factor that might contribute to increased neutralization breadth is lower immunoreactivity to non-neutralizing epitopes. Based on peptide ELISA data (FIG. 2D), sera from sE1E2.LZ-immunized mice exhibit 3- to 4-fold lower reactivity to a peptide containing the sequence of HVR1. HVR1 is an immunodominant region in patients infected with HCV (Farci et al., Proc Natl Acad Sci USA 93, 15394-15399 (1996)), (Shimizu et al., J Virol 68, 1494-1500 (1994)). As such, HVR1 provides many opportunities for viral escape as the region readily undergoes sequences changes during the course of an infection (von Hahn et al., Gastroenterology 132, 667-678 (2007)). Moreover, of the three antigens, sE1E2.LZ exhibits the weakest competition with non-neutralizing antigenic domain A mAbs in competition ELISA experiments. This indicates that among the polyclonal sera, those from sE1E2.LZ-immunized mice contain fewer mAbs that recognize domain A than sera from either mbE1E2 or sE2. sE2-immunized mouse sera contained the most domain A mAbs, consistent with the previous observation that sE2 binds CBH-4D and CBH-4G (Hadlock et al., J Virol 74, 10407-10416 (2000)) 15- to 30-fold tighter than sE1E2.LZ or mbE1E2 (Guest et al., Proc Natl Acad Sci USA 118 (2021)). A final potential contributor to increased neutralization breadth is increased homogeneity of the sE1E2.LZ antigen relative to mbE1E2. Previous biophysical analysis indicated that, while sE1E2.LZ is not a single, homogeneous species in solution, it is more homogeneous than mbE1E2 (Guest et al., Proc Natl Acad Sci USA 118 (2021)). It is possible that cellular quality checks on the secreted complex, such as the ER-associated degradation (ERAD) pathway, contribute to homogeneity.

Perhaps differences in the pathways that check the quality of membrane-bound versus secreted proteins (Bernasconi, et al., J Cell Biol 188, 223-235 (2010)), combined with the fact that mbE1E2 extracted from the membrane is likely to be a mix of proteins at various stages of the quality control pathways results in a more heterogeneous mbE1E2 preparation. For sE1E2.LZ, only protein that has completed the checks by the ERAD will be secreted from cells and ultimately purified, thereby limiting the number of species in solution.

In summary, the immunological response to the sE1E2.LZ validates the heterodimeric coiled coil leucine zipper scaffold as a platform for rational design of E1E2 immunogens capable of eliciting broadly neutralizing antibodies outside of a membrane or detergent environment. A number of successful structure-based vaccine designs for variable viruses such as influenza (Impagliazzo et al., Science 349, 1301-1306 (2015)), (Yassine et al., Nat Med 21, 1065-1070 (2015)), HIV (de Taeye et al., Cell 163, 1702-1715 (2015)), (Kulp et al., Nat Commun 8, 1655 (2017)), and RSV (Joyce et al., Nat Struct Mol Biol 23, 811-820 (2016)), Correia et al., Nature 507, 201-206 (2014)) where rationally designed immunogens optimize presentation of key conserved epitopes or stabilize conformations or assembly of the envelope glycoproteins. Such studies have been relatively limited for HCV glycoproteins compared with those from other viruses, in terms of design strategies employed and number of designs tested. Moreover, these efforts have largely been limited to the E2 ectodomain alone. Since the effect of design changes observed in the isolated E2 ectodomain might not translate directly in the context of the E1E2 heterodimer, having a validated, native-like secreted E1E2 will allow a more thorough exploration of rationally-designed E1E2 vaccine candidates. Finally, validation of the leucine zipper platform allows the use of high yield production systems that were previously only available for sE2 production, thereby making the transition to eventual clinical scale manufacturing of E1E2 vaccine antigens more feasible.

4. Materials and Methods

i. Plasmid Construction

In order to express the proteins of membrane-bound E1E2 (mbE1E2), the native-like and secreted form of E1E2 (sE1E2.LZ) and the secreted E2 (sE2) (HCV E2 residues 384-661), the human codon optimized cDNA sequences encoding the proteins of mbE1E2, sE1E2.LZ and sE2 were synthesized by GenScript and then cloned into pCDNA3.1 (+) and pSecTag2 respectively as described in the previous study (WHO (2017) Global Hepatitis Report 2017). The tissue plasminogen activator (tPA) leader sequence was used to replace the native lead sequences in the pCDNA3.1-based mbE1E2 and sE1E2 constructs, and the signal peptides from the mouse Ig kappa-chain (IgK) was used for pSecTag2-based sE2 construct. A C-terminal 6×His tag was added to both soluble sE1E2.LZ and sE2 constructs. In the sE1E2.LZ construct, the transmembrane domains (TMDs) of E1E2 were replaced by human c-Fos/c-Jun leucine zipper. A hexaarginine furin cleavage site was also incorporated between E1 and E2 to facilitate polyprotein processing.

ii. Protein Expression and Purification

Expression of recombinant mbE1E2, sE1E2.LZ and sE2 were performed in a transient expression in human Expi293 cells using the Expi293 Expression System by following the manufacturer's protocols (Thermo Fisher). Briefly, Expi293 cells were cultured in Expi293 Expression Medium in the shaker incubator at 37° C., with 120 rpm and 8% CO2. When the cells reached a density of 2.0×106 cells/mL, Expi293 cells were transfected using proper amounts of plasmid DNA. For the furin-cleavable polyprotein expression, sE1E2.LZ construct was co-transfected with the furin construct (kindly provided by Dr. Yuxing Li) at a 2:1 ratio. Culture supernatants of sE1E2.LZ and sE2 were harvested at 72 hours after transfection, clarified by centrifugation at 10,000 rpm for 10 min, and filtered by 0.22 μm filters. Protein was then purified from the supernatant by sequential HisTrap Ni2+-NTA and Superdex 200 size exclusion chromatography (SEC) as described in the previous paper (WHO (2017) Global Hepatitis Report 2017, H. Midgard et al., J Hepatol 64, 1020-1026 (2016)). Expi293 cells transfected with recombinant mbE1E2 were collected 72 hours after transfection and the cell pellets were lysed using 1% NP-9 cell lysis buffer (WHO (2017) Global Hepatitis Report 2017). Recombinant mbE1E2 was then purified by sequential Fractogel EMD TMAE (Millipore), Fractogel EMD SO3- (Millipore). HC84.26 immunoaffinity (100), and Galanthus Nivalis Lectin (GNL, Vector Laboratories) affinity chromatography as described previously (WHO (2017) Global Hepatitis Report 2017).

iii. SDS-PAGE and Western Blot

Purified proteins of mbE1E2, sE1E2.LZ and sE2 were separated by a precast, 4-20% Mini-PROTEAN TGX stain-free gels on a Mini-PROTEAN Tetra cell electrophoresis instrument (Bio-Rad Laboratories). In reducing conditions, each sample was incubated with loading dye (4× Laemmli buffer+10% J3-mercaptoethanol) (Bio-Rad) and heated to 95° C. In non-reducing conditions, each sample was incubated with Laemmli buffer and heated to 37° C. For western blot detection, the purified protein samples on SDS-PAGE were transferred onto Trans-Blot Turbo Mini nitrocellulose membranes (Bio-Rad Laboratories). The membranes were then probed using the anti-HCV E2 mAb HCV1 at 5 μg/mL and anti-HCV E1 mAb H-111 at 10 μg/mL followed by detection using a secondary goat anti-human IgG-HRP conjugate (Invitrogen) at a 1:5,000 dilution and the Western ECL substrate (Bio-Rad). All gels were imaged using the ChemiDoc system (BioRad).

iv. Animal Immunization

CD1 mice were purchased from Charles River Laboratories. Prior to immunization, sE2 and E1E2 (mbE1E2 and sE1E2.LZ) antigens were formulated with polyphosphazene adjuvant as described in previous studies (Andrianov et al., Mol Pharm (2020)), Andrianov et al., ACS Appl Bio Mater 3, 3187-3195 (2020)). In brief, 50 μg PCPP was formulated with 25 μg resiquimod, R848 in PBS (pH 7.4) to form the PCPP-R adjuvant. The resulting supramolecular complex (PCPP-R) was formulated with either E1E2 (70 μg for prime or 15 μg for boost immunization) or sE2 antigen (50 μg for prime or 10 μg for boost immunization), with antigen amounts selected to ensure approximate molar equivalence of E2 in the vaccines. Dynamic light scattering (DLS) was used to confirm the absence of aggregation in adjuvanted formulations. Groups of six female CD-1 mice, age 7 to 9 weeks, were immunized via the intraperitoneal (IP) route on day 14, day 28 and day 42.). Unvaccinated mice served as a control for later analysis. Blood samples were collected prior to each vaccination on days 0 (pre-bleed), 14, 28, 42 and a terminal bleeding on day 56. The blood samples were processed for serum by centrifugation and stored at −80° C. until analysis was performed.

v. ELISAs for Serum Antibody Detection

ELISA was performed to measure HCV E1E2-specific antibody responses in immunized mouse serum. 96-well plates (MaxiSorp, Thermo Fisher) were coated overnight with 5 μg/mL Galanthus Nivalis Lectin (Vector Laboratories) at 4° C. The next day, plates were washed with PBS containing 0.05% Tween 20 and coated with 200 ng/well antigens of mbE1E2, sE1E2.LZ and sE2 at 4° C. After overnight incubation, plates were washed with PBS containing 0.05% Tween 20 and blocked with Pierce™ Protein-Free Blocking Buffer (Thermo Fisher) for 1 hour, and serially diluted mice sera samples were then added to the plates and incubated for another hour. The binding of HCV E1E2-specific antibodies was detected by a 1:5,000 dilution of HRP-conjugated anti-mouse IgG secondary antibody (Abcam) with TMB substrates (Bio-Rad Laboratories, Hercules, CA). Absorbance values at 450 nm (SpectraMax M3 microplate reader) were used to determine endpoint titers, which were calculated by curve fitting in GraphPad Prism software and defined as four times the highest absorbance value of pre-immune sera. Significance comparison was performed using Kruskal-Wallis one-way ANOVA.

For peptide ELISA, 100 μl of biotinylated peptides (2 μg/mL) were coated on the Well-Coated™ Streptavidin plates (G-Biosciences) overnight at 4° C. Peptides included in this study were c-Fos (LTDTLQAETDQLEDKKSALQTEIANLLKEKEKLEFILAAY, SEQ ID NO:9) and c-Jun (RIARLEEKVKTLKAQNSELASTANMLREQVAQLKQKVMNY, SEQ ID NO:8), along with peptides representing E2 domain D (NTGWLAGLFYQHK, SEQ ID NO:54), E2 domain E (NIQLINTNGSWHINS, SEQ ID NO:55), E2 hypervariable region one (HVR1) and domain E (ETHVTGGSAGRTTAGLVGLLTPGAKQNIQLINTNGSWHIN, SEQ ID NO:56), the E1 N-terminus (YQVRNSSGLYHVTND, SEQ ID NO:57) and an E1 ectodomain nAb epitope (TGHRMAWDMMMN, SEQ ID NO:58). After washing with PBS containing 0.05% Tween 20 and blocking with Pierce™ Protein-Free Blocking Buffer, serial diluted pooled mice sera, ranging from 1:150 to 1:328,050, were incubated at 37° C. for 1 hour and detected by ELISA as described above.

vi. Competition ELISA

The ability of antibodies in immunized mouse sera to compete with both conformation-dependent and linear HCV E1E2-specific HMAbs was assessed by ELISA. The antibodies used for these experiments include AR3A and HEPC74 (domain B), HC84.26 and HC84.1 (domain D), HCV1 and HC33.1 (domain E), AR4A and AR5A (anti-E1E2), CBH-4G and CBH-4B (domain A) and and IGH526 (anti-E1). mbE1E2 was captured on GNA-coated microtiter plates at 4° C. for overnight. After blocking with Pierce™ Protein-Free Blocking Buffer (Thermo Fisher) for 1 hour followed by three-time washing using Pierce™ Protein-Free Blocking Buffer, diluted mouse antisera (terminal bleed) were added to each well and incubated for 1 hour at room temperature. After plates were washed with PBS containing 0.05% Tween 20, HCV E1E2-specific HMAbs were added at a concentration demonstrated previously to result in 70% of maximal binding and incubated for an additional hour. The HMAbs used for the competition ELISA were biotinylated using an EZ-Link NHS-PEO solid-phase biotinylation kit (Thermo Fisher). Bound biotinylated HMAb was detected using HRP-conjugated streptavidin (Abcam) at a dilution of 1:20,000. Absorbance was read at 450 nm using a SpectraMax M3 microplate reader. Percent inhibition values were calculated as the percentage of mAb binding relative to the mAb bound in the absence of serum.

vii. HCVpp Neutralization Assay

The human hepatoma cell line, Huh7, was maintained in the DMEM medium supplemented with 10% FBS and 1% non-essential amino acids (NEAA) (Thermo Fisher), and used as the target cell line for neutralization assays (1,10). To test sera and antibodies for neutralization, Huh7 cells were pre-seeded into 96-well plates at a density of 1×104 per well. The next day, the pseudoparticles were incubated with defined concentrations of mAbs and/or the heat-inactivated serum at indicated dilutions for 1 hour at 37° C., and then added to each well. After the plates were incubated in a CO2 incubator at 37° C. for 5 to 6 hours, the mixtures were replaced with fresh medium and then continued to incubate for 72 hours. After incubation, 100 μl Bright-Glo (Promega) was added to each well for 2 min at room temperature and the luciferase activity was measured using a FLUOstar Omega plate reader (BMG Labtech) with the MARS software. The 50% inhibitory concentration (IC50) titer was calculated as the mAbs concentration that caused a 50% reduction in relative light units (RLU) compared with pseudoparticles in the control wells. Neutralizing antibody (nAbs) titers in animal sera were reported as 50% inhibitory dilution (ID50) values. All values were calculated using a dose-response curve fit with nonlinear regression in GraphPad Prism. All experiments involving the use of pseudoparticles were performed under biosafety level 2 conditions.

viii. HCVcc Neutralization Assay

Two-fold dilutions were performed starting at 1:100 pre-immune pooled serum or 1:50 day 56 pooled serum. And HCVcc was mixed with diluted serum (final MOI=0.1) and incubated for 1 hour at 4° C. After the incubation, the serum and virus mixture was added onto Huh7.5 cells (kindly provided by Charles Rice, The Rockefeller University), plated on 96-well plate for 1 day, and cultured for 4 hours at 37° C. Thereafter, the inoculum was removed, cells washed with HBSS twice, and then the cells were cultured with DMEM containing 3% fetal bovine serum (FBS, Atlanta biologicals), nonessential amino acids (NEAA, 0.1 mM, Thermo Fisher scientific), HEPES (20 mM, Thermo Fisher scientific), polybrene (4 μg/mL, Sigma-Aldrich Chemie GmbH) and penicillin streptomycin for 72 hr at 37° C. After 72 hours, supernatants were collected and luciferase assay was performed following the manufacturer's protocol (GeneCopoeia Inc.). % Neutralization was calculated as relative luminescence units (RLU) from supernatant cultured without HCVcc nor serum was 100% neutralization and RLU from supernatant cultured with HCVcc without serum was 0% neutralization. The serum concentration of 50% Neutralization was calculated from the sigmoid curve (Prism 8).

ix. Statistical Analysis

The differences among group endpoint titers and group ID50 values were statistically compared using the nonparametric Kruskal-Wallis test with Dunn's multiple comparisons test. A p value of <0.05 was considered significant. All statistical analyses were performed using GraphPad Prism software.

### B. Soluble, Secreted E1E2 Antigen for Vaccinating Against the Hepatitis C Virus

1. Introduction

Hepatitis C virus (HCV) is a global disease burden, with an estimated 71 million people infected worldwide (1, 2). Roughly 75% of HCV infections become chronic (3-5), and in severe cases can result in cirrhosis or hepatocellular carcinoma (6). Viral infection can be cured at high rates by direct acting antivirals (DAAs), but multiple public health and financial barriers (7, 8), along with the possibility of reinfection or continued disease progression (7, 9, 10), have resulted in a continued rise in HCV infections. An HCV vaccine remains essential to proactively protect against viral spread, yet vaccine developments against the virus have been unsuccessful to date (11, 12). The challenges posed by HCV sequence diversity (12, 13), glycan shielding (14, 15), immunodominant non-neutralizing epitopes (16-19), and preparation of a homogeneous E1E2 antigen all contribute to the difficulty in generating protective B cell immune responses. Though multiple studies in chimpanzees and humans have used E1E2 formulations to induce a humoral immune response, their success in generating high titers of broadly neutralizing antibody (bnAb) responses has been limited (20). Optimization of E1E2 to improve its immunogenicity and elicitation of bnAbs through rational design may lead to an effective B cell based vaccine (21).

HCV envelope glycoproteins E1 and E2 form a heterodimer on the surface of the virion (22-24). Furthermore, E1E2 assembly has been proposed to form a trimer of heterodimers (25) mediated by hydrophobic C-terminal transmembrane domains (TMDs) (24, 26, 27) and interactions between E1 and E2 ectodomains (28-30). These glycoproteins are necessary for viral entry and infection, as E2 attaches to the CD81 and SR-B1 co-receptors as part of a multi-step entry process on the surface of hepatocytes (31-34). Neutralizing antibody responses to HCV infection target epitopes in E1, E2, or the E1E2 heterodimer (18, 35-40). Structural knowledge of bnAb antibody-antigen interactions, which often target E2 epitopes in distinct antigenic domains B, D, or E (18, 41, 42), can inform vaccine design efforts to induce bnAb responses against flexible HCV epitopes (43-45). E1E2 bnAbs, including AR4A, AR5A (46), and others recently identified (38), are not only among the most broadly neutralizing (35), but also represent E1E2 quaternary epitopes unique to antibody recognition of HCV.

Though much is known about bnAb responses to E1E2 glycoproteins, induction of B cell based immunity with a E1E2-based vaccine immunogen (47-49) has remained difficult. The inherent hydrophobicity of E1 and E2 transmembrane domains (TMDs) (24, 50) may impede uniform production of an immunogenic E1E2 heterodimer that could be utilized for both vaccine development and E1E2 structural studies. Although partial E1 and E2 structures have been determined (39, 51-54), many other enveloped viruses have structures of a complete and near-native glycoprotein assembly (55-59), providing a basis for rational vaccine design (60-62). Viral glycoproteins of influenza hemagglutinin (63), respiratory syncytial virus (RSV) (55), SARS-CoV-2 (64), and others (65, 66) have been stabilized in soluble form using a C-terminal attached foldon trimerization domain to facilitate assembly. HIV gp120-gp4l proteins have been designed as soluble SOSIP trimers in part by introducing a furin cleavage site to facilitate native-like assembly when cleaved by the enzyme (56, 67). Previously described E1E2 glycoprotein designs include covalently-linked E1 and E2 ectodomains (68, 69), E1E2 with transmembrane domains intact and an IgG Fc tag for purification (70), as well as E1 and E2 ectodomains with a cleavage site (68), which presented challenges for purification either due to intracellular expression or to high heterogeneity. Two recently described scaffolded E1E2 designs, while promising, have not been shown to engage mAbs that recognize the native E1E2 assembly, though they were engaged by E1-specific and E2-specific mAbs, as well as co-receptors that recognize E2 (71). Therefore, these presentations of E1E2 glycoproteins may not represent a native and immunogenic heterodimeric assembly, and thus their potential as vaccine candidates remains unclear.

Here, the design of a secreted E1E2 glycoprotein (sE1E2) that mimics both the antigenicity in vitro, and the immunogenicity in vivo, of the native heterodimer through the scaffolding of E1E2 ectodomains is described. In testing the designs, it was found that both replacing E1E2 TMDs with a leucine zipper scaffold and inserting a furin cleavage site between E1 and E2 enabled secretion and native-like sE1E2 assembly. The size, heterogeneity, antigenicity, and immunogenicity of this construct (identified as sE1E2.LZ) were assessed in comparison with full-length membrane-bound E1E2 (mbE1E2). sE1E2.LZ binds a broad panel of bnAbs to E2 and E1E2, as well as co-receptor CD81, providing evidence of assembly into a native-like heterodimer. An immunogenicity study indicated that sera of mice injected with sE1E2.LZ neutralize HCV pseudoparticles at levels comparable to sera from mice immunized with mbE1E2. This sE1E2 design is a novel form of the native E1E2 heterodimer that both improves upon current designs and represents a platform for structural characterization and engineering of additional HCV vaccine candidates.

2. Results

i. Design of sE1E2 Constructs

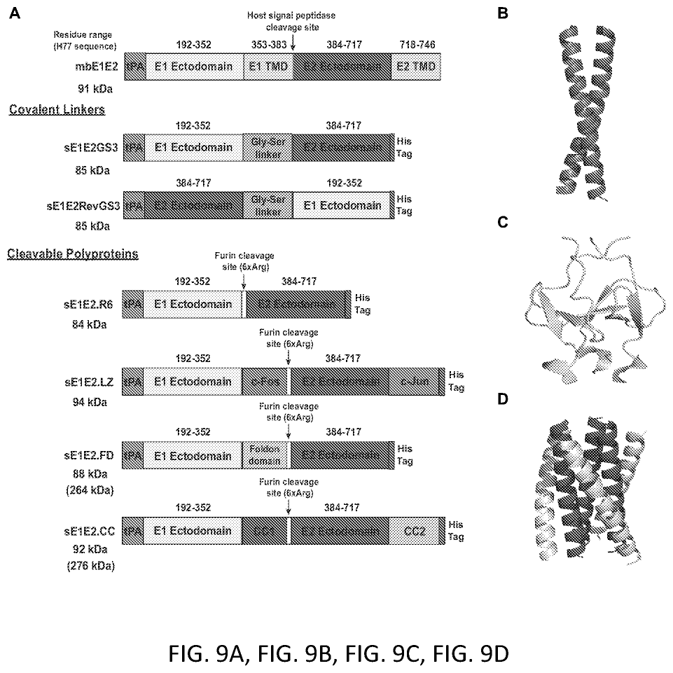

A set of secreted E1E2 (sE1E2) constructs were designed and screened to determine which type of scaffold might be suitable for development of a novel secreted heterodimer (FIG. 9A). Scaffolded sE1E2 constructs were constructed as cleavable polyproteins, and contain a six-arginine furin cleavage site, which was incorporated to facilitate E1E2 assembly similar to HIV SOSIP constructs (56). Each cleavable polyprotein replaces E1 and E2 TMDs with a self-assembling heterodimeric, homotrimeric, or heterohexameric scaffold designed to enforce E1E2 ectodomain assembly in the absence of a membrane anchor. In addition, all constructs replace the N-terminal wild-type signal peptide sequence with a modified version of the signal sequence from tissue plasminogen activator (tPA) (72) and include a C-terminal 6×His tag for purification.

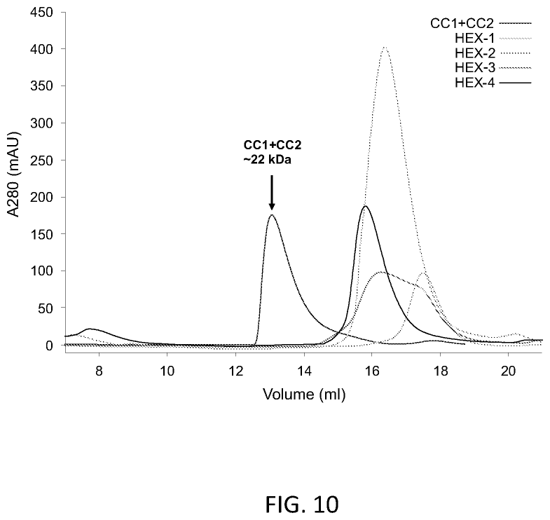

sE1E2.LZ used the human c-Fos/c-Jun leucine zipper, a coiled-coil obligate heterodimer with a known structure (PDB code 1FOS; FIG. 9B) (73), as a scaffold. The heterodimeric c-Fos/c-Jun leucine zipper has been used as a scaffold for expression of T cell receptors (74), making it a possible candidate for maintaining heterodimeric E1E2 in secreted form. sE1E2.FD replaced the E1 TMD with a foldon domain (FIG. 9C; PDB code: 4NCU) (75), a self-trimerizing protein that has been previously used to stabilize soluble assemblies of viral glycoprotein trimers (55, 76). This construct was designed to test whether enforcing E1 trimerization (25) would be sufficient to enable E1E2 ectodomain assembly. sE1E2.CC used a scaffold that was designed to self-assemble into a heterohexameric peptide complex, which would reflect the previously described model of the E1E2 TMD architecture (25) in a soluble form. The corresponding scaffold, CC1+CC2 (FIG. 9D), was designed de novo using the HBNet protocol of Rosetta protein modeling software (77). The structure of CC1+CC2 was not confirmed with experimental structural determination, however, it was included as a candidate scaffold given its putative hexameric assembly (FIG. 10). To examine the importance of including scaffolds in the absence of TMDs, a separate construct with a furin cleavage site but no scaffold was generated (sE1E2.R6). Two sE1E2 constructs with a covalent linker between ectodomains were also included. In sE1E2GS3, E1 and E2 ectodomains are linked by a 15 amino acid glycine-serine sequence, similar to a previously described sE1E2 construct (68). The construct sE1E2RevGS3 reverses the order of E1 and E2 ectodomains, testing whether altering the order of ectodomains in the context of a covalent fusion may improve E1E2 assembly, which could be affected by the currently unknown proximity of the N- and C-termini of the ectodomains in native E1E2.

ii. sE1E2.LZ Forms an Intact E1E2 Complex

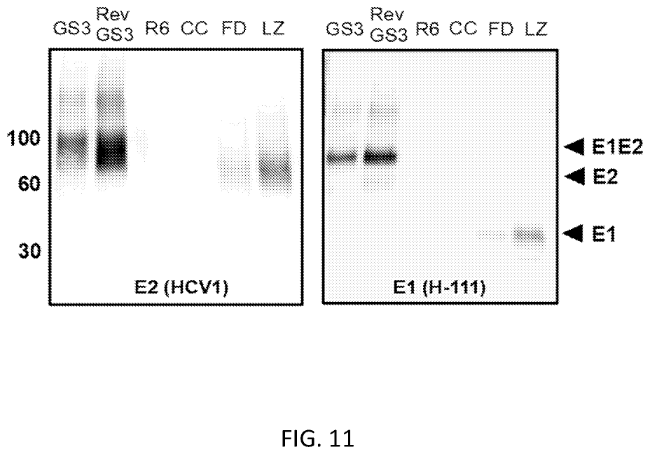

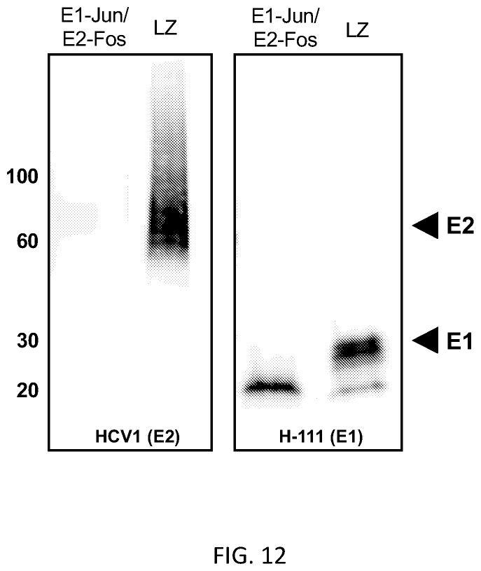

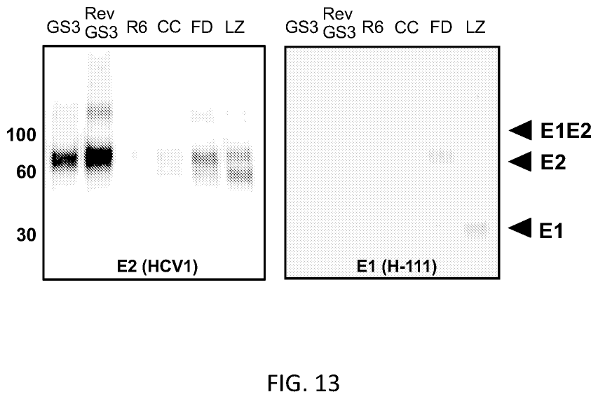

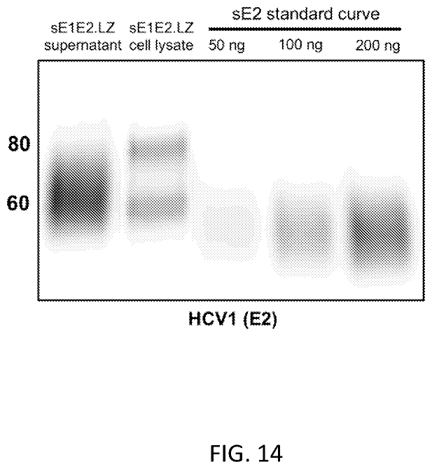

Each sE1E2 construct was expressed in mammalian cells, with cleavable polyproteins co-expressed with furin. To test for successful secretion of sE1E2, the presence of E1 and E2 ectodomains were probed for in the supernatant, using the E1 human monoclonal antibody (HMAb) H-111 (78) and the E2 HMAb HCV1 (79) in western blots. These antibodies bind to linear epitopes at or near the N-terminus of the E1 or E2 ectodomain, respectively. sE1E2.LZ was the only cleavable polyprotein design to show clear detection of both E1 and E2 in the supernatant (FIG. 11), though sE1E2.FD exhibited some secretion of E2. The scaffold-less sE1E2.R6 construct showed no secretion of sE1E2, consistent with previous results that E1 and E2 ectodomains alone do not form a stable complex (71). Expression of E1-Jun and E2-Fos constructs in trans without a furin cleavage site found secretion of E1-Jun, but minimal secretion of E2-Fos (FIG. 12). Collectively, these results determine that the combination of a furin cleavage site and leucine zipper scaffold enables secretion of the E1E2 complex. sE1E2GS3 and sE1E2RevGS3 showed high levels of E1 and E2 in supernatant, corroborating previous findings with a covalently-linked sE1E2 design that is similar to sE1E2GS3 which was likewise detected in the supernatant (68). In addition, if protein was expressed but not secreted was examined by probing for the presence of E1 and E2 in lysed cells (FIG. 13). sE1E2GS3 and sE1E2RevGS3 that was retained in cells migrated as smaller molecular weights than the corresponding secreted proteins, while sE1E2.FD and sE1E2.LZ exhibited multiple bands in E2 detection; both results can be indicative of incomplete processing or degradation of unsecreted protein. Though some sE1E2.LZ was detected intracellularly, approximately 90% of expressed sE1E2.LZ was secreted to the supernatant, as determined by a quantitative analysis comparing supernatant and cell lysate western blots probed with the anti-E2 HMAb HCV1 (FIG. 14). Based on these results, sE1E2.LZ was selected, as a cleaved scaffolded sE1E2 candidate, and sE1E2GS3, as a covalently linked sE1E2 candidate, for further characterization.

iii. Purification of sE1E2.LZ

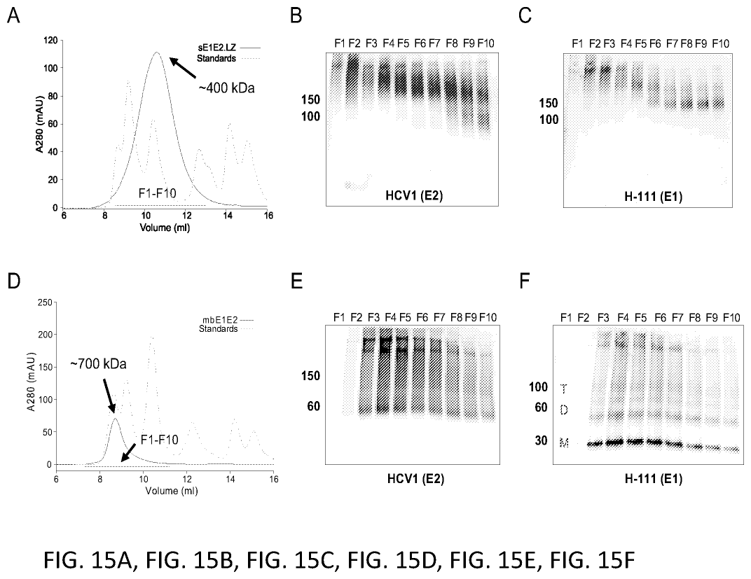

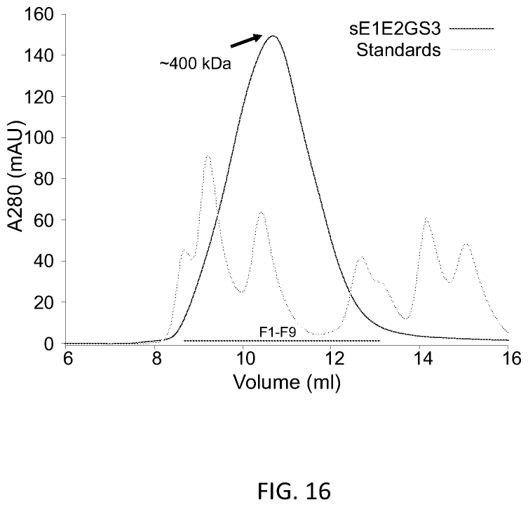

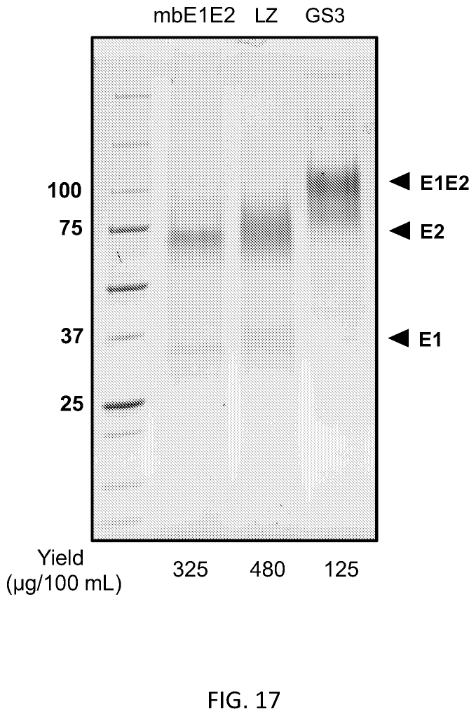

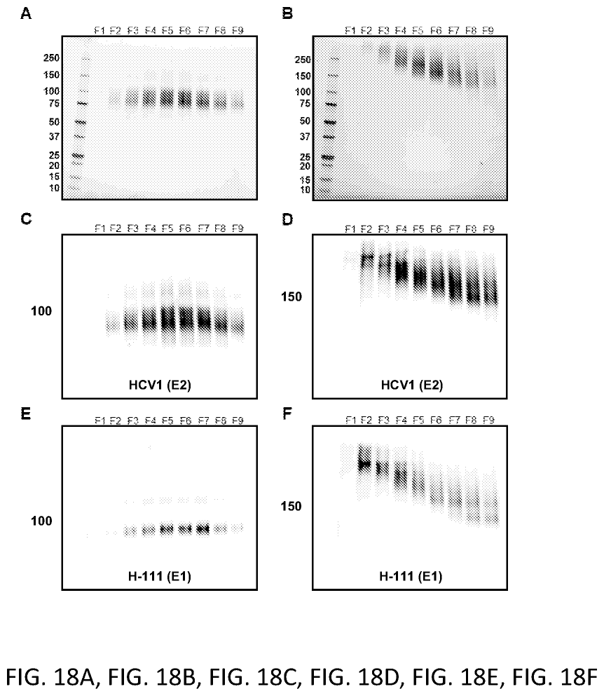

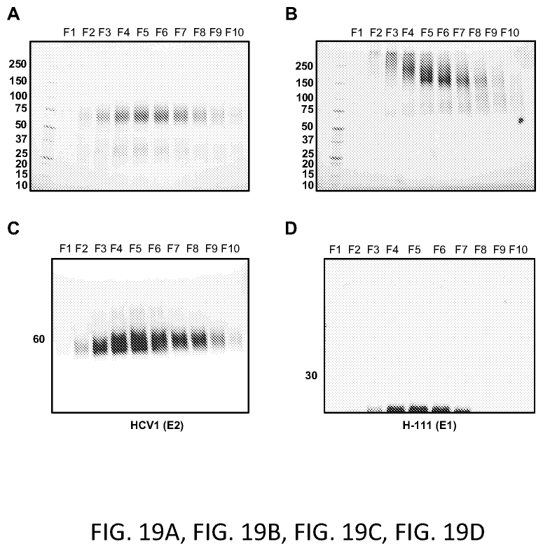

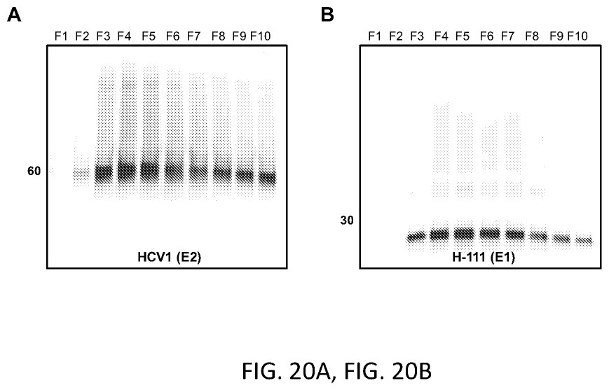

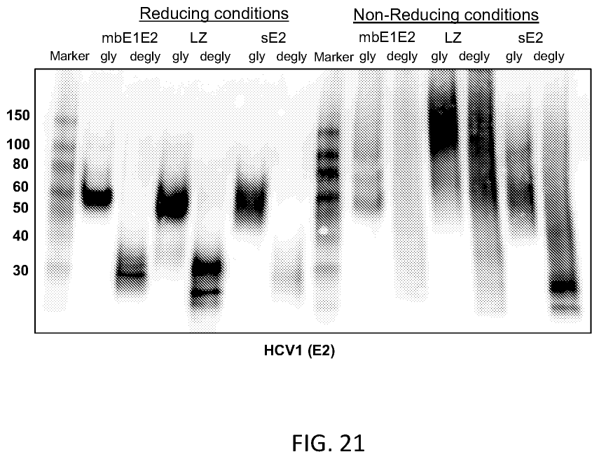

Both sE1E2.LZ and sE1E2GS3 were purified using immobilized metal affinity chromatography (IMAC), and then examined the molecular weight and heterogeneity of each construct with size exclusion chromatography (SEC) (FIG. 15A; FIG. 16). Expression and purification of all three constructs produced sufficiently pure protein for characterization, with sE1E2.LZ providing the highest yield at 480 μg/100 ml of transfected cells (FIG. 17). Both constructs eluted from the Superdex 200 column across a broad molecular weight range, with the peak for each estimated at approximately 400 kDa. The resultant SEC peaks were directly compared with the peak SEC fractions of purified mbE1E2 (FIG. 15D). Though sE1E2.LZ, along with sE1E2GS3, exhibited a broad peak in SEC, it eluted at a volume consistent with a molecular weight that is both smaller than mbE1E2, which eluted as a peak in void volume (approximately 700 kDa), and closer to the expected size of the heterodimeric assembly (94 kDa; FIG. 9). To further investigate the size distribution and heterogeneity of purified constructs, fractions eluted from SEC were examined under non-reducing conditions, using western blot for sE1E2.LZ (FIGS. 15B-15C), mbE1E2 (FIGS. 15E-15F), and sE1E2GS3 (FIG. 18D, FIG. 18F), and SDS-PAGE for sE1E2GS3 (FIG. 18B) and sE1E2.LZ (FIG. 19B). Both sE1E2.LZ and sE1E2GS3 SEC fractions showed two predominant species migrating in the range between 150 and 250 kDa when probed for E1 and E2 under non-reducing conditions, which is smaller than expected based on the SEC chromatographs but confirms the heterogeneity of each protein. mbE1E2 SEC fractions probed by western blot under non-reducing conditions showed a number of species including prominent bands corresponding to free E1 and E2 along with higher molecular weight aggregates. In addition, the anti-E1 non-reducing western blot shows a discrete bands corresponding to self-associating E1 dimers and trimers as observed previously (25), indicating that, while the purified protein is a heterogenous mixture, the mixture contains a significant population of natively-assembled E1E2. In contrast, under reducing conditions the E1 and E2 components migrated at the expected molecular weight for both sE1E2.LZ (FIG. 19) and mbE1E2 (FIG. 20) fractions, and at a molecular weight corresponding to covalently linked E1E2 in sE1E2GS3 (FIG. 18) fractions. The spread of the bands in SDS-PAGE and western blots is likely due to in part heterogeneity in glycoforms. To examine the contribution of glycosylation to observed size distributions, the purified proteins to PNGase F cleavage to remove the glycans was subjected. An examination of the deglycosylated proteins on a non-reducing western blot showed more species (FIG. 21), indicating that the heterogeneity in solution was observed for all constructs is dominated by another factor, possibly disulfide crosslinking or exchange. Although these results indicate that sE1E2.LZ is closer to expected size of a heterodimer than mbE1E2, the ranges of observed sizes led us to utilize more sensitive methods of characterization to examine molecular size and heterogeneity.

iv. Analytical Characterization of Heterogeneity in Solution

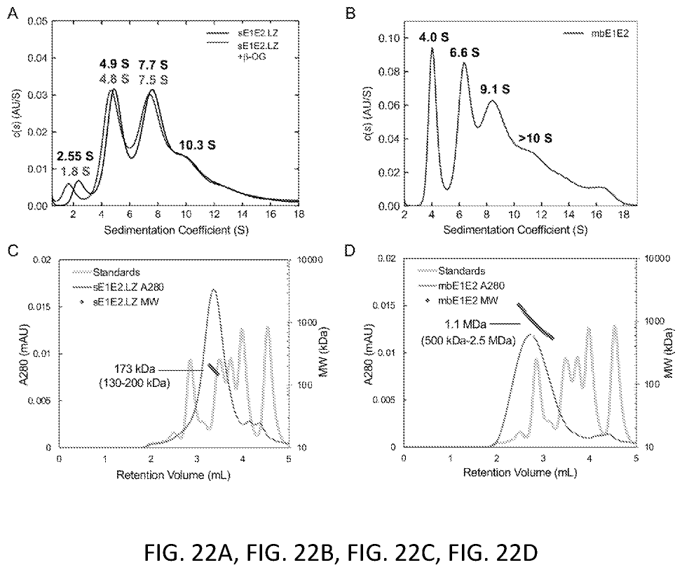

sE1E2.LZ and mbE1E2 purified constructs were also characterized using analytical ultracentrifugation (AUC), which can separate a mixture of protein populations more precisely than SEC. A comparison of AUC results offers further support that sE1E2.LZ is less heterogeneous than mbE1E2. AUC for sE1E2.LZ showed two prominent peaks between sedimentation coefficient (S) values 4.9 and 7.5, which are approximately consistent with a monomer and dimer of the sE1E2.LZ heterodimer, respectively, and resemble what was observed in the non-reducing western blot. To control for potential effects of 0.5% n-Octyl-β-D-Glucopyranoside (β-OG), a detergent required for mbE1E2 purification, a parallel AUC experiment was performed with sE1E2.LZ in the presence of 0.5% β-OG (FIG. 22A). The size distribution in that experiment closely matched that of the sample without β-OG, indicating that the detergent itself does not contribute to heterogeneity. mbE1E2 showed three large peaks between S values 4 and 9.1, indicating that mbE1E2 exhibits more heterogeneity than sE1E2.LZ (FIG. 22B). Furthermore, the peak with the highest intensity for mbE1E2 closely resembles the S value found for free E2. sE1E2.LZ by contrast shows no peak at that S value. Though sE1E2.LZ is not a uniform single species, it is a less complex mixture of E1E2 assemblies than mbE1E2.

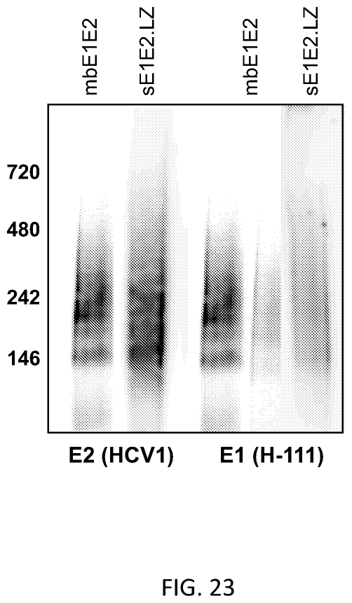

SEC with multi-angle light scattering (SEC-MALS) was used as another analytical technique to examine the heterogeneity and size of sE1E2.LZ. Since the presence of β-OG detergent had little to no effect on sE1E2.LZ in AUC, it was expected that an absence of β-OG would not affect analytical characterization of sE1E2.LZ in SEC-MALS. When compared with standards and analyzed by light scattering, sE1E2.LZ exhibited a single peak in SEC-MALS with an estimated molecular weight at peak center of 173 kDa, corresponding approximately to a dimer of the sE1E2.LZ heterodimer (FIG. 22C). This estimated size is generally consistent with the observed AUC peak around 7.5 S, though the breadth of the peak in SEC-MALS still suggests that sE1E2.LZ displays some heterogeneity in size, corresponding to 1-2 sE1E2.LZ heterodimers, in accordance with the two major peaks from AUC measurements. In SEC-MALS, mbE1E2 was characterized as a single, very broad peak with an estimated molecular weight of 1.1 MDa at peak center (FIG. 22D). The broad range of this peak identified mbE1E2 as a mixture containing a broad range of species, with approximately 5 to over 20 E1E2 heterodimers. Additionally, sE1E2.LZ was directly compared to mbE1E2 in a native western blot, showing differences in overall size (FIG. 23). In assessments by multiple analytical techniques, sE1E2.LZ forms a moderately heterogeneous mixture that is nonetheless smaller and closer to expected size than mbE1E2, representing a potentially improved immunogen for HCV vaccine development and a candidate for structural characterization. In addition, sE1E2.LZ does not require detergents for solubility, allowing for simpler formulations than mbE1E2.

v. sE1E2.LZ Exhibits Native-Like E1E2 Antigenicity and Robust Immunogenicity

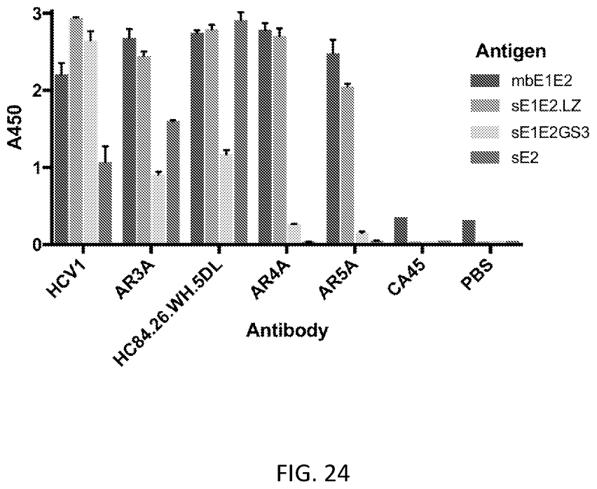

The native-like properties of sE1E2.LZ was also examined by measuring the binding affinities to a panel of bnAbs in comparison with secreted E2 ectodomain (sE2) and mbE1E2. Unlike the antibodies used in western blot, most bnAbs used for this analysis recognize conformational epitopes on E2 (41, 84, 85), and E1E2 (46). An ELISA was performed at one antibody concentration to compare mbE1E2 and sE1E2.LZ antibody reactivity, along with purified sE1E2GS3 and sE2. This screening was used to assess lack of reactivity by any of the constructs to conformationally sensitive antibodies, versus quantitative comparisons of affinities, which was undertaken later. The antibodies utilized were a representative panel of bnAbs to antigenic domain B, D, and E epitopes in E2 and the E1E2 bnAbs AR4A and AR5A (FIG. 24). At the tested antibody concentration (0.185 μg/ml), mbE1E2 and sE1E2.LZ exhibited similar binding levels for all antibodies. Importantly, sE1E2.LZ maintained reactivity to E1E2 bnAbs, providing evidence that this sE1E2 construct contains a soluble, native-like form of the E1E2 heterodimer. In contrast, sE1E2GS3 and sE2 showed little to no reactivity to AR4A and AR5A; this was not unexpected for sE2, which lacks key residues comprising the E1E2 bnAb epitopes (86). Based on the AR4A and AR5A binding results, the lack of E1-E2 cleavage or scaffold in sE1E2GS3 appears to lead to a severe disruption of native-like assembly, thus the focus was on sE1E2.LZ for subsequent characterization.

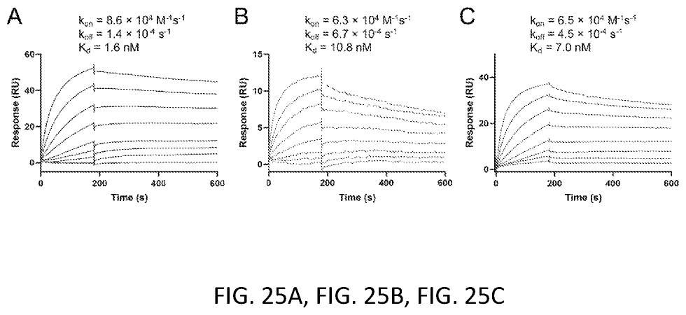

To confirm more precisely the initial measurements of bnAb reactivity, the affinity of sE1E2.LZ to a larger panel of HCV antibodies (Table 1) and CD81 (FIG. 25) was tested. Dissociation constants (Kds) were measured by dose-dependent ELISA to antibodies that recognize discrete epitopes of E2 (18) and E1E2 bnAbs. For comparison, the same analysis was performed for purified mbE1E2 and sE2. sE1E2.LZ and mbE1E2 showed similar affinities to almost all tested HCV human monoclonal antibodies (HMAbs), within a 2-3 fold difference. One notable exception was an 8-fold lower affinity of AR4A for sE1E2.LZ relative to mbE1E2. Although sE1E2.LZ maintained affinity to AR5A, a decrease in affinity to AR4A may stem from subtle differences in heterodimer assembly or dynamics when compared to mbE1E2, which may be difficult to elucidate without detailed structural characterization of the epitope. Regardless, AR4A binds sE1E2.LZ with nanomolar affinity (16 nM), indicating that the overall structure of the AR4A epitope and the E1E2 interface in that region are intact. In addition to measurements of binding to conformationally sensitive E2 and E1E2 HMAbs, binding to the CD81 receptor, which recognizes a region on the E2 ectodomain overlapping with epitopes for a number of bnAbs, was also tested (86). sE1E2.LZ showed robust binding to the large extracellular loop (LEL) of CD81 in surface plasmon resonance (10.8 nM; FIG. 25), establishing that this sE1E2 construct displays receptor binding critical for native HCV infection. While measured CD81-LEL KD values show comparable and in some cases higher affinity than corresponding glycoprotein affinities for antibodies in Table 2, due to the different experimental measurement methods, these results provide a comparison between antigens rather than a comparison between absolute glycoprotein affinities of receptor versus antibodies.

TABLE 2
Binding affinity of mbE1E2, sE1E2.LZ, and sE2 to a panel of
monoclonal antibodies measured by dose-dependent ELISA, with
standard error values shown for each affinity measurement.

| | | | | | | Standard | |
|---|---|---|---|---|---|---|---|
| | | K_{d} (nM) | | | | Error (nM) | |
| Antibody | Domain¹ | mbE1E2 | sE1E2.LZ | sE2 | mbE1E2 | sE1E2.LZ | sE2 |
| CBH-4D | A | 28 | 26 | 1 | 3.2 | 3.4 | 0.2 |
| CBH-4G | A | 7.8 | 18 | 0.5 | 2.3 | 3.1 | 0.3 |
| HC-1 | B | 1.5 | 2.9 | 3.6 | 0.06 | 0.5 | 0.4 |
| AM² | | | | | | | |
| HC-11 | B | 1.8 | 3.2 | 11 | 0.09 | 0.4 | 0.6 |
| CBH-7 | C | 1 | 1.7 | 0.3 | 0.1 | 0.1 | 0.04 |
| HC84.24 | D | 0.5 | 1.3 | 0.7 | 0.07 | 0.1 | 0.1 |
| HC84.26 | D | 1.2 | 2.6 | 0.4 | 0.03 | 0.4 | 0.1 |
| HC33.1 | E | 3.8 | 0.9 | 1.9 | 0.3 | 0.09 | 0.2 |
| HCV1 | E | 9.8 | 3.5 | 6.2 | 0.3 | 0.2 | 0.3 |
| AR4A | E1E2 | 2.3 | 16 | — | 0.2 | 1.5 | — |
| AR5A | E1E2 | 1.5 | 1.7 | — | 0.2 | 0.2 | — |

“—” denotes no binding detected
¹Antigenic domain on E2 targeted by antibody (A-E), as previously described (108). “E1E2” denotes antibodies that target the E1E2 heterodimer.
²Affinity-matured HC-1 antibody, as previously described (109).

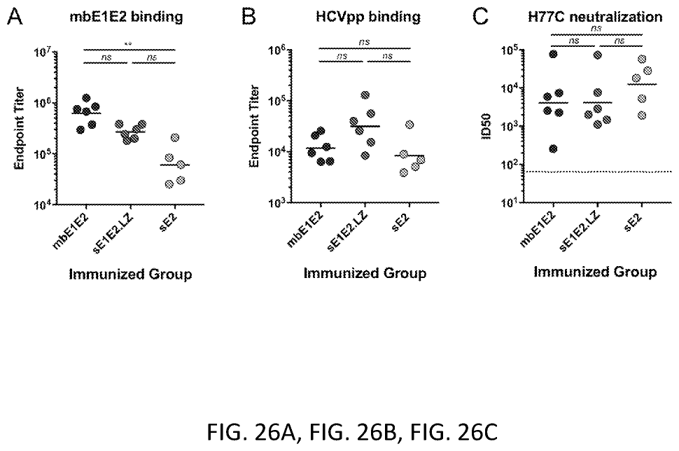

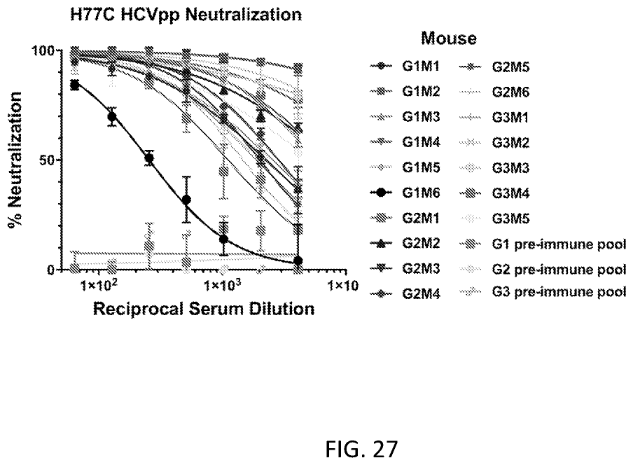

After confirming the native-like antigenicity of sE1E2.LZ, the native-like properties of sE1E2.LZ were tested in vivo, to determine whether it will elicit antibodies that effectively recognize HCV and inhibit infection. Mice were immunized with either mbE1E2, sE1E2.LZ, or sF2 and tested for the presence of antibodies that target E1E2 and neutralize the virus (FIG. 26). sE1E2.LZ elicited anti-mbE1E2 antibody responses that were similar to responses from mbE1E2-immunized mice, while serum binding of mbE1E2 from sF2-immunized mice was lower, in particular compared with the mbE1E2-immunized group (p<0.01) (FIG. 26A). Binding of immunized sera to H77-pseudotyped HCV pseudoparticles (HCVpp) was also tested for all groups (FIG. 26B), and while mean serum titer was highest for the sE1E2.LZ group, there were no significant differences found between immunized group titers based on non-parametric (Kruskal-Wallis) assessment. Serum neutralization of H77C HCVpp was tested for all groups to assess for elicitation of neutralizing antibodies that target the homologous virus (FIG. 26C). Testing of pre-immune sera for background neutralization showed no detectable HCVpp neutralization (FIG. 27). sE1E2.LZ-immunized sera showed robust neutralization of HCVpp, with neutralization titers (ID50s) that showed no significant difference from mbE1E2-immunized and sE2-immunized groups. This initial test of sE1E2.LZ immunogenicity shows that this secreted E1E2 construct is able to induce an antibody response comparable to mbE1E2 and sE2 that can recognize homologous E1E2 on the surface of HCVpp and neutralize the virus.

3. Discussion

The development and characterization of a native-like E1E2 antigen containing a leucine zipper scaffold offers a proof of principle platform for designing E1E2 vaccine antigens within a soluble and secreted backbone. Exploration of this scaffold approach for the production of E1E2 from other HCV genotypes is warranted, as sE1E2.LZ was only designed using the H77C sequence. E2 ectodomains from other strains have been characterized structurally (39, 54, 87), and the E1E2 sequences of those strains could be targets for sE1E2.LZ backbone expression and characterization. However, strain-specific sequence changes may affect sE1E2.LZ secretion, as differences in E1 and E2 stalk regions could modulate assembly and export from cellular components (88, 89). In addition, further studies of sE1E2 secretion may shed light on cellular factors that facilitate efficient sE1E2 assembly, which could then be used either to improve production levels or to examine mechanisms of viral assembly and secretion.

There are several avenues for subsequent design and optimization of the sE1E2.LZ platform. As a potential vaccine immunogen, the human leucine zipper of sE1E2.LZ poses potential problems related to immunizing humans with human protein sequences (90, 91). As the c-Jun/c-Fos leucine zipper is structurally defined at high resolution, this can be used as a template for identification of heterodimeric leucine zipper structures from non-human proteins or de novo designs of synthetic leucine zipper scaffolds. Furthermore, although the CC1+CC2 sE1E2 design (sE1E2.CC) did not yield appreciable secretion, it is possible that alternative heterohexameric scaffolds, possibly generated using c-Jun/c-Fos leucine zipper structure as a subunit, could promote stable E1E2 assembly. Finally, recent studies have shown that cage-like protein nanoparticles can provide scaffolds for viral glycoproteins such as RSV F (92, 93) and influenza hemagglutinin (57). A nanoparticle recapitulating the c-Jun/c-Fos leucine zipper structure as attachment points could be identified or designed to present sE1E2 in a similar nanoparticle format. Binding to E1E2-specific antibodies, such as AR4A and AR5A, is particularly important for validation of scaffolded E1E2 antigens. Since sE1E2.LZ exhibited slightly impaired binding to AR4A, new designed or synthetic scaffolds may provide an opportunity to improve upon the human leucine zipper scaffold by matching or exceeding wild-type binding to E1E2-specific antibodies. High-resolution structural characterization of sE1E2.LZ or subsequent designs, enabled by effective secretion and purification of this native-like assembly, can permit an improved view of the determinants of E1E2 assembly and support structure-based modifications to enhance assembly and stability.

Although sE1E2.LZ was observed as closer to expected size of a heterodimer than mbE1E2, extensive analytical characterization indicated a likely mix of heterodimers and higher-order oligomers. This degree of sample heterogeneity has been found during purification of previous soluble construct designs, both with a covalent linker (68) and a designed heterodimeric scaffold (71). Although glycoform heterogeneity is apparent in both constructs, these results indicate that it is not the primary source of observed oligomerization. Instead, these constructs demonstrate that removing the heterodimer from its natural membrane-attached environment does not preclude formation of large assemblies. The E2 ectodomain likely plays a large role in aggregation via additional hydrophobic interactions or disulfide crosslinking, as its ectodomain contains conserved and surface-exposed tyrosines, tryptophans, and cysteines (18). These residues are critical for co-receptor interactions (36, 94), proper ectodomain folding, and assembly (86, 88), but could readily mediate E1E2 aggregation without TMDs present. Self-association of E2 ectodomains has also been noted previously (95), offering additional support for the propensity of soluble E2 to exhibit crosslinking.

In summary, replacing the native TMDs of E1 and E2 with a leucine zipper scaffold provides support that this approach can be used to develop a native-like, antigenically and immunogenically intact E1E2 complex without requiring a membrane or detergent environment. The design and validation of additional scaffolds that adopt dimeric, trimeric, or heterohexameric quaternary structures could elucidate key determinants of E1E2 complex assembly, another area of research that has been hindered by membrane association of E1E2. In addition, this scaffold approach could serve as a platform to study how the substantial genetic diversity of HCV translates to structural diversity and envelope glycoprotein dynamics, and how structural and dynamic changes, including “open” and “closed” envelope glycoprotein states, may promote immune evasion, as noted by recent work (97). Finally, in addition to their use in structural characterization, designed soluble E1E2 complexes with functional TMD replacements that retain all essential structural properties can serve as an integral component of rational vaccine design.

4. Materials and Methods

i. Protein Expression

For expression of recombinant soluble HCV E2 (sE2), the sequence from isolate H77C (GenBank accession number AF011751; residues 384-661) was cloned into the pSecTag2 vector (Invitrogen), and expressed in mammalian (Expi293F) cells as described previously (98). The mbE1E2 and sE1E2 DNA coding sequences were synthesized with a modified tPA signal peptide (72) at the N-terminus. All E1E2 sequences were cloned into the vector pcDNA3.1+ at the cloning sites of KpnI/NotI (GenScript). Furin sequence DNA was cloned into the vector pcDNA3.1 and was a gift from Dr. Yuxing Li (University of Maryland IBBR). All sE1E2 constructs and mbE1E2 were transfected with ExpiFectamine 293 into Expi293F cells for expression (Invitrogen). Cleavable polyprotein constructs were co-transfected with the furin construct at a 2:1 ratio. A clone for mammalian expression of CD81 large extracellular loop (LEL), containing N-terminal tPA signal sequence and C-terminal twin Strep tag, was provided by Dr. Joe Grove (University College London). CD81-LEL was expressed through transient transfection in Expi293F cells (ThermoFisher Scientific).

ii. Antibodies

Monoclonal antibodies used in ELISA and binding studies were produced as previously described (84, 99, 100), with the exception of AR4A and AR5A, which were kindly provided by Dr. Mansun Law (Scripps Research Institute).

iii. Protein Purification and Size Exclusion Chromatography

sE2 glycoprotein was purified from cell supernatant as described previously (98). Culture supernatant of sE1E2.LZ and sE1E2GS3 was purified by immobilized metal affinity chromatography (IMAC) with separate HiTrap chelating HP Ni2+-NTA columns (Cytiva). Expressed mbE1E2 was extracted from cell membranes using 1% NP-9 and purified via sequential Fractogel EMD TMAE (Millipore), Fractogel EMD SO3-(Millipore). HC84.26 immunoaffinity (101), and Galanthus Nivalis Lectin (GNL, Vector Laboratories) affinity chromatography. Sample concentration prior to size exclusion chromatography was conducted with 15 ml Amicon Ultra 3 kDa centrifugal filters (Millipore Sigma). sE1E2.LZ, sE1E2GS3, and mbE1E2 were purified using a Superdex 200 Increase 10/300 column (Cytiva). sE1E2.LZ and sE1E2GS3 were equilibrated with 1× Phosphate-buffered saline (PBS; 10 mM sodium phosphate+150 mM NaCl) pH 7, while mbE1E2 was equilibrated in Tris-buffered saline (TBS; 25 mM Tris-HCl+150 mM NaCl) pH 7.5+0.5% n-Octyl-β-D-Glucopyranoside (Anatrace). Size exclusion fractions of 500 μl were collected on AKTA FPLC (Cytiva). Molecular weight standards from the high molecular weight (HMW) calibration kit (Cytiva) were compared to purified sE1E2.LZ, sE1E2GS3, and mbE1E2.

iv. Size Exclusion Chromatography Coupled to Multiple Angle Light Scattering (SEC-MALS)

For SEC-MALS, a UHPLC system (Vanquish Flex, Thermo Fisher) was coupled to MALS (DAWN HELEOS-II, Wyatt) and Refractive Index (Optilab T-rEX, Wyatt) detectors. Separations were performed using a WTC-050N5 column (Wyatt) equilibrated in PBS for sE1E2.LZ or in TBS+0.5% β-OG for mbE1E2, with a flow rate of 0.3 mL/min and sample injection volumes of 25 μL. Molar mass analysis was performed using the software ASTRA 7.1.3 (Wyatt) using refractive index as a concentration source.

v. SDS-PAGE and Western Blot

SDS-PAGE and western blot experiments were conducted with 12-well stain-free gels (Bio-Rad), with total protein detected using a stain-free imager (Bio-Rad). For SDS-PAGE, Precision Plus Unstained Protein Standards (Bio-Rad) were used as a molecular weight marker. E2 was detected in western Blot with HCV1 (79) as the primary antibody. E1 was detected in western Blot with H-111 as the primary antibody (78). In reducing conditions, each sample was incubated with loading dye (4× Laemmli buffer+10% β-mercaptoethanol) (Bio-Rad) and heated to 95° C., with the exception of mbE1E2, which was heated to 37° C. In non-reducing conditions, each sample was incubated with a Laemmli buffer and heated to 37° C. For western blots, stain-free gels were transferred to a turbo mini 0.2 μm nitrocellulose membrane (Bio-Rad) using the trans-blot turbo transfer system (Bio-Rad). Supersignal Molecular Weight Protein Ladder (ThermoFisher Scientific) was used as a marker for western blots. 10× concentration of supernatant for E1 western blots was conducted in 0.5 mL Amicon Ultra 3 kDa centrifugal filters (Millipore Sigma). Cell lysates of sE1E2.LZ and mbE1E2 were collected by centrifugation of 1 ml transfected cell suspension and extraction from cell membranes with 1% NP-9. For native western blots, 15-well NativePAGE Novex 4-16% Bis-Tris protein gels (ThermoFisher Scientific) were transferred to a turbo mini 0.2 μm PVDF membrane (Bio-Rad) using the same transfer system. NativeMark unstained protein standard (Invitrogen) was used as a molecular weight marker for native gels. To deglycosylate sE1E2.LZ, mbE1E2, and sE2 in non-denaturing conditions, 3 μg of each protein was mixed with 2 μl PNGase F enzyme (New England Biolabs), then incubated at 37° C. for 24 hours before western blot preparation. Proteins were detected with goat anti-human IgG HRP conjugate (Invitrogen) and clarity western ECL substrate (Bio-Rad). All gels were imaged using the ChemiDoc system (Bio-Rad).

vi. Analytical Ultracentrifugation

Sedimentation velocity (SV) experiments were performed at 20° C. using a ProteomeLab Beckman XL-A with absorbance optical system and a 4-hole An60-Ti rotor (Beckman Coulter). For sE1E2.LZ, the sample and reference sectors of the dual-sector charcoal-filled epon centerpieces were loaded with 390 μL protein in PBS, pH 7.4 with or without 0.5% β-OG, and 400 μL buffer. For mbE1E2, the sample and reference sectors of the dual-sector charcoal-filled epon centerpieces were loaded with 390 μL protein in TBS+0.5% β-OG, and 400 μL buffer. The cells were centrifuged at 40 krpm and the absorbance data were collected at 280 nm in a continuous mode with a step size of 0.003 cm and a single reading per step to obtain linear signals of <1.25 absorbance units. Sedimentation coefficients were calculated from SV profiles using the program SEDFIT (102). The continuous c(s) distributions were calculated assuming a direct sedimentation boundary model with maximum entry regularization at a confidence level of 1 standard deviation. The density and viscosity of buffers at 20° C. and 4° C. were calculated using SEDNTERP (103). The c(s) distribution profiles were prepared with the program GUSSI (C. A. Brautigam, Univ. of Texas Southwestern Medical Center).

vii. Enzyme-Linked Immunosorbent Assay (ELISA)

HCV HMAb binding to mbE1E2, sE1E2.LZ, sE1E2GS3, and sE2 were evaluated and quantitated by ELISA. 96-well microplates (MaxiSorp, Thermo Fisher, Waltham, MA) were coated with 5 μg/mL Galanthus Nivalis Lectin (Vector Laboratories, Burlingame, CA) overnight, and purified mbE1E2, sE1E2.LZ, sE1E2.GS3 and sE2 was then added to the plates at 2 ug/ml. After the plates were washed with PBS and 0.05% Tween 20, and blocked by Pierce™ Protein-Free (PBS) Blocking Buffer (Thermo Fisher, Waltham, MA), the mAbs were tested in duplicate at 3-fold serial dilution starting at 100 ug/ml. The binding was detected by 1:5000 dilutions of HRP-conjugated anti-human IgG secondary antibody (Invitrogen, Carlsbad, CA) with TMB substrate (Bio-Rad Laboratories, Hercules, CA). The absorbance was read at 450 nm using a SpectraMax MS microplate reader (Molecular Devices, San Jose, CA). For ELISA measurements of immunized murine sera, endpoint titers were calculated by curve fitting in GraphPad Prism software, with endpoint OD defined as four times the mean absorbance value of Day 0 sera.

viii. Determination of Antibody Affinity by Quantitative ELISA

ELISA were performed as described (84) to compare antibody affinity to sE1E2.LZ, mbE1E2, and sE2. Briefly, plates were developed by coating wells with 500 ng of GNA and blocking with 2.5% non-fat dry milk and 2.5% normal goat serum. Purified sE1E2.LZ, mbE1E2, and sE2 at 5 μg/ml were captured by GNA onto the plate and later bound by a range of 0.01-200 μg/ml of antibody. Bound antibodies were detected by incubation with alkaline phosphatase-conjugated goat anti-human IgG (Promega), followed by incubation with p-nitrophenyl phosphate for color development. Absorbance was measured at 405 nm and 570 nm. The assay was carried out in triplicate in three independent assays for each HMAb. The data were analyzed by nonlinear regression to measure antibody dissociation constants (Kd) and binding potential (optical density at 405 nm) using Graphpad Prism software, and standard deviation values were calculated using the three independent affinity measurements.

ix. Surface Plasmon Resonance

SPR analysis was performed using a Biacore™ T200 system (Cytiva) and HBS-EP+ buffer was used as a sample and running buffer. The analysis temperature and sample compartment were set to 25° C. mbE1E2, sE2, and sE1E2.LZ were immobilized on Series S CM5 chips using the Amine Coupling Kit per the manufacturer's instructions. Antigen capture levels were adjusted to yield approximately 1000 RU for the kinetic experiments. Purified CD81-LEL was injected over reference and active flow cells, applying a single cycle kinetics procedure using twelve concentrations. Data were fitted to a 1:1 binding model using Biacore™ T200 Evaluation Software 2.0. As one concentration series was used to calculate binding parameters, no standard errors were calculated for those values.

x. Animal Immunization

CD-1 mice were purchased from Charles River Laboratories. Prior to immunization, sE2 and E1E2 antigens were formulated with polyphosphazene PCPP-R adjuvant (104). Poly[di(carboxylatophenoxy)phosphazene], PCPP (50 μg, molecular weight 800,000 Da) (105) was formulated with resiquimod, R848 (25 μg) in PBS (pH 7.4) to prepare PCPP-R as described previously (104). The resulting formulation was mixed with E1E2 antigen (70 μg for prime or 15 μg for boost immunization). The absence of aggregation in adjuvanted formulations was confirmed by dynamic light scattering (DLS): single peak, z-average hydrodynamic diameter—60 nm. The formation of antigen-PCPP-R complex was confirmed by asymmetric flow field flow fractionation (AF4) as described previously (106). On scheduled vaccination days, groups of 6 female mice, age 7-9 weeks, were injected via the intraperitoneal (IP) route with a 50 μg E1E2 prime (day 0) and boosted with 10 μg E1E2 on days 7, 14, 28, and 42. Blood samples were collected prior to each injection with a terminal bleed on day 56. The collected samples were processed for serum by centrifugation and stored at −80° C. until analysis was performed.

xi. HCV Pseudoparticle Generation

HCV pseudoparticles (HCVpp) were generated as described previously (81), by co-transfection of HEK293T cells with the murine leukemia virus (MLV) Gag-Pol packaging vector, luciferase reporter plasmid, and plasmid expressing HCV E1E2 using Lipofectamine 3000 (Thermo Fisher Scientific). Envelope-free control (empty plasmid) was used as negative control in all experiments. Supernatants containing HCVpp were harvested at 48 h and 72 h post-transfection and filtered through 0.45 μm pore-sized membranes. For measurements of serum binding to HCVpp in ELISA, concentrated HCVpp were obtained by ultracentrifugation of 33 ml of filtered supernatants through a 7 ml 20% sucrose cushion using an SW 28 Beckman Coulter rotor at 25,000 rpm for 2.5 hours at 4° C., following a previously reported protocol (42).

xii. HCVpp Neutralization Assays

Huh7 cells were maintained in the Dulbecco's modified Eagle's medium supplemented with 10% FBS. 1.5×104 Huh7 cells per well, plated in white 96-well tissue culture plates (Corning), and incubated overnight at 37° C. The following day, HCVpp was mixed with serial diluted murine serum samples at 37° C. After one-hour incubation, the HCVpp-serum mixture was added to the Huh7 cells (kindly provided by Jonathan K. Ball, University of Nottingham, UK) in 96-well plates and incubated at 37° C. for 5 h. After removing the inoculum, the cells were further incubated for 72 h with DMEM containing 10% fetal bovine serum (Thermo Fisher, Waltham, MA) and the luciferase activities were measured using Bright-Glo™ luciferase assay system as indicated by the manufacturer (Promega, Madison, WI).

xiii. Statistical Comparisons

P-values between group endpoint titers and group ID50 values were calculated in Graphpad Prism software, using non-parametric Kruskal-Wallis analysis of variance with Dunn's multiple comparisons test.

xiv. Computational Design of Coiled Coil Assemblies

Coiled coil assemblies were designed using the HBNet protocol in Rosetta (1). This protocol accepts coiled coil architectures as input, performing modular hydrogen bond network generation and subsequent design to optimize packing and stability, resulting in models of designed assemblies (1). Two architectures were selected for parametric generation of coiled coil bundles for Rosetta input: supercoiled and no supercoil (parallel coil). The supercoil parameters were selected based on the GCN4 leucine zipper structure (PDB code 1ZIK) (2). Backbones were generated with these two architectures using a Python program described previously and available in Rosetta (3), with each helix 30 amino acids in length. By varying helix phases in 18° increments for the inner and outer helices in the Python program, 400 backbones were generated per global architecture (supercoil and parallel coil). As the design subunits in this system were heterodimeric rather than monomeric, we added a minor modification to the published HBNet Rosetta Script protocol (1) to account for the chain break between heterodimeric subunits (“<Span begin=“30” end=“31” bb=“0” chi=“1”/>). HBNet design was performed with each of the 800 input backbone structures, resulting in approximately 335 output designs. Some backbone structures resulted in no output designs due to lack of candidate hydrogen bond networks identified by HBNet, while others resulted in multiple designs based on multiple candidate hydrogen bond networks and packing designs. Design models were assessed for lack of buried unsatisfied polar groups, which has been found to be associated with successful designed assemblies (1), followed by manual inspection, to select the top five candidates for experimental characterization. Sequences for these five designs are given below.

xv. Peptide Synthesis and Characterization

Peptides for coiled coil designs CC1+CC2, HEX-1, HEX-2, HEX-3, and HEX-4 were synthesized (Genscript) and resuspended in Milli-Q water. Pairs of peptides corresponding to each coiled coil design were mixed at a 1:1 ratio and incubated overnight in 4° C. 10×PBS was then added at 1/10th the volume of the mixture, which was centrifuged to separate any precipitate. Each peptide mixture was purified using a Superdex 75 Increase 10/300 column (Cytiva). Elution peak positions of gel filtration standards (Bio-Rad #1511901) using the same column were used to calculate molecular weights of designs CC1+CC2 and HEX-1-4 based on their observed peak positions.

xvi. Sequences

mbE1E2 and sE1E2 amino acid sequences used in the experiments described herein are shown below. mbE1E2, cleavable polyprotein sE1E2 designs, and covalent linker sE1E2 designs are shown in FASTA format, with added or removed portions highlighted. Wild-type E1E2 transmembrane domains (TMDs) in mbE1E2 are shaded gray. Scaffold and linker sequences are underlined, and residues underlined and bolded were added as a short linker between ectodomain and scaffold. Furin cleavage sites (6×Arg) and His tags (6×His) are in lowercase letters.

mbE1E2
(SEQ ID NO: 36)
YQVRNSSGLYHVTNDCPNSSIVYEAADAILHTPGCVPCVREGNASRCWVAVTPTVA
TRDGKLPTTQLRRHIDLLVGSATLCSALYVGDLCGSVFLVGQLFTFSPRRHWTTQDC

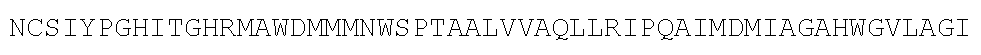

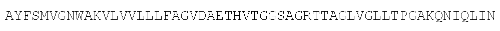

TNGSWHINSTALNCNESLNTGWLAGLFYQHKFNSSGCPERLASCRRLTDFAQGWGPI
SYANGSGLDERPYCWHYPPRPCGIVPAKSVCGPVYCFTPSPVVVGTTDRSGAPTYSW
GANDTDVFVLNNTRPPLGNWFGCTWMNSTGFTKVCGAPPCVIGGVGNNTLLCPTDC
FRKHPEATYSRCGSGPWITPRCMVDYPYRLWHYPCTINYTIFKVRMYVGGVEHRLE
AACNWTRGERCDLEDRDRSELSPLLLSTTQWQVLPCSFTTLPALSTGLIHLHQNIVDV

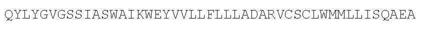

sE1E2.LZ
(SEQ ID NO: 5)
YQVRNSSGLYHVTNDCPNSSIVYEAADAILHTPGCVPCVREGNASRCWVAVTPTVA
TRDGKLPTTQLRRHIDLLVGSATLCSALYVGDLCGSVFLVGQLFTFSPRRHWTTQDC
NCSIYPGHITGHRMAWDMMMNWSPTAALVVAQLLRIPQAIMDMIAPGGRIARLEEK
VKTLKAQNSELASTANMLREQVAQLKQKVMNYrrrrrrETHVTGGSAGRTTAGLVGLL
TPGAKQNIQLININGSWHINSTALNCNESLNTGWLAGLFYQHKFNSSGCPERLASCR
RLTDFAQGWGPISYANGSGLDERPYCWHYPPRPCGIVPAKSVCGPVYCFTPSPVVVG
TTDRSGAPTYSWGANDTDVFVLNNTRPPLGNWFGCTWMNSTGFTKVCGAPPCVIG
GVGNNTLLCPTDCFRKHPEATYSRCGSGPWITPRCMVDYPYRLWHYPCTINYTIFKV
RMYVGGVEHRLEAACNWTRGERCDLEDRDRSELSPLLLSTTQWQVLPCSFTTLPAL
STGLIHLHQNIVDVQYLYGVGSSIASWAIPGGLTDTLQAETDQLEDKKSALQTEIANL
LKEKEKLEFILAAYhhhhhh
sE1E2.FD
(SEQ ID NO: 37)
YQVRNSSGLYHVTNDCPNSSIVYEAADAILHTPGCVPCVREGNASRCWVAVTPTVA
TRDGKLPTTQLRRHIDLLVGSATLCSALYVGDLCGSVFLVGQLFTFSPRRHWTTQDC
NCSIYPGHITGHRMAWDMMMNWSPTAALVVAQLLRIPQAIMDMIAGSGYIPEAPRD
GQAYVRKDGEWVLLSTFLrrrrrrETHVTGGSAGRTTAGLVGLLTPGAKQNIQLINTNG
SWHINSTALNCNESLNTGWLAGLFYQHKFNSSGCPERLASCRRLTDFAQGWGPISYA
NGSGLDERPYCWHYPPRPCGIVPAKSVCGPVYCFTPSPVVVGTTDRSGAPTYSWGA
NDTDVFVLNNTRPPLGNWFGCTWMNSTGFTKVCGAPPCVIGGVGNNTLLCPTDCFR
KHPEATYSRCGSGPWITPRCMVDYPYRLWHYPCTINYTIFKVRMYVGGVEHRLEAA
CNWTRGERCDLEDRDRSELSPLLLSTTQWQVLPCSFTTLPALSTGLIHLHQNIVDVQY
LYGVGSSIASWAIhhhhhh
sE1E2.CC
(SEQ ID NO: 38)
YQVRNSSGLYHVTNDCPNSSIVYEAADAILHTPGCVPCVREGNASRCWVAVTPTVA
TRDGKLPTTQLRRHIDLLVGSATLCSALYVGDLCGSVFLVGQLFTFSPRRHWTTQDC
NCSIYPGHITGHRMAWDMMMNWSPTAALVVAQLLRIPQAIMDMIAAAEDLLELAH
TILKTARNQLRTMEILRKERrrrrrrETHVTGGSAGRTTAGLVGLLTPGAKQNIQLINTNG
SWHINSTALNCNESLNTGWLAGLFYQHKFNSSGCPERLASCRRLTDFAQGWGPISYA
NGSGLDERPYCWHYPPRPCGIVPAKSVCGPVYCFTPSPVVVGTTDRSGAPTYSWGA
NDTDVFVLNNTRPPLGNWFGCTWMNSTGFTKVCGAPPCVIGGVGNNTLLCPTDCFR
KHPEATYSRCGSGPWITPRCMVDYPYRLWHYPCTINYTIFKVRMYVGGVEHRLEAA
CNWTRGERCDLEDRDRSELSPLLLSTTQWQVLPCSFTTLPALSTGLIHLHQNIVDVQY
LYGVGSSIASWAIADERRKAKELLKEAEEIWKRINELAERETKhhhhhh
SE1E2.R6
(SEQ ID NO: 39)
YQVRNSSGLYHVTNDCPNSSIVYEAADAILHTPGCVPCVREGNASRCWVAVTPTVA
TRDGKLPTTQLRRHIDLLVGSATLCSALYVGDLCGSVFLVGQLFTFSPRRHWTTQDC
NCSIYPGHITGHRMAWDMMMNWSPTAALVVAQLLRIPQAIMDMIArrrrrrETHVTGG
SAGRTTAGLVGLLTPGAKQNIQLINTNGSWHINSTALNCNESLNTGWLAGLFYQHKF
NSSGCPERLASCRRLTDFAQGWGPISYANGSGLDERPYCWHYPPRPCGIVPAKSVCG
PVYCFTPSPVVVGTTDRSGAPTYSWGANDTDVFVLNNTRPPLGNWFGCTWMNSTGF
TKVCGAPPCVIGGVGNNTLLCPTDCFRKHPEATYSRCGSGPWITPRCMVDYPYRLW
HYPCTINYTIFKVRMYVGGVEHRLEAACNWTRGERCDLEDRDRSELSPLLLSTTQW
QVLPCSFTTLPALSTGLIHLHQNIVDVQYLYGVGSSIASWAIhhhhhh
sE1E2GS3
(SEQ ID NO: 40)
YQVRNSSGLYHVTNDCPNSSIVYEAADAILHTPGCVPCVREGNASRCWVAVTPTVA
TRDGKLPTTQLRRHIDLLVGSATLCSALYVGDLCGSVFLVGQLFTFSPRRHWTTQDC
NCSIYPGHITGHRMAWDMMMNWSPTAALVVAQLLRIPQAIMDMIAGGGGSGGGGS
GGGGSETHVTGGSAGRTTAGLVGLLTPGAKQNIQLININGSWHINSTALNCNESLNT
GWLAGLFYQHKFNSSGCPERLASCRRLTDFAQGWGPISYANGSGLDERPYCWHYPP
RPCGIVPAKSVCGPVYCFTPSPVVVGTTDRSGAPTYSWGANDTDVFVLNNTRPPLGN
WFGCTWMNSTGFTKVCGAPPCVIGGVGNNTLLCPTDCFRKHPEATYSRCGSGPWIT
PRCMVDYPYRLWHYPCTINYTIFKVRMYVGGVEHRLEAACNWTRGERCDLEDRDR
SELSPLLLSTTQWQVLPCSFTTLPALSTGLIHLHQNIVDVQYLYGVGSSIASWAIhhhhh
h
SE1E2RevGS3
(SEQ ID NO: 41)
ETHVTGGSAGRTTAGLVGLLTPGAKQNIQLININGSWHINSTALNCNESLNTGWLAG
LFYQHKFNSSGCPERLASCRRLTDFAQGWGPISYANGSGLDERPYCWHYPPRPCGIV
PAKSVCGPVYCFTPSPVVVGTTDRSGAPTYSWGANDTDVFVLNNTRPPLGNWFGCT
WMNSTGFTKVCGAPPCVIGGVGNNTLLCPTDCFRKHPEATYSRCGSGPWITPRCMV
DYPYRLWHYPCTINYTIFKVRMYVGGVEHRLEAACNWTRGERCDLEDRDRSELSPL
LLSTTQWQVLPCSFTTLPALSTGLIHLHQNIVDVQYLYGVGSSIASWAIGGGGSGGGG
SGGGGSYQVRNSSGLYHVTNDCPNSSIVYEAADAILHTPGCVPCVREGNASRCWVA
VTPTVATRDGKLPTTQLRRHIDLLVGSATLCSALYVGDLCGSVFLVGQLFTFSPRRH
WTTQDCNCSIYPGHITGHRMAWDMMMNWSPTAALVVAQLLRIPQAIMDMIAhhhhh
h
E1-Jun
(SEQ ID NO: 42)
YQVRNSSGLYHVTNDCPNSSIVYEAADAILHTPGCVPCVREGNASRCWVAVTPTVA
TRDGKLPTTQLRRHIDLLVGSATLCSALYVGDLCGSVFLVGQLFTFSPRRHWTTQDC
NCSIYPGHITGHRMAWDMMMNWSPTAALVVAQLLRIPQAIMDMIAPGGRIARLEEK
VKTLKAQNSELASTANMLREQVAQLKQKVMNY
E2-Fos
(SEQ ID NO: 43)
ETHVTGGSAGRTTAGLVGLLTPGAKQNIQLININGSWHINSTALNCNESLNTGWLAG
LFYQHKFNSSGCPERLASCRRLTDFAQGWGPISYANGSGLDERPYCWHYPPRPCGIV
PAKSVCGPVYCFTPSPVVVGTTDRSGAPTYSWGANDTDVFVLNNTRPPLGNWFGCT
WMNSTGFTKVCGAPPCVIGGVGNNTLLCPTDCFRKHPEATYSRCGSGPWITPRCMV
DYPYRLWHYPCTINYTIFKVRMYVGGVEHRLEAACNWTRGERCDLEDRDRSELSPL
LLSTTQWQVLPCSFTTLPALSTGLIHLHQNIVDVQYLYGVGSSIASWAIPGGLTDTLQ
AETDOLEDKKSALQTEIANLLKEKEKLEFILAAYhhhhhh

The following are amino acid sequences of peptides designed for heterohexameric assembly. Sequences of CC1+CC2, HEX-1, HEX-2, HEX-3, and HEX-4 peptides in FASTA format, with components designed as E1 and E2 scaffolds listed separately.

CC1 (E1)
(SEQ ID NO: 44)
AAEDLLELAHTILKTARNQLRTMEILRKER
CC2 (E2)
(SEQ ID NO: 45)
ADERRKAKELLKEAEEIWKRINELAERETK
HEX-1 (E1)
(SEQ ID NO: 46)
DEEEAVRHNNNVLAKAVEDMLKAVEDNNRH
HEX-1 (E2)
(SEQ ID NO: 47)
DRKEEWDRNAKHIEERAREWLKRMEDRTRE
HEX-2 (E1)
(SEQ ID NO: 48)
DAMKWAMDSNTEVAEMAWRAFHWAVRLREK
HEX-2 (E2)
(SEQ ID NO: 49)
DEEKWFRDSHRRIREWEERMRELYERAERR
HEX-3 (E1)
(SEQ ID NO: 50)
TEKELIKWLAKAMKDAIRIIEENNRWLRES
HEX-3 (E2)
(SEQ ID NO: 51)
DEEAEREWRDLKRWVEELKRRSEEEWRRAN
HEX-4 (E1)
(SEQ ID NO: 52)
SEEEVARHIVKIAEWFRTLVKAFESNVRSQ
HEX-4 (E2)
(SEQ ID NO: 53)
SKKAEDDARKADDEARKAWERLKELLDRQN

Those skilled in the art will recognize, or be able to ascertain using no more than routine experimentation, many equivalents to the specific embodiments of the method and compositions described herein. Such equivalents are intended to be encompassed by the following claims.

### REFERENCES

1. WHO (2017) Global Hepatitis Report 2017. (World Health Organization, Geneva).
2. Waheed Y, Siddiq M, Jamil Z, & Najmi M H (2018) Hepatitis elimination by 2030: Progress and challenges. World J Gastroenterol 24(44):4959-4961.
3. Moosavy S H, et al. (2017) Epidemiology, transmission, diagnosis, and outcome of Hepatitis C virus infection. Electron Physician 9(10):5646-5656.
4. Zaltron S, Spinetti A, Biasi L, Baiguera C, & Castelli F (2012) Chronic HCV infection: epidemiological and clinical relevance. BMC Infect Dis 12 Suppl 2:S2.
5. Ansaldi F, Orsi A, Sticchi L, Bruzzone B, & Icardi G (2014) Hepatitis C virus in the new era: perspectives in epidemiology, prevention, diagnostics and predictors of response to therapy. World J Gastroenterol 20(29):9633-9652.
6. Buhler S & Bartenschlager R (2012) Promotion of hepatocellular carcinoma by hepatitis C virus. Dig Dis 30(5):445-452.
7. Bartenschlager R, et al. (2018) Critical challenges and emerging opportunities in hepatitis C virus research in an era of potent antiviral therapy: Considerations for scientists and funding agencies. Virus Res 248:53-62.
8. Al-Khazraji A, et al. (2020) Identifying Barriers to the Treatment of Chronic Hepatitis C Infection. Dig Dis 38(1):46-52.
9. Roche B, Coilly A, Duclos-Vallee J C, & Samuel D (2018) The impact of treatment of hepatitis C with DAAs on the occurrence of HCC. Liver Int 38 Suppl 1:139-145.
10. Midgard H, et al. (2016) Hepatitis C reinfection after sustained virological response. J Hepatol 64(5):1020-1026.
11. Duncan J D, Urbanowicz R A, Tarr A W, & Ball J K (2020) Hepatitis C Virus Vaccine: Challenges and Prospects. Vaccines (Basel) 8(1).
12. Bailey J R, Barnes E, & Cox A L (2019) Approaches, Progress, and Challenges to Hepatitis C Vaccine Development. Gastroenterology 156(2):418-430.
13. Smith D B, et al. (2014) Expanded classification of hepatitis C virus into 7 genotypes and 67 subtypes: updated criteria and genotype assignment web resource. Hepatology 59(1):318-327.
14. Lavie M, Hanoulle X, & Dubuisson J (2018) Glycan Shielding and Modulation of Hepatitis C Virus Neutralizing Antibodies. Front Immunol 9:910.
15. Helle F, et al. (2010) Role of N-linked glycans in the functions of hepatitis C virus envelope proteins incorporated into infectious virions. J Virol 84(22):11905-11915.
16. Brasher N A, et al. (2020) B cell immunodominance in primary hepatitis C virus infection. Journal of hepatology 72(4):670-679.
17. Cashman S B, Marsden B D, & Dustin L B (2014) The Humoral Immune Response to HCV: Understanding is Key to Vaccine Development. Front Immunol 5:550.
18. Pierce B G, Keck Z Y, & Foung S K (2016) Viral evasion and challenges of hepatitis C virus vaccine development. Current opinion in virology 20:55-63.
19. Prentoe J & Bukh J (2018) Hypervariable Region 1 in Envelope Protein 2 of Hepatitis C Virus: A Linchpin in Neutralizing Antibody Evasion and Viral Entry. Front Immunol 9:2146.
20. Sepulveda-Crespo D, Resino S, & Martinez I (2020) Hepatitis C virus vaccine design: focus on the humoral immune response. J Biomed Sci 27(1):78.
21. Kong L, Jackson K N, Wilson I A, & Law M (2015) Capitalizing on knowledge of hepatitis C virus neutralizing epitopes for rational vaccine design. Current opinion in virology 11:148-157.
22. Penin F, Dubuisson J, Rey F A, Moradpour D, & Pawlotsky J M (2004) Structural biology of hepatitis C virus. Hepatology 39(1):5-19.
23. Lapa D, Garbuglia A R, Capobianchi M R, & Del Porto P (2019) Hepatitis C Virus Genetic Variability, Human Immune Response, and Genome Polymorphisms: Which Is the Interplay? Cells 8(4).
24. Lavie M, Goffard A, & Dubuisson J (2007) Assembly of a functional HCV glycoprotein heterodimer. Curr Issues Mol Biol 9(2):71-86.
25. Falson P, et al. (2015) Hepatitis C Virus Envelope Glycoprotein E1 Forms Trimers at the Surface of the Virion. J Virol 89(20):10333-10346.
26. Cocquerel L, Wychowski C, Minner F, Penin F, & Dubuisson J (2000) Charged residues in the transmembrane domains of hepatitis C virus glycoproteins play a major role in the processing, subcellular localization, and assembly of these envelope proteins. J Virol 74(8):3623-3633.
27. Op De Beeck A, et al. (2000) The transmembrane domains of hepatitis C virus envelope glycoproteins E1 and E2 play a major role in heterodimerization. J Biol Chem 275(40):31428-31437.
28. Bianchi A, Crotta S, Brazzoli M, Foung S K, & Merola M (2011) Hepatitis C virus e2 protein ectodomain is essential for assembly of infectious virions. Int J Hepatol 2011:968161.
29. Haddad J G, et al. (2017) Identification of Novel Functions for Hepatitis C Virus Envelope Glycoprotein E1 in Virus Entry and Assembly. J Virol 91(8).
30. Vieyres G, Dubuisson J, & Pietschmann T (2014) Incorporation of hepatitis C virus E1 and E2 glycoproteins: the keystones on a peculiar virion. Viruses 6(3):1149-1187.
31. Colpitts C C, Tsai P L, & Zeisel M B (2020) Hepatitis C Virus Entry: An Intriguingly Complex and Highly Regulated Process. Int J Mol Sci 21(6).
32. Zeisel M B, Felmlee D J, & Baumert T F (2013) Hepatitis C virus entry. Curr Top Microbiol Immunol 369:87-112.
33. Pileri P, et al. (1998) Binding of hepatitis C virus to CD81. Science 282(5390):938-941.
34. Scarselli E, et al. (2002) The human scavenger receptor class B type I is a novel candidate receptor for the hepatitis C virus. The EMBO journal 21(19):5017-5025.
35. Kinchen V J, et al. (2019) Plasma deconvolution identifies broadly neutralizing antibodies associated with hepatitis C virus clearance. J Clin Invest 130:4786-4796.
36. Tzarum N, Wilson I A, & Law M (2018) The Neutralizing Face of Hepatitis C Virus E2 Envelope Glycoprotein. Front Immunol 9:1315.
37. Wang Y, Keck Z Y, & Foung S K (2011) Neutralizing antibody response to hepatitis C virus. Viruses 3(11):2127-2145.
38. Colbert M D, et al. (2019) Broadly Neutralizing Antibodies Targeting New Sites of Vulnerability in Hepatitis C Virus E1E2. J Virol 93(14).
39. Flyak A I, et al. (2018) HCV Broadly Neutralizing Antibodies Use a CDRH3 Disulfide Motif to Recognize an E2 Glycoprotein Site that Can Be Targeted for Vaccine Design. Cell Host Microbe 24(5):703-716 e703.
40. Keck Z Y, et al. (2019) Broadly neutralizing antibodies from an individual that naturally cleared multiple hepatitis C virus infections uncover molecular determinants for E2 targeting and vaccine design. PLoS Pathog 15(5):e1007772.
41. Law M, et al. (2008) Broadly neutralizing antibodies protect against hepatitis C virus quasispecies challenge. Nat Med 14(1):25-27.
42. Keck Z Y, et al. (2005) Analysis of a highly flexible conformational immunogenic domain a in hepatitis C virus E2. J Virol 79(21):13199-13208.
43. Li Y, et al. (2015) Structural basis for penetration of the glycan shield of hepatitis C virus E2 glycoprotein by a broadly neutralizing human antibody. J Biol Chem 290(16):10117-10125.
44. Vasiliauskaite I, et al. (2017) Conformational Flexibility in the Immunoglobulin-Like Domain of the Hepatitis C Virus Glycoprotein E2. MBio 8(3).
45. Stroh L J, Nagarathinam K, & Krey T (2018) Conformational Flexibility in the CD81-Binding Site of the Hepatitis C Virus Glycoprotein E2. Front Immunol 9:1396.
46. Giang E, et al. (2012) Human broadly neutralizing antibodies to the envelope glycoprotein complex of hepatitis C virus. Proc Natl Acad Sci USA 109(16):6205-6210.
47. Chen F, et al. (2020) Antibody Responses to Immunization With HCV Envelope Glycoproteins as a Baseline for B-Cell-Based Vaccine Development. Gastroenterology 158(4):1058-1071 e1056.
48. Fauvelle C, et al. (2016) Hepatitis C virus vaccine candidates inducing protective neutralizing antibodies. Expert Rev Vaccines 15(12):1535-1544.
49. Fuerst T R, Pierce B G, Keck Z Y, & Foung S K H (2017) Designing a B Cell-Based Vaccine against a Highly Variable Hepatitis C Virus. Frontiers in microbiology 8:2692.
50. Zazrin H, Shaked H, & Chill J H (2014) Architecture of the hepatitis C virus E1 glycoprotein transmembrane domain studied by NMR. Biochim Biophys Acta 1838(3):784-792.
51. Kong L, et al. (2013) Hepatitis C virus E2 envelope glycoprotein core structure. Science 342(6162):1090-1094.
52. Kong L, et al. (2015) Structure of Hepatitis C Virus Envelope Glycoprotein E1 Antigenic Site 314-324 in Complex with Antibody IGH526. J Mol Biol 427(16):2617-2628.
53. Spadaccini R, et al. (2010) Structural characterization of the transmembrane proximal region of the hepatitis C virus E1 glycoprotein. Biochimica et biophysica acta 1798(3):344-353.
54. Tzarum N, et al. (2019) Genetic and structural insights into broad neutralization of hepatitis C virus by human VH1-69 antibodies. Sci Adv 5(1):eaav1882.
55. McLellan J S, et al. (2013) Structure-based design of a fusion glycoprotein vaccine for respiratory syncytial virus. Science 342(6158):592-598.
56. Sanders R W, et al. (2013) A next-generation cleaved, soluble HIV-1 Env trimer, BG505 SOSIP.664 gp140, expresses multiple epitopes for broadly neutralizing but not non-neutralizing antibodies. PLoS Pathog 9(9):e1003618.
57. Yassine H M, et al. (2015) Hemagglutinin-stem nanoparticles generate heterosubtypic influenza protection. Nat Med 21(9):1065-1070.
58. Rutten L, et al. (2020) Structure-Based Design of Prefusion-Stabilized Filovirus Glycoprotein Trimers. Cell Rep 30(13):4540-4550 e4543.
59. Slon-Campos J L, et al. (2019) A protective Zika virus E-dimer-based subunit vaccine engineered to abrogate antibody-dependent enhancement of dengue infection. Nat Immunol 20(10):1291-1298.
60. Graham B S, Gilman M S A, & McLellan J S (2019) Structure-Based Vaccine Antigen Design. Annu Rev Med 70:91-104.
61. Kanekiyo M & Graham B S (2020) Next-Generation Influenza Vaccines. Cold Spring Harb Perspect Med.
62. Jones L D, Moody M A, & Thompson A B (2020) Innovations in HIV-1 Vaccine Design. Clin Ther.
63. Lu Y, Welsh J P, & Swartz J R (2014) Production and stabilization of the trimeric influenza hemagglutinin stem domain for potentially broadly protective influenza vaccines. Proc Natl Acad Sci USA 111(1):125-130.
64. Kim E, et al. (2020) Microneedle array delivered recombinant coronavirus vaccines: Immunogenicity and rapid translational development. EBioMedicine: 102743.
65. Tai W, et al. (2016) A recombinant receptor-binding domain of MERS-CoV in trimeric form protects human dipeptidyl peptidase 4 (hDPP4) transgenic mice from MERS-CoV infection. Virology 499:375-382.
66. Chang Y C, et al. (2018) Efficacy of heat-labile enterotoxin B subunit-adjuvanted parenteral porcine epidemic diarrhea virus trimeric spike subunit vaccine in piglets. Appl Microbiol Biotechnol 102(17):7499-7507.
67. Leblanc P, et al. (2014) VaxCelerate II: rapid development of a self-assembling vaccine for Lassa fever. Hum Vaccin Immunother 10(10):3022-3038.
68. Ruwona T B, Giang E, Nieusma T, & Law M (2014) Fine mapping of murine antibody responses to immunization with a novel soluble form of hepatitis C virus envelope glycoprotein complex. J Virol 88(18):10459-10471.
69. Cai W, et al. (2010) Expression, purification and immunogenic characterization of hepatitis C virus recombinant E1E2 protein expressed by Pichia pastoris yeast. Antiviral research 88(1):80-85.
70. Logan M, et al. (2017) Native Folding of a Recombinant gpE1/gpE2 Heterodimer Vaccine Antigen from a Precursor Protein Fused with Fc IgG. J Virol 91(1).
71. Cao L, et al. (2019) Functional expression and characterization of the envelope glycoprotein E1E2 heterodimer of hepatitis C virus. PLoS Pathog 15(5):e1007759.
72. Wen B, et al. (2011) Signal peptide replacements enhance expression and secretion of hepatitis C virus envelope glycoproteins. Acta Biochim Biophys Sin (Shanghai) 43(2):96-102.
73. Glover J N & Harrison S C (1995) Crystal structure of the heterodimeric bZIP transcription factor c-Fos-c-Jun bound to DNA. Nature 373(6511):257-261.
74. Willcox B E, et al. (1999) Production of soluble alphabeta T-cell receptor heterodimers suitable for biophysical analysis of ligand binding. Protein Sci 8(11):2418-2423.
75. Berthelmann A, Lach J, Grawert M A, Groll M, & Eichler J (2014) Versatile C(3)-symmetric scaffolds and their use for covalent stabilization of the foldon trimer. Org Biomol Chem 12(16):2606-2614.
76. Wrapp D, et al. (2020) Cryo-EM structure of the 2019-nCoV spike in the prefusion conformation. Science 367(6483):1260-1263.
77. Boyken S E, et al. (2016) De novo design of protein homo-oligomers with modular hydrogen-bond network-mediated specificity. Science 352(6286):680-687.
78. Keck Z Y, et al. (2004) Human monoclonal antibody to hepatitis C virus E1 glycoprotein that blocks virus attachment and viral infectivity. J Virol 78(13):7257-7263.
79. Broering T J, et al. (2009) Identification and characterization of broadly neutralizing human monoclonal antibodies directed against the E2 envelope glycoprotein of hepatitis C virus. J Virol 83(23):12473-12482.
80. Iacob R E, Perdivara I, Przybylski M, & Tomer K B (2008) Mass spectrometric characterization of glycosylation of hepatitis C virus E2 envelope glycoprotein reveals extended microheterogeneity of N-glycans. J Am Soc Mass Spectrom 19(3):428-444.
81. Urbanowicz R A, et al. (2019) Antigenicity and Immunogenicity of Differentially Glycosylated Hepatitis C Virus E2 Envelope Proteins Expressed in Mammalian and Insect Cells. J Virol 93(7).
82. Gandhi A V, Pothecary M R, Bain D L, & Carpenter J F (2017) Some Lessons Learned From a Comparison Between Sedimentation Velocity Analytical Ultracentrifugation and Size Exclusion Chromatography to Characterize and Quantify Protein Aggregates. J Pharm Sci 106(8):2178-2186.
83. Freedman H, et al. (2017) Computational Prediction of the Heterodimeric and Higher-Order Structure of gpE1/gpE2 Envelope Glycoproteins Encoded by Hepatitis C Virus. J Virol 91(8).
84. Keck Z Y, et al. (2012) Human monoclonal antibodies to a novel cluster of conformational epitopes on HCV E2 with resistance to neutralization escape in a genotype 2a isolate. PLoS Pathog 8(4):e1002653.
85. Keck Z Y, et al. (2011) Mapping a region of hepatitis C virus E2 that is responsible for escape from neutralizing antibodies and a core CD81-binding region that does not tolerate neutralization escape mutations. J Virol 85(20):10451-10463.
86. Gopal R, et al. (2017) Probing the antigenicity of hepatitis C virus envelope glycoprotein complex by high-throughput mutagenesis. PLoS Pathog 13(12):e1006735.
87. Khan A G, et al. (2014) Structure of the core ectodomain of the hepatitis C virus envelope glycoprotein 2. Nature 509(7500):381-384.
88. Wahid A, et al. (2013) Disulfide bonds in hepatitis C virus glycoprotein E1 control the assembly and entry functions of E2 glycoprotein. J Virol 87(3):1605-1617.
89. Dubuisson J & Rice C M (1996) Hepatitis C virus glycoprotein folding: disulfide bond formation and association with calnexin. J Virol 70(2):778-786.
90. Angel P & Karin M (1991) The role of Jun, Fos and the AP-1 complex in cell-proliferation and transformation. Biochim Biophys Acta 1072(2-3):129-157.
91. Karin M, Liu Z, & Zandi E (1997) AP-1 function and regulation. Curr Opin Cell Biol 9(2):240-246.
92. Swanson K A, et al. (2020) A respiratory syncytial virus (RSV) F protein nanoparticle vaccine focuses antibody responses to a conserved neutralization domain. Sci Immunol 5(47).
93. Marcandalli J, et al. (2019) Induction of Potent Neutralizing Antibody Responses by a Designed Protein Nanoparticle Vaccine for Respiratory Syncytial Virus. Cell 176(6):1420-1431 e1417.
94. Owsianka A M, et al. (2006) Identification of conserved residues in the E2 envelope glycoprotein of the hepatitis C virus that are critical for CD81 binding. J Virol 80(17):8695-8704.
95. McCaffrey K, et al. (2017) An Optimized Hepatitis C Virus E2 Glycoprotein Core Adopts a Functional Homodimer That Efficiently Blocks Virus Entry. J Virol 91(5).
96. Marin M Q, et al. (2020) Optimized Hepatitis C Virus (HCV) E2 Glycoproteins and their Immunogenicity in Combination with MVA-HCV. Vaccines (Basel) 8(3).
97. Augestad E H, et al. (2020) Global and local envelope protein dynamics of hepatitis C virus determine broad antibody sensitivity. Sci Adv 6(35):eabb5938.
98. Pierce B G, et al. (2020) Structure-based design of hepatitis C virus E2 glycoprotein improves serum binding and cross-neutralization. J Virol.
99. Keck Z, et al. (2013) Cooperativity in virus neutralization by human monoclonal antibodies to two adjacent regions located at the amino terminus of hepatitis C virus E2 glycoprotein. J Virol 87(1):37-51.
100. Keck Z Y, et al. (2004) Hepatitis C virus E2 has three immunogenic domains containing conformational epitopes with distinct properties and biological functions. J Virol 78(17):9224-9232.
101. Keck Z Y, et al. (2016) Affinity maturation of a broadly neutralizing human monoclonal antibody that prevents acute hepatitis C virus infection in mice. Hepatology 64(6):1922-1933.
102. Lebowitz J, Lewis M S, & Schuck P (2002) Modern analytical ultracentrifugation in protein science: a tutorial review. Protein Sci 11(9):2067-2079.
103. Laue™, Shah B D, Ridgeway™, & Pelletier S L (1992) Analytical ultracentrifhgation in biochemistry and polymer science (Royal Society of Chemistry).
104. Andrianov A K, et al. (2020) Supramolecular assembly of Toll-like receptor 7/8 agonist into multimeric water-soluble constructs enables superior immune stimulation in vitro and in vivo. ACS Appl. Bio Mater. 3(5):3187-3195.
105. Andrianov A K, Svirkin Y Y, & LeGolvan M P (2004) Synthesis and biologically relevant properties of polyphosphazene polyacids. Biomacromolecules 5(5):1999-2006.
106. Andrianov A K, Marin A, & Fuerst T R (2016) Molecular-Level Interactions of Polyphosphazene Immunoadjuvants and Their Potential Role in Antigen Presentation and Cell Stimulation. Biomacromolecules 17(11):3732-3742.
107. Wang G, de Jong R N, van den Bremer E T J, Parren P, & Heck A J R (2017) Enhancing Accuracy in Molecular Weight Determination of Highly Heterogeneously Glycosylated Proteins by Native Tandem Mass Spectrometry. Anal Chem 89(9):4793-4797.
108. Pierce B G, et al. (2016) Global mapping of antibody recognition of the hepatitis C virus E2 glycoprotein: Implications for vaccine design. Proc Natl Acad Sci USA 113(45):E6946-E6954.
109. Wang Y, et al. (2011) Affinity maturation to improve human monoclonal antibody neutralization potency and breadth against hepatitis C virus. J Biol Chem 286(51):44218-44233.

1. A modified hepatitis C virus (HCV) E1E2 glycoprotein comprising:
a. a HCV E1 polypeptide, wherein the HCV E1 polypeptide does not comprise a transmembrane domain,
b. a first scaffold element,
c. a HCV E2 polypeptide, wherein the HCV E2 polypeptide does not comprise a transmembrane domain, and
d. a second scaffold element.

2. The modified HCV E1E2 glycoprotein of claim 1, wherein the first and second scaffold elements are capable of interacting with each other.

3. The modified HCV E1E2 glycoprotein of claim 1, wherein the first scaffold element is located on the C-terminus of the HCV E1 polypeptide.

4. The modified HCV E1E2 glycoprotein of claim 1, wherein the second scaffold element is located on the C-terminus of the HCV E2 polypeptide.

5. The modified HCV E1E2 glycoprotein of claim 1, further comprising a cleavage site.

6. The modified HCV E1E2 glycoprotein of claim 5, wherein the cleavage site is located between the HCV E1 polypeptide and the HCV E2 polypeptide.

7. The modified HCV E1E2 glycoprotein of claim 1, wherein the first scaffold element and second scaffold element are not transmembrane domains.

8. The modified HCV E1E2 glycoprotein of, further comprising a leader sequence at the N-terminal end of the HCV E1 polypeptide.

9. The modified HCV E1E2 glycoprotein of claim 8, wherein the leader sequence is a tissue plasminogen activator (tPA) leader sequence.

10. (canceled)

11. (canceled)

12. The modified HCV E1E2 glycoprotein of claim 1, wherein the first scaffold and second scaffold are capable of forming a leucine zipper.

13. (canceled)

14. The modified HCV E1E2 glycoprotein of claim 1, wherein the first scaffold is a first coiled-coil domain and the second scaffold is a second coiled-coil domain.

15. (canceled)

16. (canceled)

17. (canceled)

18. (canceled)

19. The modified HCV E1E2 glycoprotein of claim 1, wherein the HCV E1 polyprotein comprises the sequence of SEQ ID NO:1 and/or wherein the HCV E2 polyprotein comprises the sequence of SEQ ID NO:2.

20. (canceled)

21. The modified HCV E1E2 glycoprotein of claim 1, wherein the modified HCV E1E2 glycoprotein comprises the sequence of SEQ ID NO:5.

22. A modified hepatitis C virus (HCV) E1E2 glycoprotein comprising:
a. a HCV E1 polypeptide, wherein the HCV E1 polypeptide does not comprise a transmembrane domain,
b. a first scaffold element,
c. a modified HCV E2 polypeptide, wherein the modified HCV E2 polypeptide does not comprise a transmembrane domain,
wherein the modified HCV E2 polypeptide comprises an antigenic domain D,
wherein the modified HCV E2 polypeptide comprises one or more amino acid alterations in the antigenic domain D, and
d. a second scaffold element.

23.-34. (canceled)

35. A modified hepatitis C virus (HCV) E1E2 glycoprotein comprising:
a. a HCV E1 polypeptide, wherein the HCV E1 polypeptide does not comprise a transmembrane domain,
b. a first scaffold,
c. a HCV E2 polypeptide, wherein the HCV E2 polypeptide does not comprise a transmembrane domain,
wherein the HCV E2 polypeptide comprises an antigenic domain A, wherein the antigenic domain A comprises an N-glycan sequon substitution, and
d. a second scaffold.

36.-43. (canceled)

44. A method of increasing HCV E1E2 glycoprotein immunogenicity in a subject in need thereof comprising administering to the subject in need thereof a composition comprising one or more of the modified HCV E1E2 glycoproteins of claim 1.

45. A method of inducing neutralizing antibodies (nAbs) in a subject in need thereof comprising administering to the subject in need thereof a composition comprising one or more of the modified HCV E1E2 glycoproteins of claim 1 or the composition of claim 43.

46.-50. (canceled)

51. A method of inducing an immune response in a subject in need thereof comprising administering to the subject in need thereof a composition comprising one or more of the modified HCV E1E2 glycoproteins of claim 1.

52. (canceled)

53. A method of treating a subject having HCV comprising administering to the subject a composition comprising one or more of the modified HCV E1E2 glycoproteins of claim 1.

54. (canceled)

55. A method for immunizing a subject comprising: administering to the subject a composition comprising one or more of the modified HCV E1E2 glycoproteins of claim 1.

56. (canceled)

57. (canceled)

58. (canceled)


SEQUENCE LISTING (59 sequences)

SEQ ID NO: 1 - protein, 158 aa, Artificial Sequence
  synthetic construct; HCV E1 polypeptide
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIA

SEQ ID NO: 2 - protein, 331 aa, Artificial Sequence
  synthetic construct; HCV E2 polypeptide
ETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAI

SEQ ID NO: 3 - protein, 30 aa, Artificial Sequence
  synthetic construct; coiled-coil domain
AAEDLLELAHTILKTARNQLRTMEILRKER

SEQ ID NO: 4 - protein, 30 aa, Artificial Sequence
  synthetic construct; coiled-coil domain
ADERRKAKELLKEAEEIWKRINELAERETK

SEQ ID NO: 5 - protein, 587 aa, Artificial Sequence
  synthetic construct; modified HCV E1E2 glycoproteins
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAPGGRIARLEEKVKTLKAQNSELASTANMLREQVAQLKQKVMNYRRRRRRETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIPGGLTDTLQAETDQLEDKKSALQTEIANLLKEKEKLEFILAAYHHHHHH

SEQ ID NO: 6 - protein, 331 aa, Artificial Sequence
  synthetic construct; modified HCV E1E2 glycoproteins
ETHVTGGSAGRTTAGLVGLLTPGAKQNIQLINTNGSWHINSTALNCNESLNTGWLAGLFYQPKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAI

SEQ ID NO: 7 - protein, 331 aa, Artificial Sequence
  synthetic construct; modified HCV E2 polypeptide
ETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMNVSGVEHRLEAACNWTRGERCDLEDRDRSELSPLLLSTTQWQVLPCSFTTLPALSTGLIHLHQNIVDVQYLYGVGSSIASWAI

SEQ ID NO: 8 - protein, 40 aa, Artificial Sequence
  synthetic construct; c-Jun sequence
RIARLEEKVKTLKAQNSELASTANMLREQVAQLKQKVMNY

SEQ ID NO: 9 - protein, 40 aa, Artificial Sequence
  synthetic construct; c-Fos sequence
LTDTLQAETDQLEDKKSALQTEIANLLKEKEKLEFILAAY

SEQ ID NO: 10 - protein, 43 aa, Artificial Sequence
  synthetic construct; c-Jun sequence
PGGRIARLEEKVKTLKAQNSELASTANMLREQVAQLKQKVMNY

SEQ ID NO: 11 - protein, 43 aa, Artificial Sequence
  synthetic construct; c-Fos sequence
PGGLTDTLQAETDQLEDKKSALQTEIANLLKEKEKLEFILAAY

SEQ ID NO: 12 - protein, 6 aa, Artificial Sequence
  synthetic construct; cleavage site
RRRRRR

SEQ ID NO: 13 - protein, 6 aa, Artificial Sequence
  synthetic construct; cleavage site
RRRRKR

SEQ ID NO: 14 - protein, 6 aa, Artificial Sequence
  synthetic construct; cleavage site
RRRKKR

SEQ ID NO: 15 - protein, 4 aa, Artificial Sequence
  synthetic construct; cleavage site
  misc_feature (2)..(2): Xaa can be any naturally occurring amino acid
RXKR

SEQ ID NO: 16 - protein, 4 aa, Artificial Sequence
  synthetic construct; cleavage site
  misc_feature (2)..(2): Xaa can be any naturally occurring amino acid
RXRR

SEQ ID NO: 17 - protein, 7 aa, Artificial Sequence
  synthetic construct; cleavage site
ENLYFQS

SEQ ID NO: 18 - protein, 8 aa, Artificial Sequence
  synthetic construct; cleavage site
LEVLFQGP

SEQ ID NO: 19 - protein, 25 aa, Artificial Sequence
  synthetic construct; leader sequence
MGVLLTQRTLLSLVLALLFPSMASM

SEQ ID NO: 20 - protein, 16 aa, Artificial Sequence
  synthetic construct; leader sequence
MKCLLYLAFLFIGVNC

SEQ ID NO: 21 - protein, 21 aa, Artificial Sequence
  synthetic construct; leader sequence
METDTLLLWVLLLWVPGSTGD

SEQ ID NO: 22 - protein, 19 aa, Artificial Sequence
  synthetic construct; leader sequence
MGWSCIILFLVATATGVHS

SEQ ID NO: 23 - protein, 17 aa, Artificial Sequence
  synthetic construct; leader sequence
MRAWIFFLLCLAGRALA

SEQ ID NO: 24 - protein, 21 aa, Artificial Sequence
  synthetic construct; leader sequence
MWWRLWWLLLLLLLLWPMVWA

SEQ ID NO: 25 - protein, 22 aa, Artificial Sequence
  synthetic construct; leader sequence
MDMRVPAQLLGLLLLWLRGARC

SEQ ID NO: 26 - protein, 16 aa, Artificial Sequence
  synthetic construct; leader sequence
MPLLLLLPLLWAGALA

SEQ ID NO: 27 - protein, 23 aa, Artificial Sequence
  synthetic construct; leader sequence
MDAMKRGLCCVLLLCGAVFVSPS

SEQ ID NO: 28 - protein, 18 aa, Artificial Sequence
  synthetic construct; leader sequence
MAFLWLLSCWALLGTTFG

SEQ ID NO: 29 - protein, 15 aa, Artificial Sequence
  synthetic construct; leader sequence
MNLLLILTFVAAAVA

SEQ ID NO: 30 - protein, 20 aa, Artificial Sequence
  synthetic construct; leader sequence
MYRMQLLSCIALSLALVTNS

SEQ ID NO: 31 - protein, 17 aa, Artificial Sequence
  synthetic construct; leader sequence
MGVKVLFALICIAVAEA

SEQ ID NO: 32 - protein, 16 aa, Artificial Sequence
  synthetic construct; leader sequence
MKWVTFISLLFSSAYS

SEQ ID NO: 33 - protein, 16 aa, Artificial Sequence
  synthetic construct; leader sequence
MKTIIALSYIFCLVLG

SEQ ID NO: 34 - protein, 24 aa, Artificial Sequence
  synthetic construct; leader sequence
MALWMRLLPLLALLALWGPDPAAA

SEQ ID NO: 35 - protein, 16 aa, Artificial Sequence
  synthetic construct; leader sequence
MKPIFLVLLVVTSAYA

SEQ ID NO: 36 - protein, 555 aa, Artificial Sequence
  synthetic construct; membrane bound E1/E2
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 37 - protein, 530 aa, Artificial Sequence
  synthetic construct; modified E1/E2
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGSGYIPEAPRDGQAYVRKDGEWVLLSTFLRRRRRRETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIHHHHHH

SEQ ID NO: 38 - protein, 561 aa, Artificial Sequence
  synthetic construct; modified E/E2
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAAAEDLLELAHTILKTARNQLRTMEILRKERRRRRRRETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIADERRKAKELLKEAEEIWKRINELAERETKHHHHHH

SEQ ID NO: 39 - protein, 501 aa, Artificial Sequence
  synthetic construct; modified E1/E2
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIARRRRRRETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIHHHHHH

SEQ ID NO: 40 - protein, 510 aa, Artificial Sequence
  synthetic construct; modified E1/E2
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGGGGSGGGGSGGGGSETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIHHHHHH

SEQ ID NO: 41 - protein, 510 aa, Artificial Sequence
  synthetic construct; modified E1/E2
ETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIGGGGSGGGGSGGGGSYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAHHHHHH

SEQ ID NO: 42 - protein, 201 aa, Artificial Sequence
  synthetic construct; E1-Jun
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAPGGRIARLEEKVKTLKAQNSELASTANMLREQVAQLKQKVMNY

SEQ ID NO: 43 - protein, 380 aa, Artificial Sequence
  synthetic construct; E2-fos
ETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIPGGLTDTLQAETDQLEDKKSALQTEIANLLKEKEKLEFILAAYHHHHHH

SEQ ID NO: 44 - protein, 30 aa, Artificial Sequence; identical to SEQ ID NO: 3
  synthetic construct; coiled coil domain

SEQ ID NO: 45 - protein, 30 aa, Artificial Sequence; identical to SEQ ID NO: 4
  synthetic construct; coiled coil domain

SEQ ID NO: 46 - protein, 30 aa, Artificial Sequence
  synthetic construct; Hex-1
DEEEAVRHNNNVLAKAVEDMLKAVEDNNRH

SEQ ID NO: 47 - protein, 30 aa, Artificial Sequence
  synthetic construct; hex-1
DRKEEWDRNAKHIEERAREWLKRMEDRTRE

SEQ ID NO: 48 - protein, 30 aa, Artificial Sequence
  synthetic construct; hex-2
DAMKWAMDSNTEVAEMAWRAFHWAVRLREK

SEQ ID NO: 49 - protein, 30 aa, Artificial Sequence
  synthetic construct; Hex-2
DEEKWFRDSHRRIREWEERMRELYERAERR

SEQ ID NO: 50 - protein, 30 aa, Artificial Sequence
  synthetic construct; hex-3
TEKELIKWLAKAMKDAIRIIEENNRWLRES

SEQ ID NO: 51 - protein, 30 aa, Artificial Sequence
  synthetic construct; hex-3
DEEAEREWRDLKRWVEELKRRSEEEWRRAN

SEQ ID NO: 52 - protein, 30 aa, Artificial Sequence
  synthetic construct; hex-4
SEEEVARHIVKIAEWFRTLVKAFESNVRSQ

SEQ ID NO: 53 - protein, 30 aa, Artificial Sequence
  synthetic construct; hex-4
SKKAEDDARKADDEARKAWERLKELLDRQN

SEQ ID NO: 54 - protein, 13 aa, Artificial Sequence
  synthetic construct; E2 domain D
NTGWLAGLFYQHK

SEQ ID NO: 55 - protein, 15 aa, Artificial Sequence
  synthetic construct; E2 domain E
NIQLINTNGSWHINS

SEQ ID NO: 56 - protein, 40 aa, Artificial Sequence
  synthetic construct; E2 domain E
ETHVTGGSAGRTTAGLVGLLTPGAKQNIQLINTNGSWHIN

SEQ ID NO: 57 - protein, 15 aa, Artificial Sequence
  synthetic construct; E1 N-terminus
YQVRNSSGLYHVTND

SEQ ID NO: 58 - protein, 12 aa, Artificial Sequence
  synthetic construct; E1 ectodomain
TGHRMAWDMMMN

SEQ ID NO: 59 - protein, 6 aa, Artificial Sequence
  synthetic construct; His tag
HHHHHH